(19) United States
(12) Patent Application Publication
(10) Pub. No.: US 2003/0199070 A1
(43) Pub. Date: Oct. 23, 2003

(54) Crystallization of ribosome recycling factor (RRF) and method of developing inhibitor of RRF

(76) Inventors: Se Won Suh, Seoul (KR); Kyeong Kyu Kim, Seoul (KR); Kyeong Sik Min, Chinju (KR); Sung Chul Ha, Chinju (KR); Soo Hyun Eom, Seoul (KR); Wook Hyun Kim, Seoul (KR); Jung Min Yun, Seoul (KR)

Correspondence Address: KNOBBE MARTENS OLSON & BEAR LLP, 2040 MAIN STREET, FOURTEENTH FLOOR, IRVINE, CA, 92614, US

(21) Appl. No.: 10/357,884

(22) Filed: Feb. 3, 2003

Related U.S. Application Data
(63) Continuation of application No. 09/731,487, filed on Dec. 7, 2000, now abandoned.

(30) Foreign Application Priority Data
Jul. 1, 2000 (KR) 2000-37527

(51) Int. Cl. G06G 7/48; G06G 7/58; G06F 19/00; G01N 33/48; G01N 33/50; C12N 9/22

(52) U.S. Cl. 435/199; 702/20; 703/11

(57) ABSTRACT

There are disclosed a crystal of complex of RRF with a detergent, its crystallization method and a method for identifying an inhibitor for RRF by using the three-dimensional structure of RRF analyzed from the crystal. In the present invention, a crystal of RRF complexed with a detergent is obtained in the presence of the detergent, and the three-dimensional structure of RRF is analyzed from the crystal. There are identified an active site forming hydrophobic cleft and binding site of RRF to A-site of ribosome in the three-dimensional structure of RRF. The three-dimensional structure of RRF, specifically, the active site and binding site can allow an inhibitor for RRF to be designed and identified efficiently. RRF is essential for viability in prokaryotes, but is not essential in eukaryotes. From this fact, the inhibitor for RRF can be used as an antibiotic.

### RELATED APPLICATION

This application is a continuation of application Ser. No. 09/731,487, filed Dec. 7, 2000, now pending, which is hereby incorporated herein in its entirety.

### BACKGROUND OF THE INVENTION

1. Field of the Invention

The present invention relates to a crystal of ribosome recycling factor (RRF) and its crystallization method; and, more particularly, to a crystal of RRF formed by binding with a detergent and its crystallization method. Further, the present invention relates to a protein complex comprised of RRF or RRF homolog and a molecule or material binding to hydrophobic cleft formed between the two domains of RRF. The present invention also relates to polypeptide comprising the structure of one domain of RRF binding to ribosome. The present invention also relates to a method for screening an RRF inhibitor by using three-dimensional structure of RRF. The present invention also relates to a RRF inhibitor including alkyl-β-D-maltopyranoside or its analogs.

2. Description of the Related Technology

After penicillin had been used as an antibiotic with modern concept, several antibiotic materials have been developed to control pathogens. However, appearance of new pathogens and antibiotic-resistant pathogens has continuously brought up the needs for developing new antibiotics. The antibiotics for the purpose of suppressing the metabolism of bacteria such as the synthesis of cell wall or protein biosynthesis has been mainly developed from natural compounds or their derivatives.

Recently, advances in protein crystallography and computation chemistry have introduced the new method of structure based drug design into the field of drug development. The new method is to find inhibitors by fitting inhibitor candidates to the active site of target protein based on the high resolution crystal structure and computing the binding intensities or affinities in the complexes with several manners. This approach is capable of being applied into the researches for developing new antibiotics.

As well known, protein biosynthesis consists of three steps: initiation, elongation and termination. In the termination step, releasing factor (RF1 and RF2 in prokaryote and eRF1 in eukaryote) helps to release the synthesized protein after staying at stop codon, and then is released from A-site of ribosome by releasing factor 3 (RF3) (Freistroffer et al., 1997). Then, other factors remaining in the termination complex must be recycled so that the protein biosynthesis can proceed continuously (Kaji et al. 1998). Ribosome recycling factor (RRF) plays a necessary role in catalyzing the hydrolysis of GTP and the release of mRNA and tRNA from ribosome with help of elongation factor G (EF-G) or release factor 3 (RF3) in the termination step of protein biosynthesis (Pavlov et al., 1997a; Kaji et al., 1998; Grentzmann et al., 1998; FIG. 1). It has been found that RRF directly dissociates peptidyl-tRNA as well as deacylated tRNA from ribosome (Heurgue-Hamard et al., 1998; Grentzmann et al., 1998). It has also been known that RRF plays a role in preventing errors generated in elongation step of protein synthesis (Janosi et al., 1996). The importance of recycling ribosome in the protein synthesis is evidenced by the fact that growth of bacteria is suppressed when the activity of RRF is inhibited (Janosi et al., 1998). The absence of RRF caused cell death in Escherichia coli (Janosi et al., 1994), which might be due to increased translation errors (Janosi et al., 1996) and unscheduled reinitiation (Ryoji et al., 1981; Janosi et al., 1998).

There has not known the mechanism for the prevention of errors in the translation process or recycling of ribosome. It has been found that RRF binds to A-site of ribosome and such binding is competitive with the binding of releasing factor 1 (RF1) (Pavlov et al., 1997b). So, it is predicted based on the binding force to ribosome that RRF will have structural similarity to other translation factors, which bind to A-site of ribosome. Therefore, it is considered that RRF also has the tRNA-mimicry structure proposed in other translation factors (Nakamurat et al., 1996; Brock et al., 1998). According to a model for recycling of ribosome proposed presently, RRF is moved to P-site after binding to A-site of ribosome (Nakamura et al., 1996), and deacylated tRNA is moved to E-site (Janosi et al., 1996; Pavolv et al., 1997a). After this, the complex is dissociated into mRNA, tRNA and ribosome (Kaji et al., 1998), or dissociated into two units of tRNA and ribosome binding with mRNA (Pavolv et al., 1997a). A mechanism proposed by a more recent research is different from this (Karimi et al., 1999), in which RRF, EF-G and GTP only catalyze the dissociation of the 50S subunit from the termination complex, and tRNA is removed from 30S:mRNA:tRNA complex by an initiation factor, IF3.

Genes encoding RRFs are widely distributed in prokaryotes with very high homologies (Janosi et al., 1996). As a result of sequence analysis, it has been discovered that the genes encoding RRFs also exist in eukaryotes. However, those are mostly present in organelles (Janosi et al., 1996). It has been known that RRF homolog in yeast participates in protein synthesis in mitochondria (Janai et al., 1998), but is not essential for cell growth (Kaji et al., 1998). An RRF homolog located in the chloroplast of spinach exerts an inhibitory action on the bacterial temperature-sensitive mutant (Rolland et al., 1999). It may have been concluded from such results that eukariotic RRF is not concerned with cell viability and the inhibition of the RRFs located in organelles does not influence the synthesis of cytoplasmic proteins. This fact is evidenced by the fact that RRF does not exist in Methanococcus jannashchii, which is an ancient creature having the translation system similar to eukaryote. Therefore, it is considered that RRF is a target for developing new antibacterial drugs (Kaji et al., 1998), since it is essential for the cell viability in prokaryotes, but not essential in eukaryotes. Though such an inhibitor may influence the mitochondria of eukaryotes, the inhibitor for RRF can be utilized substantially in view of the fact that widely used antibiotics such as tetracycline and erythromycin also target the protein synthesis in mitochondria. Further, from the results of recent studies on RRF of P. aeruginosa together with E. coli, it has been found that RRF is exchangeable between E. coli and P. aeruginosa. This fact may suggest that the inhibitor with broad antibiotic ability for prokaryotes can be developed through studies on E. coli RRF.

### SUMMARY OF THE INVENTION

An aspect of the present invention provides a method of developing an inhibitor of a ribosome recycling factor (RRF). The method comprises: providing data of a three-dimensional structure of RRF, the three-dimensional structure comprising a coiled-coil domain; identifying one or more amino acids of the coiled-coil domain that play a critical role in binding with ribosome A-site; obtaining a local three-dimensional structure in the vicinity of the one or more amino acids from the data of the three-dimensional structure of RRF; and designing or searching for substances comprising a three-dimensional structure, which is either substantially complimentary or substantially similar to the local three-dimensional structure in the vicinity of the one or more amino acids.

In the above-described method, the three-dimensional structure of RRF comprises a hydrophobic cleft, in which a compound is inserted. The method of claim 2, wherein at least one amino acids participates in hydrophobic binding with the compound, and wherein the at least one participating amino acids are selected from the group consisting of Thr-106, Arg-31, Pro-103, Leu-36, Leu-37, Ile-40, Leu-87, Leu-89 and Leu-102. The compound comprises 3-12 carbon straight-chain alkyl group. The compound further comprises a non-hydrophobic substituent group. The compound is decyl-β-D-maltopyranoside. The coiled-coil domain comprises three helices represented by SEQ ID NOS. 5-7 for Escherichia coli, SEQ ID NOS. 8-10 for Haemophilus influenzae, SEQ ID NOS. 11-13 for Psuedomonas aeuruginosa and SEQ ID NOS. 14-16 for Bacillus sublillis. The three-dimensional structure of RRF is obtained by X-ray crystallography of RRF.

In the above-described method, the provision of the three-dimensional structure of RRF comprises: providing an RRF; crystallizing the RRF in the presence of a detergent comprising a hydrophobic portion; and conducting X-ray diffraction analysis of the crystallized RRF combined with the detergent. The hydrophobic portion of the detergent is inserted into a cleft of the RRF. The one or more amino acids of the coiled-coil domain that play a critical role are selected from the group consisting of amino acid Nos. 1, 3, 4, 6, 7, 8, 10, 11, 12, 14, 15, 17, 18, 19, 107, 108, 109, 111, 112, 114, 115, 116, 118, 119, 120, 122, 123, 125, 126, 127, 129, 130, 132, 133, 134, 136, 137, 138, 140, 141, 143, 144, 145, 146, 147, 148, 150, 151, 152, 153, 155, 156, 158, 159, 161, 162, 164, 165, 166, 167, 169, 170, 172, 173, 174, 176, 177, 179, 180 and 181 in SEQ ID NOS. 1-4.

Still in the above-identified method, the identifying comprises: providing a microorganism comprising RRF; mutating the RRF of the microorganism by replacing one or more amino acids of the coiled-coil domain; observing growth of the microorganism with the mutated RRF; and determining whether the one or more amino acids replaced play a critical role in binding with a ribosome A-site from the observation of the growth. The designing or searching comprises conducting computerized modeling with the data of the three-dimensional structure of RRF. The method further comprises selecting a substance comprising a three-dimensional structure substantially complimentary or substantially similar to the local three-dimensional structure. The designing or searching further comprises modifying the selected substance. The method further comprises testing RRF inhibition activity of the selected substance. The testing is conducted in vitro or in vivo. The method further comprises synthesizing the selected substance. The synthesis is either biologically or chemically carried out. The substance is selected from the group consisting of amino acids, peptides, polypeptides and compounds comprising peptides.

Another aspect of the present invention provides a crystal of RRF. The crystal of RRF is the starting point for analyzing the three-dimensional structure of RRF, which is key information for the design, screening and identification of RRF inhibitors.

Another aspect of the present invention provides a method for a crystal of RRF or RRF homolog and its crystallization method. The crystal of RRF or RRF homolog can be obtained by crystallizing it with a material binding to a specific site of RRF. The crystal of RRF according to the present invention includes RRF and the material binding to the specific site of RRF. It is found that the specific site of RRF is a hydrophobic cleft formed between two domains in the three-dimensional structure of RRF analyzed by the obtained crystal of RRF according to the present invention. In the three-dimensional structure of RRF analyzed by the crystal of RRF according to the present invention, the two domains are formed with L-shape and the hydrophobic cleft is formed between the two domains. One domain of RRF can bind to ribosome and the activity of RRF may be exerted with the hydrophobic cleft.

Still another aspect of the present invention provides a method for identifying RRF inhibitors by means of the three-dimensional structure of RRF. By the present invention, RRF inhibitor candidates can be screened easily, and RRF inhibitors with optimum activity of RRF inhibition can be designed and identified. The method for identifying RRF inhibitors uses information from the three-dimensional structure of RRF.

Still another aspect of the present invention provides RRF inhibitors including maltopyranosides and their derivatives, which are identified as having the activity of RRF inhibition by the present invention.

A still further aspect of the present invention provides a polypeptide having a part or all of the structure of coiled coil domain of RRF. The polypeptide can inhibit the activity of RRF by competitively binding to the RRF binding site of ribosome, thereby preventing RRF from binding to ribosome. The polypeptide may include a part or all of the coiled coil domain of RRF.

### BRIEF DESCRIPTION OF THE DRAWINGS

The above and other objects and features of the present invention will become apparent from the following description of preferred embodiments given in conjunction with the accompanying drawings.

FIG. 1 provides a ribbon diagram of the three-dimensional structure of RRF analyzed with the crystal of RRF according to the present invention.

FIG. 2 provides a stick model showing a detergent molecule located in a cleft of RRF drawn by a surface model in the three-dimensional structure of RRF analyzed with the crystal of RRF according to the present invention.

FIG. 3 shows the multiple sequence alignment of RRFs derived from prokaryotes.

FIG. 4 shows relative movement between the two domains of the RRF in the three-dimensional structure of RRF analyzed with the crystal of RRF according to the present invention.

FIG. 5 provides the three-dimension structural surface models of RRF and molecules binding to A-site of ribosome such as tRNA and EF-G for showing the structural similarities therebetween.

### DETAILED DESCRIPTION OF THE PREFERRED EMBODIMENT

The invention will be illustrated in detail by the following preferred embodiments with reference to the accompanying drawings.

Abbreviations

“RRF” refers to ribosome recycling factor.

Abbreviations of Amino acids

| Amino acids | One Letter Symbol | Three Letter Symbol |
|---|---|---|
| Alanine | A | Ala |
| Arginine | R | Arg |
| Asparagine | N | Asn |
| Aspartic acid | D | Asp |
| Cysteine | C | Cys |
| Glutamine | Q | Gln |
| Glutamic acid | E | Glu |
| Glycine | G | Gly |
| Histidine | H | His |
| Isoleucine | I | Ile |
| Leucine | L | Leu |
| Lysine | K | Lys |
| Methionine | M | Met |
| Phenylalanine | F | Phe |
| Proline | P | Pro |
| Serine | S | Ser |
| Threonine | T | Thr |
| Tryptophan | W | Trp |
| Tyrosine | Y | Tyr |
| Valine | V | Val |

Definitions

The definitions used in this specification are as follows:

“Crystal” refers to protein of crystalline form. Crystal is to be piled up with several unit cells.

“Complex” refers to the form of protein binding with one or more materials.

“Unit cell” is the simplest and smallest volume of unit in a crystal. It is defined with a, b, c, alpha, beta and gamma (Blundell et al., 1976).

“Space group” is an arrangement of symmetric elements in a crystal. In the definition of space group, English capital letter refers to lattice type and other numeric refers to symmetric operator.

“Asymmetric Unit” refers to the largest unit that does not have symmetric element in the unit cell, but is superimposed with same shape by symmetric operation of space group.

“Multiple isomorphous replacement” is a method for obtaining information about phase for three-dimensional structure by using heavy metal derivatives made from infiltrating the heavy metal solution into a crystal (Blundell et al., 1976).

“Molecular replacement” is a method for obtaining a preliminary structure from crystal of unknown structure by changing the locations of molecules having known structure coordinates.

“RRF homologs” referred to in this application are meant to have at least 30% of amino acid similarity to RRF and include RRF mutants.

Crystallization of RRF and Structure of the Crystal

At first, the present invention is to obtain a crystal of RRF from a solution containing a precipitant such as polyethylene glycol with a detergent having hydrophobic part such as decyl-β-D-maltopyranoside. The crystal of RRF cannot be obtained in the state of being free of the detergent having hydrophobic part. As described below, this is caused by the fact that the three-dimensional structure of RRF consists of two domains, which move relative to each other, and thus it is difficult to crystallize RRF without the means for fixing the two domains. Prior to this invention, other research groups had tried to crystallize the isolated RRF from E. coli, but they did not succeed in the crystallization of RRF. Prior to this invention, we had also tried to crystallize the isolated RRF from E. coli in solutions with several concentrations of the protein under other crystallization conditions, but we did not succeed. It is considered that the failure for crystallization of RRF is caused from the fact that the crystallization was not performed with a detergent having hydrophobic part. The reason of necessity for the detergent in the crystallization of RRF is that as described below, the detergent binds to the cleft formed between the two domains of RRF, thereby eliminating the relative movement of the two domains. Therefore, in order to crystallize RRF or RRF mutant (or RRF analogy) isolated from E. coli or other bacteria, it is considered to be necessary of the material binding to the hydrophobic cleft of RRF as described below, preferably a detergent having hydrophobic part. Structure of RRF crystal

The RRF crystal obtained by the method as described above includes the detergent having hydrophobic part, decyl-β-D-maltopyranoside. The RRF crystal of the present invention may be analyzed with X-ray diffraction data to identify the three-dimensional structure of RRF. When being analyzed from the X-ray diffraction data, the RRF crystal of the present invention has a long stick shape with the unit cell of a=b=48.06 Å and c=142.27 Å, and the space group of P3₁21.

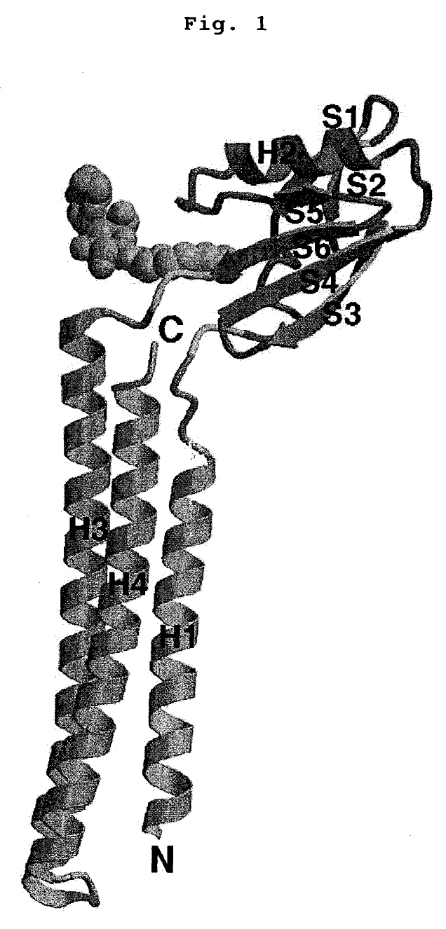

The three-dimensional structure of RRF is depicted in FIG. 1, which provides a ribbon diagram of the three-dimensional structure of RRF analyzed with the crystal of RRF according to the present invention. In FIG. 1, the two domains are linked with linkers, the arrows indicate strands and the cylinders indicate helices. The ends of amino acid and carboxylic acid are indicated respectively, and the detergent is depicted with a space filling model.

As shown in table 1, the three-dimensional structure of RRF was determined with 0.23 nm resolution to form the three-dimensional model for 1437 of protein atoms, 230 of water molecules and 16 atoms of decyl-β-D-maltopyronoside. The three-dimensional coordinate of each atom in the three-dimensional structure of RRF are shown in table 2. As shown in FIG. 1, RRF is an open L-shaped molecule comprising two domains. The N-terminal helix H1 of the first domain is linked with the second domain. Two antiparallel C-terminal α-helices H3 and H4 connected by a short U-turn are extended from the second domain and folded back to N-terminal helix H1. Thus, the first domain (coil domain) consists of a three-stranded antiparallel coiled-coil about 65 Å long, in which the seven-turn helix H1 packs into the parallel eleven-turn helix H3 and the antiparallel ten-turn helix H4. The two-turn overhang helices in the C-terminal end of H3 and the N-terminal end of H4 near the U-turn form a short two-stranded coiled-coil. The second domain (α/β domain) with approximate dimensions of 23×26×36 Å has an α/β topology containing a short β-hairpin (S1 and S2) and a single α-helix (H2) packed against one face of an antiparellel four-stranded β-sheet (S3-S6). Two domains are connected by two hinge residues (Arg31 and Pro103) with an approximate angle of 10° between the lines along helix H3 and strand S6.

TABLE 1
X-ray diffraction data and optimum result of RRF

| Data collection | |
|---|---|
| Resolution (Å) | 20.0-2.3 (2.38-2.30) |
| R_{merge} (%) | 4.8 (23.0) |
| Completeness (%) | 96.2 (97.7) |
| No. of unique reflections | 8,691 (858) |
| Redundancy | 3.34 |
| Refinement | |
| Resolution (Å) | 20.0-0.3 |
| R_{factor}/R_{free} (%) | 22.8/29.7 |
| No. of reflections (2 α cut off) | 8,391 |
| No. of solvent atoms | 230 |
| No. of protein atoms | 1437 |
| No. of Heteroatoms | 16 |
| Mean B factor (Å²) | 53.9 |
| R.m.s.d. in binding length (Å) | 0.010 |
| R.m.s.d in binding angle (°) | 1.63 |

The terms in the table are defined as follows:

“Resolution” is the distance between two plates when the diffraction conditions of Bragg are satisfied.

“R_{merge}” is the degree of accordance between measurements of diffraction intensities when they are measured repeatedly.

“R_{factor}” is the difference between the diffraction intensities obtained by experiment and calculated in the model.

“R_{free}” is R_{factor} calculated by using diffraction points excluded during optimum process in order to measure the usefulness of the obtained model.

Hydrophobic Cleft and Movement of Domains

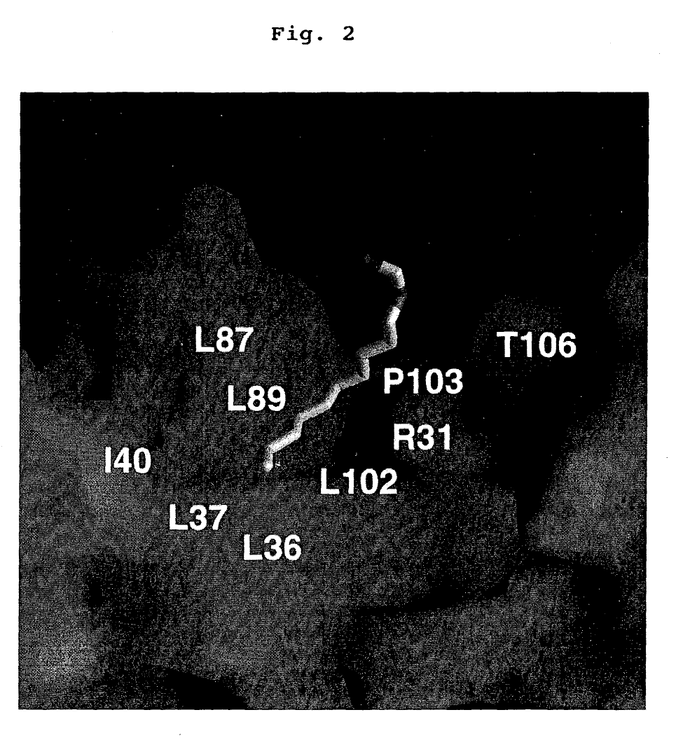

FIG. 2 provides a stick model showing a detergent located in a cleft of RRF drawn by surface model in the three-dimensional structure of RRF analyzed with the crystal of RRF according to the present invention. There are depicted amino acid residues involved in hydrophobic bonding with their names and location numbers (Thr-106, Arg-31, Pro-103, Leu-36, Leu-37, Ile-40, Leu-87, Leu-89, Leu-102) in the drawing. The head part of the detergent is not depicted, which is not involved in the hydrophobic bonding.

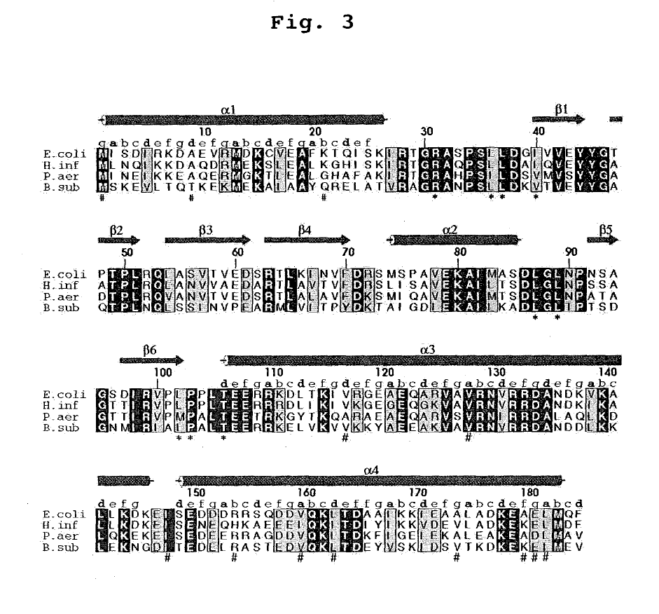

FIG. 3 shows multiple sequence alignment of RRFs derived from prokaryotes. There are used the sequences of RRFs derived from E. coli, Haemophilus influenzae (H. inf), Psuedomonas aeuruginosa (P. aeu) and Bacillus subtillis (B. sub) for multiple sequence alignment. The secondary structure of E. coli RRF is indicated by an arrow for a β-strand and by a cylinder for an α-helix. The amino acid residues that make contact with the alkyl chain of the detergent are marked with asterisks (*). The mutated residues identified in temperature-sensitive mutants of RRF (Janosi et al., 1998) are indicated by # marks. The heptad pattern (abc defg) of each residue in the coiled-coil is also shown. In four sequences, identical residues are boxed in black and homologous residues are boxed in gray.

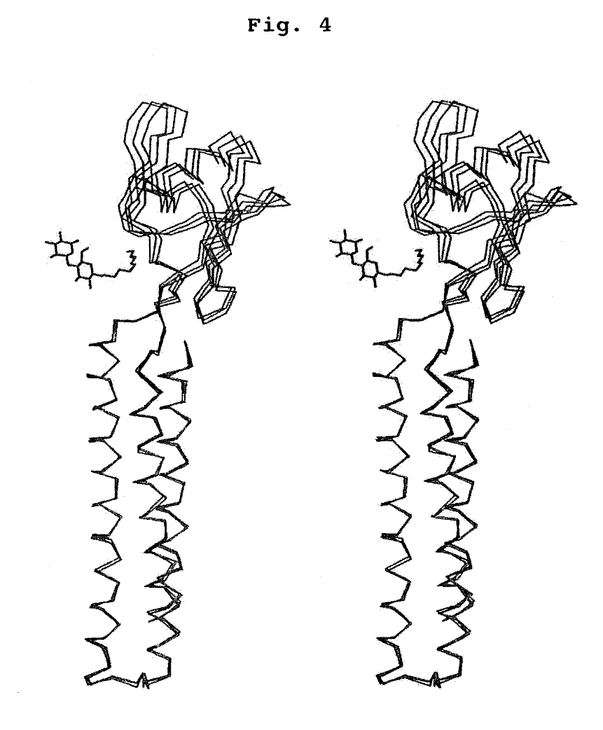

As shown in FIG. 2, the alkyl chain of the detergent, n-decyl-β-D-pyranomaltoside is located in the hydrophobic cleft formed at the joint between the two domains. Several hydrophobic amino acid residues form the hydrophobic cleft and interact hydrophobically with the alkyl chain of the detergent. As shown in FIG. 3, it is recognized from the comparisons of the protein sequences of several RRFs that the amino acid residues involved in binding the detergent are conserved very well. Therefore, it can be predicted easily that such amino acid residues are essential in binding other proteins or hydrophobic materials, and this binding is expected to be coupled with the activity of RRF. The contact of detergent in the hydrophobic cleft will decrease the relative movement between the two domains, which are linked through the cleft. Actually, the relative positions of the two domains are not the same in different crystals. This is shown in FIG. 4.

FIG. 4 shows relative movement between the two domains of the RRF in the three-dimensional structure of RRF analyzed with the crystal of RRF according to the present invention. When superimposing the lower domains in the structures of RRFs obtained from the three different crystals of RRFs, the movement of upper domains is observed. The three different crystals of RRFs are named of PT1 (left side), Native1 (center) and Native2 (right side). The detergent of Nativel is depicted in FIG. 4. Such movement would be more pronounced without the detergent. The relative movement of the two domains is a troublesome factor against crystallization of RRF itself, as described above.

It would be easily predicted that RRF binds with other molecules or their parts at the hydrophobic cleft formed between the relatively moving domains, in other words, the hydrophobic cleft plays a role of binding site to other molecules or their parts. This means that the hydrophobic cleft influences the function of RRF in the protein synthesis.

Meanwhile, the relative movement between the two domains of RRF and the hydrophobic cleft formed by the two domains of RRF are common phenomena discovered in the molecules binding to the A-site of ribosome such as tRNA and EF-G. This may support the fact that the relative movement between the two domains of RRF and the hydrophobic cleft is very important in relation to the role of protein synthesis.

Therefore, since RRF is an essential protein for viability of bacteria as referred above (Janosi et al., 1994), RRF is considered to be a main target for the development of antibacterial drug (Kaji et al., 1998) and one among the specific sites of RRF for the development of antibacterial drug will be the hydrophobic cleft as described above. Namely, since the material binding to the hydrophobic cleft of RRF blocks the movement between the two domains of RRF not to exert the function of RRF, the material can be easily screened and designed by using the three-dimensional structure of RRF according to the present invention. At the end, the material can be used as an RRF inhibitor, namely antibacterial agent.

The crystal of RRF according to the present invention is obtained from the complex of RRF with decyl-β-D-maltopyranoside. This shows that decyl-β-D-maltopyranoside is well bound to the hydrophobic cleft of RRF. Therefore, since decyl-β-D-maltopyranoside can inhibit the function of RRF, it can be used as an RRF inhibitor.

According to the three-dimensional structure of RRF analyzed from the crystal of RRF with the detergent, it is found that seven of carbon atoms on the alkyl chain of the detergent are bound to the hydrophobic cleft of RRF. Seven of the carbon atoms from the terminal of alkyl group are inserted into the hydrophobic cleft of RRF and the remaining three carbon atoms are out of the cleft. The maltose group (disaccharide of 6 carbon) does not interact with the non-hydrophobic residues in the circumference of the hydrophobic cleft. Therefore, the carbon number in the hydrophobic alkyl group of the detergent binding to the cleft of RRF is preferably about 3 to 12. When the carbon number is smaller, the hydrophobic bonding is very weak. When the carbon number is larger, the part of alkyl group not binding to the cleft of RRF may play a role of repulsion to the other part of RRF. It is preferable for the detergent to have at least one group that can do non-hydrophobic bonding (hydrogen bonding, dipole-dipole bonding, charge-charge bonding and so on) with non-hydrophobic residues in the circumference of or near to the hydrophobic cleft of RRF. In this material, the hydrophobic alkyl group plays a role of binding to the cleft of RRF, and the non-hydrophobic group can provide the specific bonding with the non-hydrophobic amino acid residues in the circumference of or near to the cleft of RRF. By these bonding, the material can maintain the contact to RRF with very strong affinity.

Therefore, the preferable structure of the detergent for inhibiting the function of RRF includes the alkyl group with 3˜12 of carbon atoms on the straight chain and non-hydrophobic group. The alkyl group of the detergent used in the present invention is preferably n-alkyl, but may be substituted with C1˜C3 alkyl group on at least one carbon of straight chain.

Coiled Coil Domain of RRF binding to A-site of Ribosome

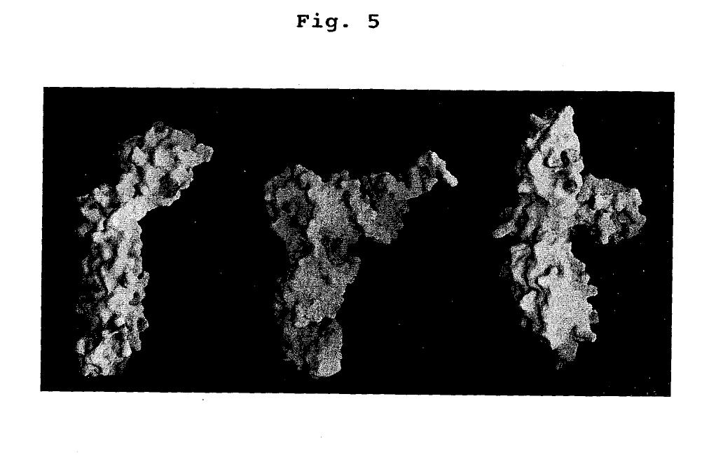

FIG. 5 provides the three-dimension structural surface models of RRF and molecules binding to A-site of ribosome such as tRNA and EF-G for showing the structural similarities therebetween. In the drawing, the left is RRF, the center is tRNA, and the right is 3,4,5 domains of EF-G. The white color indicates hydrophobic surface. The red and blue colors indicate negatively and positively charged surfaces, respectively.

As shown in FIG. 5, RRF is similar to the structures of tRNA and EF-G, which are molecules binding to A-site of ribosome. In particular, the domain corresponding to coiled coil of RRF has high structural similarity to anticodon stem of tRNA and the domain IV of EF-G. From these results and the other results of several experiments, it is considered that the site directly binding to A-site of ribosome is on the coiled coil domain of

Therefore, the specific site binding to A-site of ribosome on the coiled coil domain can be identified using the three-dimensional structure of RRF according to the present invention and the material binding to the specific site can be identified and designed. After all, since the material may prevent RRF from binding to A-site of ribosome, it can be used as an RRF inhibitor.

Identifying and Designing RRF Inhibitors by Using the Three-Dimensional Structure of RRF

1) Identifying and Designing RRF Inhibitors by Using Hydrophobic Cleft of RRF

The present invention relates to a method for identifying and designing the complementary material to the hydrophobic cleft of RRF by using the three-dimensional structure, and synthesizing it to use as an antibiotic. The complementary material to the cleft, namely, the material inhibiting the activity of RRF must be bound to the cleft of RRF physically and structurally. At this point, the binding is not covalent, but it includes hydrogen bonds, ionic bonds, van der Waals and hydrophobic interactions. The processes for identifying and designing RRF inhibitors are exemplified below.

a. The first step is to retrieve on database, the material or its part predictable to be complementary with the hydrophobic cleft of RRF in the three-dimensional structure or to design the material or its part based on the three-dimensional structure of the cleft. It is considered at this point that the material or its part must be bound to the cleft physically and structurally. The binding is not covalent, but may include hydrogen bonds, ion bonds, van der Waals and hydrophobic interactions. It is also considered that the compound or its part may be allowed to have a special conformation to bind the cleft. The conformation is not considered in the compound itself, but in the assumption of the compound binding with the cleft. Computer modeling can perform the selection and design of the compound or fragment. Further, prior to preparing the compound, the computer modeling technique can analyze or calculate the affinity between the compound or fragment and the cleft. If the computer modeling determines that the material or its part selected from database or designed has not sufficient affinity to the cleft, it is concluded not to have RRF inhibition ability and the subsequent processes are not performed, which include molecule remodeling for optimizing the affinity to the cleft such as modifying the structure of the compound and exchanging substituents, preparing the remodeled compound and measuring the activity of the prepared compound.

This process may increase the efficiency of developing RRF inhibitor, because it can exclude the preparation and activity measurement of the compound not to have the activity of inhibitor. If the computer modeling determines that the compound has the sufficient affinity to the cleft of RRF, it will be prepared and measured in vitro to have the activity as an inhibitor. At this point, if the complex of the compound-RRF is 100 micromoles or less in the dissociation constant, the compound may be considered as an inhibitor candidate. When the dissociation constant is 100 micromoles, 99% of the inhibitor exists in the complex and 1% of the remainder is dissociated from the complex. The less the dissociation constant is, the more the affinity of the compound to RRF is. There is described in the paragraph of b below, the design of the stronger inhibitor based on the inhibitor candidate. The process for calculating the affinity of the compound to RRF by the use of computer modeling technique is to enter the compound into the hydrophobic cleft of RRF with rotating it along several directions and to fit it to the cleft with visual inspection on the computer screen. This work may be performed with the modeling programs such as Insight II, Sybyl, O, Chain and Frodo. The method for inserting the compound into the hydrophobic cleft of RRF may be also performed by docking, which may be performed with the modeling programs such as Quanta, Autodock, Dock, Insight II, and Sybyl. The position of the compound located in the binding site through the process may be made to be more accurate by using energy minimization or molecular dynamics with standard molecular mechanics force fields such as CHARMM and AMBER.

Specialized computer programs may also need to perform the process of selecting the compound. Exemplary computer programs are as follows:

1. GRID (Goodford, P. J., “A Computational Procedure for Determining Energetically Favorable Binding Sites on Biologically Important Macromolecules”, J. Med. Chem., 28, pp. 849-857 (1985)). GRID is available from Oxford University, Oxford, UK.

2. MCSS (Miranker, A. and M. Karplus, “Functionality Maps of Binding Sites: A Multiple Copy Simultaneous Search Method” Proteins: Structure, Function and Genetics, 11, pp. 29-34 (1991)). MCSS is available from Molecular Simulations, Burlington, Mass.

3. AUTODOCK (Goodsell, D. S. and A. J. Olsen, “Automated Docking of Substates to Proteins by Simulated Annealing”, Proteins: Structure, Function and Genetics, 8, pp. 195-202 (1990)). AUTODOCK is available from Scripps Research Institue, La Jolla, Calif.

4. DOCK (Kuntz, I. D. et al., “A Geometric Approach to Macromolecule-Ligand Interactions”, J. Mol. Biol., 161, pp. 269-288 (1982). DOCK is available from University of California, San Fransisco, Calif.

When the suitable chemical compounds or fragments selected or designed are positioned in the binding site of the hydrophobic cleft and its circumferences, they can be assembled into a single compound. This may be performed by using the modeling programs as described above and the following programs:

1. CAVEAT (Bartlett, P. A. et al, “CAVEAT: A Program to Facilitate the Structure-Derived Design of Biologically Active Molecules” In “Molecular Recognition in Chemical and Biological Problems”, Special Pub., Royal Chem. Soc., 78, pp. 182-196 (1989). CAVEAT is available from the University of California, Berkeley, Calif.

2. 3D Database systems such as MACCS-3D (MDL Information Systems, San Leandro, Calif.). This area is reviewed in Martin, Y. C., “3D Database Searching in Drug Design”, J. Med. Chem., 35, pp. 2145-2154 (1992)).

3. HOOK (available from Molecular Simulations, Burlington, Mass.).

Once the material is designed or selected, the affinity of the material to RRF may be measured with calculation. The effective inhibitor must be low in the energy difference between those before and after binding. Thus, the most efficient inhibitor should preferably be designed with a deformation energy of binding of not greater than about 10 kcal/mole. The affinity of inhibitor may also be measured by calculating the dissociation constant of the complex of RRF and inhibitor. The dissociation constant is preferably 100 micromoles or less. The inhibitor must also maintain the bonding with RRF stably after binding. In order to do this, there are not the interactions of electrostatic repulsions such as charge-charge, dipole-dipole and charge-dipole interaction between the inhibitor and RRF. The sum of electrostatic interaction should be neutral or give a positive effect to the enthalpy of the bonding. Examples of programs designed for calculating such affinity are as follows: Gaussian 92, revision C [M. J. Frisch, Gaussian, Inc., Pittsburgh, Pa. © 1992]; AMBER, version 4.0 [P. A. Kollman, University of California at San Fransisco, © 1994]; QUANTA/CHARMM [Molecular Simulations, Inc., Burlington, Mass. © 1994]; and Insight II/Discover (Biosysm Technologies Inc., San Diego, Calif., ® 1994). Using the lead compound selected by the method, a stronger inhibitor can be made or designed. This process will be described below.

b. The compound (lead compound) selected or designed as above is changed or modified in atoms, substituents or a part of the structure to increase its affinity to RRF. Generally, initial substitutions are conservative, i.e., the replacement group will have approximately the same size, shape, hydrophobicity and charge as the original group. It should, of course, be understood that components known in the art to alter conformation should be avoided. Such substituted chemical compound may then be analyzed for efficiency of fit to RRF by the same computer methods described in detail, above.

Meanwhile, the present invention has identified the detergent, decyl-β-D-maltopyranoside, which has been used for obtaining RRF crystal, to bind to the hydrophobic cleft of RRF. So, this can be used as a lead compound for RRF inhibitors and performing the same computer methods described above. If needed, an appropriate substituent may be introduced into the hydrophobic chain, alkyl chain of decyl-β-D-maltopyranoside, or the maltose group may be changed into other appropriate groups.

c. After the material designed by the computer method described above is prepared and bound to RRF, the three-dimensional structure of the complex may be determined at high resolution (over 0.28 nm) using X-ray crystallographic method. The knowledge about the interaction between RRF and the inhibitor obtained from such structure may be used to modify the inhibitor and to increase the affinity of the inhibitor to RRF.

d. Once the inhibitor candidate for RRF optimized by such method has been selected or designed, the RRF inhibition activity of the candidate may be tested in vitro and in vivo. When the inhibitor candidate is determined to have the RRF inhibition activity, it may be used as an inhibitor or used for developing more optimized inhibitor based on its structure, for example, by improving its properties such as affinity to RRF and solubility using the computer modeling method. In other words, the compound may be used as a lead compound for computer remodeling to increase the properties of RRF inhibition activity, solubility and so on.

Before the compound is tested for inhibition activity, or regardless of the test, the dissociation constant of the complex of the inhibitor and RRF may be measured by actual experiment in order to identify the affinity calculated by the computer modeling method.

2) Identifying and Designing RRF Inhibitor Based on the Three-Dimensional Structure of the RRF Domain Binding to A-Site of Ribosome

a. Identifying essential amino acid residues needed to bind to ribosome in the coiled coil domain of RRF binding to A-site of ribosome. The locations of amino acid residues in the surface of the coiled coil domain are as follows: 1, 3, 4, 6, 7, 8, 10, 11, 12, 14, 15, 17, 18, 19, 107, 108, 109, 111, 112, 114, 115, 116, 118, 119, 120, 122, 123, 125, 126, 127, 129, 130, 132, 133, 134, 136, 137, 138, 140, 141, 143, 144, 145, 146, 147, 148, 150, 151, 152, 153, 155, 156, 158, 159, 161, 162, 164, 165, 166, 167, 169, 170, 172, 173, 174, 176, 177, 179, 180, and 181. The amino acid residues are identified on the three-dimensional structure of RRF (FIG. 1) and the coordinates of atoms in the structure of RRF (table 2). They are considered to be involved in binding to ribosome. The RRF can be changed into its mutants by exchanging one or more among the amino acid residues on the coiled coil domain into other amino acid residue(s). If E. coli possessing the mutant does not grow well or do slowly, the mutant may be determined to be a mutant without the function of RRF or with the decreased function of RRF. Namely, if so, the mutant cannot appropriately bind to ribosome due to the substitution of the amino acid residue, and thus cannot exert the function of RRF appropriately. Therefore, the amino acid residue in the original RRF substituted in the mutant of RRF will be an important amino acid residue in the binding of RRF to ribosome, and the site including the residue may be determined to be an important binding site of RRF to A-site of ribosome.

b. Identifying and designing an RRF inhibitor by using the structure of binding site in the coiled coil domain of RRF. Once some amino acid residues in the coiled coil domain of RRF have been identified as involving in binding to A-site of ribosome as described above, the structure of binding site including the residues can be recognized based on the three-dimensional structure of RRF. Then, based on the structure of binding site, the complementary material to the structure, for example peptide or other material can be screened and designed. For example, If the mutant including the residues substituted instead of Met-1, Ser-3 and Asp-4 would have the lowest activity, the three residues were found to be the most important residues in the binding of RRF to ribosome, and the complementary material to the structure of the binding site of RRF including the three residue would prevent RRF from binding to ribosome. The complementary material could be identified or designed from the structure of the binding site of RRF including the three residues.

The computer modeling method as described above can perform the identification and design of the complementary material. It can also perform the remodeling to improve the affinity or solubility of the identified or designed material by modifying the structure of it or substituting its substituents.

The optimized material may be then prepared and the inhibition activity of the material for RRF can be tested in vitro and in vivo. When the test confirms that the material has the inhibition activity against RRF, the material or its derivative can be used as an antibiotic. Using the method as described above, the compound identified to have the inhibition activity may be used as a lead compound to obtain the improved inhibitor.

In order to confirm the identified affinity from the computer modeling method before identifying the inhibition activity of the compound against RRF, the dissociation constant of the complex may be actually measured.

Identifying the Structure of Complex of Inhibitor and RRF or RRF Homolog by Using the Three-Dimensional Structure of RRF

The three-dimensional structure of RRF or its each domain can be used to identify other crystal forms of RRF, the crystal structures of RRF analogies (in case of having 30% of amino acid similarity or more to RRF), the mutants of RRF or the complex of RRF with other binding material. The molecular replacement can be used for this. In order to identify the structure of the proteins as described above by this method, the coordinates of E. coli RRF (table 2) should be used. This method can quickly identify the unknown crystal structure. The programs used in the method are as follows:

CNS (Brger, A. T., Adams, P. D., Clore, G. M., DeLano, W. L., Gros, P., Grosse-Kunstleve, R. W., Jiang, J. S., Kuszewski, J., Nilges, M., Pannu, N. S., Read, R. J., Rice, L. M., Simonson, T. and Warren, G. L. (1998), Crystallography & NMR system: A New software suite for macromolecular structure determination, Acta Crystallogr., D 54, 905-921).

CCP4 program package (Collaborative Computational Project Number 4 (1994), The CCP4 suite: programs for protein crystallography, Acta crystallogr., D 50, 760-763); It includes molecular replacement program of Amore, Polarrfn et al.

EPMR (Kissinger, C. R., Gehlhaar, D. K. and Fogel, D. B. (1999), Rapid automated molecular replacement by evolutionary search, Acta Crystallogr., D 55, 484-491).

Development for the Inhibitor Mimicking the Coiled Coil Domain of RRF

Since the coiled coil domain of RRF is to bind with A-site of ribosome, the polypeptide mimicking a part or all of the coiled coil domain of RRF can be bind with A-site of RRF. The polypeptide binds to ribosome competitively with RRF. As a result, it can prevent RRF from binding to ribosome, thereby being used as an inhibitor for RRF. The preparation of the polypeptide including the entire coiled coil domain of RRF may be easily performed using general biological technique or chemical synthetic technique based on the secondary structure of the amino acid sequence and the three-dimensional structure.

Meanwhile, although the peptide including the entire coiled coil domain of RRF can be used as an inhibitor for RRF, it is not considered to be efficient due to its large size. Therefore, it will be preferable that the inhibitor is an amino acid or polypeptide or material having its likely structure mimicking the three-dimensional structure of the binding site to ribosome in the coiled coil domain of RRF. As described above, the binding site to ribosome can be identified by testing the viability of E. coli containing the mutants at an amino acid residue on the surface of the coiled coil domain of RRF. The identification and design of the compound can be performed with the computer modeling method as described above. Further, for example, if a three-dimensional structure formed with two amino acids would be identified as a first binding part and another three-dimensional structure formed with three-amino acids is also identified as a second binding part near the first binding part, each of the polypeptides might be used as an inhibitor for RRF, respectively, and the compound formed by the appropriate link of one polypeptide with the other might be also used as an inhibitor for RRF. The latter compound would be of higher activity in the affinity to RRF than the former polypeptides.

In another method for developing the compound including an amino acid or polypeptide or material having its likely structure, for example, at first an amino acid or polypeptide or its likely material assumed as a binding part might be designed or synthesized. At this point, it is not known yet whether the part in the coiled coil domain is a binding part. Therefore, whether the compound actually has the inhibition activity against RRF will be identified by the test in vitro or in vivo. If the compound is identified to have the inhibition activity against RRF, it can be used as an inhibitor. It is also known from the above fact that the part having the same structure as the compound in the coiled coil domain is a binding part. Further, more as the described above, when two or more of the compounds including an amino acid or polypeptide are located closely to each other on the surface of the coiled coil domain, a new compound may be designed or synthesized by linking the amino acids or polypeptides. The new compound may then be tested to identify its inhibition activity against RRF. In the design of the linker linking the amino acids or polypeptides, if there are not significant errors, almost all of the new compounds will also have the inhibition activity against RRF. The compound linked with two or more of amino acids or polypeptide will exert stronger inhibition activity than the separate compounds, because it can bind tightly to ribosome.

### Examples

Expression of E. coli RRF and its Purification

E. coli RRF gene was amplified by PCR using E. coli genomic DNA as a template. The PCR product was restricted and ligated into an NdeI/XhoI-restricted pET22b vector (Novagen). The E.coli cells B834(DE3) transformed with pET22b-RRF plasmid were grown at 310 K in LB medium containing 50 μg ml⁻¹ ampicillin. The E. coli RRF was induced with 0.5 mM IPTG for 4 h. The cells were harvested at 277 K by centrifugation (5000 g for 6 min) and resuspended in buffer A (20 mM Tris-HCl pH 8.0, 10 mM MgCl₂, 1 mM PMSF) following sonication. After centrifugation for 30 min at 15000 g, the supernatant was applied onto a 5 ml HiTrap Q column (Pharmacia) equilibrated with buffer A. The flowthrough containing RRF was dialyzed against buffer B (20 mM NaH₂PO₄ pH 6.0, 10 mM MgCl₂) and loaded onto a 5 ml HiTrap SP column (Pharmacia) equilibrated with buffer B. The protein identified in the flowthrough was concentrated by ultrafiltration (Amicon, YM 10) and loaded onto a Superdex 200 gel-filtration column (HiLoad 16/60, Pharmacia) equilibrated with buffer A containing 150 mM NaCl. The fractions containing RRF were pooled and concentrated for crystallization. The purity of RRF was checked by SDS gel electrophoresis.

Crystallization and Structural Analysis of E. coli RRF

The crystal of E. coli RRF was made with polyethylene glycol as a precipitant in the presence of decyl-β-D-maltopyranoside as a detergent (Yun et al., 2000). The procedures are as follows: The protein was prepared at a concentration of 100 mg/ml in a buffer (20 mM Tris-HCl, pH 8.0, 10 mM MgCl₂, 1 mM PMSF, 150 mM NaCl). The crystal growth solution needed for crystal growth had the composition of 0.1 M MES-NaOH, pH 6.5, 10% PEG 350 MME, 12% PEG 400. After 2 μl of the prepared protein solution was taken on a cover glass to form about 2-3 mm diameter of a droplet, 2 μl of the crystal growth solution and 0.4 μl of 18 mM decyl-β-D-maltopyranoside were located on the droplet, in turn. There was not tried any action for mixing the three solutions except letting them alone for being naturally mixed by diffusion. The final droplet combined with the three solutions was sealed after the detergent was mixed, and equilibrated at 14° C. Vacuum grease was used for the sealing. The vapor diffusion of the solutions was begun in the closed system to equilibrate the solutions with the crystal growth solution and as a result, the concentration of the solution in the droplet was increased. Crystals were clustered and grew to dimensions of 0.15×0.15×1.0 mm within a week. When X-ray diffraction data from the crystal were collected and analyzed, it was found that this crystal had the unit cell of a=b=48.06 Å and c=142.27 Å, and the space group of P3₁21. It was also found from the results that the content of solvent was 53.4%, when one molecule was in an asymmetric unit. To interpret the structure, there were used multiple isomorphous replacement using anomalous scattering (MIRAS) and density modification such as solvent flipping. The programs of DENZO and SCALEPACK was used for treating the X-ray diffraction data, the program “O” for making the model, and the program “CNS” for optimizing the model.

TABLE 2
The coordinates of the three-dimensional structure of RRF
analyzed from the RRF crystal of the present invention

| Atom type | Number | Average B | Maximum B | Average Q |
|---|---|---|---|---|
| Protein main chain | 740 | 50.506 | 108.570 | 1.000 |
| Protein side chain | 697 | 54.907 | 111.870 | 1.000 |
| Protein all atoms | 1437 | 52.640 | 111.870 | 1.000 |
| Ligand/substrate | 0 | 0.000 | 0.000 | 0.000 |
| Water molecules | 231 | 61.297 | 96.420 | 1.000 |
| Other entities | 14 | 65.118 | 79.610 | 1.000 |
| All atoms | 1682 | 53.933 | 111.870 | 1.000 |

| Atom | No. | Atom Type | A.A. Type | A.A. No. | X | Y | Z | OCC | B |
|---|---|---|---|---|---|---|---|---|---|
| ATOM | 1 | CB | MET | 1 | 0.523 | 6.682 | 82.996 | 1.00 | 100.83 |
| ATOM | 2 | CG | MET | 1 | 0.660 | 5.557 | 84.005 | 1.00 | 102.28 |
| ATOM | 3 | SD | MET | 1 | −0.459 | 5.752 | 85.397 | 1.00 | 104.69 |
| ATOM | 4 | CE | MET | 1 | 0.064 | 7.342 | 86.019 | 1.00 | 104.89 |
| ATOM | 5 | C | MET | 1 | −0.879 | 7.980 | 81.395 | 1.00 | 98.25 |
| ATOM | 6 | O | MET | 1 | −1.129 | 7.768 | 80.208 | 1.00 | 98.22 |
| ATOM | 7 | N | MET | 1 | −1.317 | 5.554 | 81.781 | 1.00 | 98.73 |
| ATOM | 8 | CA | MET | 1 | −0.883 | 6.836 | 82.409 | 1.00 | 99.14 |
| ATOM | 9 | N | ILE | 2 | −0.586 | 9.186 | 81.876 | 1.00 | 97.02 |
| ATOM | 10 | CA | ILE | 2 | −0.537 | 10.381 | 81.035 | 1.00 | 95.89 |
| ATOM | 11 | CB | ILE | 2 | −0.478 | 11.655 | 81.900 | 1.00 | 95.52 |
| ATOM | 12 | CG2 | ILE | 2 | −0.335 | 12.882 | 81.031 | 1.00 | 95.12 |
| ATOM | 13 | CG1 | ILE | 2 | −1.750 | 11.762 | 82.734 | 1.00 | 95.60 |
| ATOM | 14 | CD1 | ILE | 2 | −1.825 | 13.021 | 83.549 | 1.00 | 95.83 |
| ATOM | 15 | C | ILE | 2 | 0.661 | 10.372 | 80.086 | 1.00 | 95.21 |
| ATOM | 16 | O | ILE | 2 | 0.655 | 11.047 | 79.058 | 1.00 | 95.10 |
| ATOM | 17 | N | SER | 3 | 1.689 | 9.609 | 80.442 | 1.00 | 94.28 |
| ATOM | 18 | CA | SER | 3 | 2.889 | 9.501 | 79.619 | 1.00 | 93.89 |
| ATOM | 19 | CB | SER | 3 | 3.943 | 8.643 | 80.329 | 1.00 | 94.52 |
| ATOM | 20 | OG | SER | 3 | 4.245 | 9.152 | 81.617 | 1.00 | 95.52 |
| ATOM | 21 | C | SER | 3 | 2.503 | 8.822 | 78.311 | 1.00 | 92.84 |
| ATOM | 22 | O | SER | 3 | 2.949 | 9.203 | 77.229 | 1.00 | 92.71 |
| ATOM | 23 | N | ASP | 4 | 1.658 | 7.808 | 78.439 | 1.00 | 91.48 |
| ATOM | 24 | CA | ASP | 4 | 1.182 | 7.025 | 77.310 | 1.00 | 90.84 |
| ATOM | 25 | CB | ASP | 4 | 0.514 | 5.751 | 77.837 | 1.00 | 92.24 |
| ATOM | 26 | CG | ASP | 4 | 1.366 | 5.036 | 78.880 | 1.00 | 94.26 |
| ATOM | 27 | OD1 | ASP | 4 | 0.921 | 3.995 | 79.408 | 1.00 | 96.07 |
| ATOM | 28 | OD2 | ASP | 4 | 2.482 | 5.516 | 79.184 | 1.00 | 95.50 |
| ATOM | 29 | C | ASP | 4 | 0.186 | 7.836 | 76.472 | 1.00 | 88.80 |
| ATOM | 30 | O | ASP | 4 | 0.469 | 8.209 | 75.331 | 1.00 | 89.03 |
| ATOM | 31 | N | ILE | 5 | −0.972 | 8.102 | 77.067 | 1.00 | 85.55 |
| ATOM | 32 | CA | ILE | 5 | −2.054 | 8.859 | 76.448 | 1.00 | 81.93 |
| ATOM | 33 | CB | ILE | 5 | −2.902 | 9.566 | 77.535 | 1.00 | 80.24 |
| ATOM | 34 | CG2 | ILE | 5 | −3.969 | 10.453 | 76.893 | 1.00 | 79.36 |
| ATOM | 35 | CG1 | ILE | 5 | −3.549 | 8.519 | 78.439 | 1.00 | 77.95 |
| ATOM | 36 | CD1 | ILE | 5 | −4.442 | 9.097 | 79.495 | 1.00 | 76.67 |
| ATOM | 37 | C | ILE | 5 | −1.686 | 9.895 | 75.379 | 1.00 | 81.10 |
| ATOM | 38 | O | ILE | 5 | −2.162 | 9.803 | 74.247 | 1.00 | 80.52 |
| ATOM | 39 | N | ARG | 6 | −0.857 | 10.879 | 75.718 | 1.00 | 79.50 |
| ATOM | 40 | CA | ARG | 6 | −0.514 | 11.909 | 74.739 | 1.00 | 79.11 |
| ATOM | 41 | CB | ARG | 6 | 0.158 | 13.107 | 75.403 | 1.00 | 80.27 |
| ATOM | 42 | CG | ARG | 6 | 1.465 | 12.784 | 76.077 | 1.00 | 83.34 |
| ATOM | 43 | CD | ARG | 6 | 2.301 | 14.039 | 76.196 | 1.00 | 86.04 |
| ATOM | 44 | NE | ARG | 6 | 2.611 | 14.574 | 74.871 | 1.00 | 87.69 |
| ATOM | 45 | CZ | ARG | 6 | 3.314 | 15.679 | 74.659 | 1.00 | 87.77 |
| ATOM | 46 | NH1 | ARG | 6 | 3.780 | 16.374 | 75.692 | 1.00 | 88.33 |
| ATOM | 47 | NH2 | ARG | 6 | 3.546 | 16.086 | 73.418 | 1.00 | 85.84 |
| ATOM | 48 | C | ARG | 6 | 0.377 | 11.424 | 73.605 | 1.00 | 77.92 |
| ATOM | 49 | O | ARG | 6 | 0.228 | 11.866 | 72.467 | 1.00 | 77.98 |
| ATOM | 50 | N | LYS | 7 | 1.317 | 10.538 | 73.913 | 1.00 | 76.33 |
| ATOM | 51 | CA | LYS | 7 | 2.209 | 10.015 | 72.886 | 1.00 | 74.77 |
| ATOM | 52 | CB | LYS | 7 | 3.382 | 9.262 | 73.525 | 1.00 | 75.87 |
| ATOM | 53 | CG | LYS | 7 | 4.265 | 10.122 | 74.433 | 1.00 | 75.59 |
| ATOM | 54 | CD | LYS | 7 | 4.940 | 11.252 | 73.667 | 1.00 | 75.59 |
| ATOM | 55 | CE | LYS | 7 | 5.786 | 12.101 | 74.604 | 1.00 | 75.85 |
| ATOM | 56 | NZ | LYS | 7 | 6.601 | 13.132 | 73.900 | 1.00 | 74.95 |
| ATOM | 57 | C | LYS | 7 | 1.373 | 9.078 | 72.029 | 1.00 | 72.71 |
| ATOM | 58 | O | LYS | 7 | 1.693 | 8.805 | 70.877 | 1.00 | 72.89 |
| ATOM | 59 | N | ASP | 8 | 0.286 | 8.603 | 72.618 | 1.00 | 70.58 |
| ATOM | 60 | CA | ASP | 8 | −0.646 | 7.714 | 71.950 | 1.00 | 69.48 |
| ATOM | 61 | CB | ASP | 8 | −1.552 | 7.075 | 73.005 | 1.00 | 70.59 |
| ATOM | 62 | CG | ASP | 8 | −2.393 | 5.940 | 72.459 | 1.00 | 72.83 |
| ATOM | 63 | OD1 | ASP | 8 | −3.070 | 6.132 | 71.424 | 1.00 | 72.86 |
| ATOM | 64 | OD2 | ASP | 8 | −2.390 | 4.852 | 73.086 | 1.00 | 72.96 |
| ATOM | 65 | C | ASP | 8 | −1.480 | 8.575 | 70.981 | 1.00 | 68.88 |
| ATOM | 66 | O | ASP | 8 | −1.787 | 8.167 | 69.859 | 1.00 | 68.57 |
| ATOM | 67 | N | ALA | 9 | −1.833 | 9.776 | 71.428 | 1.00 | 66.22 |
| ATOM | 68 | CA | ALA | 9 | −2.633 | 10.689 | 70.631 | 1.00 | 63.64 |
| ATOM | 69 | CB | ALA | 9 | −3.115 | 11.854 | 71.494 | 1.00 | 61.48 |
| ATOM | 70 | C | ALA | 9 | −1.885 | 11.215 | 69.413 | 1.00 | 62.96 |
| ATOM | 71 | O | ALA | 9 | −2.455 | 11.287 | 68.323 | 1.00 | 63.12 |
| ATOM | 72 | N | GLU | 10 | −0.617 | 11.580 | 69.575 | 1.00 | 61.05 |
| ATOM | 73 | CA | GLU | 10 | 0.124 | 12.114 | 68.437 | 1.00 | 60.56 |
| ATOM | 74 | CB | GLU | 10 | 1.385 | 12.848 | 68.888 | 1.00 | 63.50 |
| ATOM | 75 | CG | GLU | 10 | 2.407 | 11.994 | 69.599 | 1.00 | 67.84 |
| ATOM | 76 | CD | GLU | 10 | 3.728 | 12.731 | 69.774 | 1.00 | 71.48 |
| ATOM | 77 | OE1 | GLU | 10 | 4.344 | 13.089 | 68.737 | 1.00 | 72.15 |
| ATOM | 78 | OE2 | GLU | 10 | 4.141 | 12.958 | 70.938 | 1.00 | 73.41 |
| ATOM | 79 | C | GLU | 10 | 0.487 | 11.041 | 67.422 | 1.00 | 57.27 |
| ATOM | 80 | O | GLU | 10 | 0.727 | 11.338 | 66.255 | 1.00 | 55.22 |
| ATOM | 81 | N | VAL | 11 | 0.549 | 9.794 | 67.863 | 1.00 | 55.19 |
| ATOM | 82 | CA | VAL | 11 | 0.839 | 8.728 | 66.922 | 1.00 | 53.74 |
| ATOM | 83 | CB | VAL | 11 | 1.232 | 7.412 | 67.629 | 1.00 | 54.76 |
| ATOM | 84 | CG1 | VAL | 11 | 1.279 | 6.261 | 66.616 | 1.00 | 54.05 |
| ATOM | 85 | CG2 | VAL | 11 | 2.608 | 7.576 | 68.265 | 1.00 | 54.99 |
| ATOM | 86 | C | VAL | 11 | −0.442 | 8.545 | 66.119 | 1.00 | 50.95 |
| ATOM | 87 | O | VAL | 11 | −0.408 | 8.228 | 64.935 | 1.00 | 51.54 |
| ATOM | 88 | N | ARG | 12 | −1.574 | 8.760 | 66.772 | 1.00 | 47.29 |
| ATOM | 89 | CA | ARG | 12 | −2.842 | 8.663 | 66.088 | 1.00 | 45.50 |
| ATOM | 90 | CB | ARG | 12 | −3.978 | 8.633 | 67.112 | 1.00 | 43.97 |
| ATOM | 91 | CG | ARG | 12 | −4.126 | 7.256 | 67.793 | 1.00 | 45.79 |
| ATOM | 92 | CD | ARG | 12 | −5.102 | 7.294 | 68.965 | 1.00 | 44.31 |
| ATOM | 93 | NE | ARG | 12 | −5.421 | 5.978 | 69.509 | 1.00 | 44.84 |
| ATOM | 94 | CZ | ARG | 12 | −6.083 | 5.776 | 70.653 | 1.00 | 43.11 |
| ATOM | 95 | NH1 | ARG | 12 | −6.492 | 6.794 | 71.379 | 1.00 | 42.34 |
| ATOM | 96 | NH2 | ARG | 12 | −6.339 | 4.550 | 71.080 | 1.00 | 45.09 |
| ATOM | 97 | C | ARG | 12 | −2.967 | 9.838 | 65.089 | 1.00 | 43.22 |
| ATOM | 98 | O | ARG | 12 | −3.217 | 9.627 | 63.908 | 1.00 | 40.86 |
| ATOM | 99 | N | MET | 13 | −2.732 | 11.063 | 65.543 | 1.00 | 43.08 |
| ATOM | 100 | CA | MET | 13 | −2.818 | 12.217 | 64.651 | 1.00 | 44.05 |
| ATOM | 101 | CB | MET | 13 | −2.617 | 13.519 | 65.421 | 1.00 | 43.78 |
| ATOM | 102 | CG | MET | 13 | −3.705 | 13.778 | 66.433 | 1.00 | 41.76 |
| ATOM | 103 | SD | MET | 13 | −3.401 | 15.313 | 67.300 | 1.00 | 47.04 |
| ATOM | 104 | CE | MET | 13 | −4.627 | 15.149 | 68.684 | 1.00 | 41.70 |
| ATOM | 105 | C | MET | 13 | −1.848 | 12.179 | 63.482 | 1.00 | 43.79 |
| ATOM | 106 | O | MET | 13 | −2.173 | 12.658 | 62.402 | 1.00 | 42.68 |
| ATOM | 107 | N | ASP | 14 | −0.662 | 11.619 | 63.700 | 1.00 | 45.44 |
| ATOM | 108 | CA | ASP | 14 | 0.337 | 11.507 | 62.641 | 1.00 | 47.26 |
| ATOM | 109 | CB | ASP | 14 | 1.665 | 10.990 | 63.209 | 1.00 | 52.47 |
| ATOM | 110 | CG | ASP | 14 | 2.658 | 12.101 | 63.429 | 1.00 | 55.85 |
| ATOM | 111 | OD1 | ASP | 14 | 3.764 | 11.830 | 63.938 | 1.00 | 60.98 |
| ATOM | 112 | OD2 | ASP | 14 | 2.331 | 13.254 | 63.075 | 1.00 | 57.16 |
| ATOM | 113 | C | ASP | 14 | −0.151 | 10.586 | 61.540 | 1.00 | 45.15 |
| ATOM | 114 | O | ASP | 14 | −0.008 | 10.867 | 60.358 | 1.00 | 45.21 |
| ATOM | 115 | N | LYS | 15 | −0.733 | 9.474 | 61.945 | 1.00 | 45.09 |
| ATOM | 116 | CA | LYS | 15 | −1.264 | 8.529 | 60.999 | 1.00 | 45.94 |
| ATOM | 117 | CB | LYS | 15 | −1.884 | 7.365 | 61.763 | 1.00 | 45.00 |
| ATOM | 118 | CG | LYS | 15 | −2.329 | 6.210 | 60.901 | 1.00 | 49.99 |
| ATOM | 119 | CD | LYS | 15 | −1.157 | 5.411 | 60.295 | 1.00 | 51.63 |
| ATOM | 120 | CE | LYS | 15 | −0.326 | 4.634 | 61.339 | 1.00 | 52.42 |
| ATOM | 121 | NZ | LYS | 15 | 0.435 | 5.471 | 62.309 | 1.00 | 52.22 |
| ATOM | 122 | C | LYS | 15 | −2.310 | 9.253 | 60.118 | 1.00 | 46.27 |
| ATOM | 123 | O | LYS | 15 | −2.313 | 9.094 | 58.889 | 1.00 | 48.65 |
| ATOM | 124 | N | CYS | 16 | −3.173 | 10.067 | 60.734 | 1.00 | 44.90 |
| ATOM | 125 | CA | CYS | 16 | −4.200 | 10.809 | 59.985 | 1.00 | 44.28 |
| ATOM | 126 | CB | CYS | 16 | −5.057 | 11.683 | 60.916 | 1.00 | 43.61 |
| ATOM | 127 | SG | CYS | 16 | −6.022 | 10.834 | 62.181 | 1.00 | 53.86 |
| ATOM | 128 | C | CYS | 16 | −3.604 | 11.730 | 58.908 | 1.00 | 41.26 |
| ATOM | 129 | O | CYS | 16 | −3.971 | 11.676 | 57.729 | 1.00 | 40.04 |
| ATOM | 130 | N | VAL | 17 | −2.697 | 12.596 | 59.324 | 1.00 | 38.60 |
| ATOM | 131 | CA | VAL | 17 | −2.091 | 13.512 | 58.384 | 1.00 | 38.03 |
| ATOM | 132 | CB | VAL | 17 | −1.082 | 14.435 | 59.092 | 1.00 | 39.03 |
| ATOM | 133 | CG1 | VAL | 17 | −0.512 | 15.452 | 58.111 | 1.00 | 38.47 |
| ATOM | 134 | CG2 | VAL | 17 | −1.766 | 15.144 | 60.257 | 1.00 | 38.78 |
| ATOM | 135 | C | VAL | 17 | −1.394 | 12.737 | 57.269 | 1.00 | 38.04 |
| ATOM | 136 | O | VAL | 17 | −1.530 | 13.074 | 56.095 | 1.00 | 35.00 |
| ATOM | 137 | N | GLU | 18 | −0.670 | 11.684 | 57.635 | 1.00 | 37.93 |
| ATOM | 138 | CA | GLU | 18 | 0.034 | 10.913 | 56.636 | 1.00 | 42.29 |
| ATOM | 139 | CB | GLU | 18 | 1.057 | 9.962 | 57.315 | 1.00 | 47.92 |
| ATOM | 140 | CG | GLU | 18 | 2.136 | 10.774 | 58.105 | 1.00 | 55.13 |
| ATOM | 141 | CD | GLU | 18 | 3.403 | 9.987 | 58.501 | 1.00 | 60.44 |
| ATOM | 142 | OE1 | GLU | 18 | 4.048 | 9.378 | 57.613 | 1.00 | 60.51 |
| ATOM | 143 | OE2 | GLU | 18 | 3.779 | 10.009 | 59.704 | 1.00 | 62.88 |
| ATOM | 144 | C | GLU | 18 | −0.935 | 10.205 | 55.687 | 1.00 | 41.27 |
| ATOM | 145 | O | GLU | 18 | −0.668 | 10.128 | 54.493 | 1.00 | 43.84 |
| ATOM | 146 | N | ALA | 19 | −2.072 | 9.726 | 56.187 | 1.00 | 38.68 |
| ATOM | 147 | CA | ALA | 19 | −3.060 | 9.110 | 55.300 | 1.00 | 37.55 |
| ATOM | 148 | CB | ALA | 19 | −4.241 | 8.521 | 56.116 | 1.00 | 35.72 |
| ATOM | 149 | C | ALA | 19 | −3.580 | 10.195 | 54.327 | 1.00 | 36.85 |
| ATOM | 150 | O | ALA | 19 | −3.802 | 9.937 | 53.133 | 1.00 | 39.34 |
| ATOM | 151 | N | PHE | 20 | −3.773 | 11.404 | 54.838 | 1.00 | 32.59 |
| ATOM | 152 | CA | PHE | 20 | −4.234 | 12.498 | 54.010 | 1.00 | 33.05 |
| ATOM | 153 | CB | PHE | 20 | −4.528 | 13.725 | 54.888 | 1.00 | 33.08 |
| ATOM | 154 | CG | PHE | 20 | −4.785 | 14.995 | 54.111 | 1.00 | 32.56 |
| ATOM | 155 | CD1 | PHE | 20 | −5.943 | 15.148 | 53.355 | 1.00 | 32.24 |
| ATOM | 156 | CD2 | PHE | 20 | −3.868 | 16.037 | 54.148 | 1.00 | 30.14 |
| ATOM | 157 | CE1 | PHE | 20 | −6.183 | 16.340 | 52.640 | 1.00 | 34.71 |
| ATOM | 158 | CE2 | PHE | 20 | −4.094 | 17.219 | 53.447 | 1.00 | 34.68 |
| ATOM | 159 | CZ | PHE | 20 | −5.257 | 17.375 | 52.689 | 1.00 | 32.40 |
| ATOM | 160 | C | PHE | 20 | −3.162 | 12.830 | 52.965 | 1.00 | 35.46 |
| ATOM | 161 | O | PHE | 20 | −3.460 | 13.017 | 51.772 | 1.00 | 36.29 |
| ATOM | 162 | N | LYS | 21 | −1.911 | 12.916 | 53.405 | 1.00 | 36.65 |
| ATOM | 163 | CA | LYS | 21 | −0.839 | 13.204 | 52.469 | 1.00 | 39.85 |
| ATOM | 164 | CB | LYS | 21 | 0.494 | 13.397 | 53.205 | 1.00 | 40.00 |
| ATOM | 165 | CG | LYS | 21 | 0.540 | 14.700 | 53.984 | 1.00 | 42.94 |
| ATOM | 166 | CD | LYS | 21 | 1.873 | 14.968 | 54.666 | 1.00 | 46.52 |
| ATOM | 167 | CE | LYS | 21 | 2.078 | 14.136 | 55.922 | 1.00 | 51.70 |
| ATOM | 168 | NZ | LYS | 21 | 2.091 | 12.659 | 55.699 | 1.00 | 56.00 |
| ATOM | 169 | C | LYS | 21 | −0.733 | 12.133 | 51.372 | 1.00 | 42.04 |
| ATOM | 170 | O | LYS | 21 | −0.480 | 12.477 | 50.221 | 1.00 | 42.08 |
| ATOM | 171 | N | THR | 22 | −0.966 | 10.855 | 51.686 | 1.00 | 44.06 |
| ATOM | 172 | CA | THR | 22 | −0.873 | 9.854 | 50.620 | 1.00 | 47.37 |
| ATOM | 173 | CB | THR | 22 | −0.619 | 8.362 | 51.126 | 1.00 | 46.50 |
| ATOM | 174 | OG1 | THR | 22 | −1.792 | 7.832 | 51.754 | 1.00 | 48.78 |
| ATOM | 175 | CG2 | THR | 22 | 0.524 | 8.314 | 52.105 | 1.00 | 45.25 |
| ATOM | 176 | C | THR | 22 | −2.141 | 9.863 | 49.787 | 1.00 | 47.95 |
| ATOM | 177 | O | THR | 22 | −2.091 | 9.686 | 48.569 | 1.00 | 50.97 |
| ATOM | 178 | N | GLN | 23 | −3.281 | 10.086 | 50.423 | 1.00 | 50.33 |
| ATOM | 179 | CA | GLN | 23 | −4.540 | 10.085 | 49.683 | 1.00 | 52.74 |
| ATOM | 180 | CB | GLN | 23 | −5.705 | 10.316 | 50.637 | 1.00 | 55.39 |
| ATOM | 181 | CG | GLN | 23 | −7.076 | 9.998 | 50.056 | 1.00 | 59.39 |
| ATOM | 182 | CD | GLN | 23 | −8.213 | 10.215 | 51.056 | 1.00 | 61.59 |
| ATOM | 183 | OE1 | GLN | 23 | −9.377 | 9.920 | 50.761 | 1.00 | 63.37 |
| ATOM | 184 | NE2 | GLN | 23 | −7.881 | 10.734 | 52.239 | 1.00 | 60.88 |
| ATOM | 185 | C | GLN | 23 | −4.549 | 11.159 | 48.598 | 1.00 | 55.03 |
| ATOM | 186 | O | GLN | 23 | −5.064 | 10.948 | 47.487 | 1.00 | 56.03 |
| ATOM | 187 | N | ILE | 24 | −3.946 | 12.299 | 48.923 | 1.00 | 54.26 |
| ATOM | 188 | CA | ILE | 24 | −3.891 | 13.443 | 48.032 | 1.00 | 54.96 |
| ATOM | 189 | CB | ILE | 24 | −3.712 | 14.733 | 48.888 | 1.00 | 56.27 |
| ATOM | 190 | CG2 | ILE | 24 | −2.378 | 14.711 | 49.601 | 1.00 | 57.16 |
| ATOM | 191 | CG1 | ILE | 24 | −3.785 | 15.976 | 48.019 | 1.00 | 56.46 |
| ATOM | 192 | CD1 | ILE | 24 | −3.556 | 17.244 | 48.803 | 1.00 | 57.81 |
| ATOM | 193 | C | ILE | 24 | −2.821 | 13.376 | 46.917 | 1.00 | 55.89 |
| ATOM | 194 | O | ILE | 24 | −3.043 | 13.870 | 45.808 | 1.00 | 53.59 |
| ATOM | 195 | N | SER | 25 | −1.672 | 12.759 | 47.188 | 1.00 | 56.28 |
| ATOM | 196 | CA | SER | 25 | −0.621 | 12.682 | 46.168 | 1.00 | 57.70 |
| ATOM | 197 | CB | SER | 25 | 0.647 | 12.031 | 46.734 | 1.00 | 56.60 |
| ATOM | 198 | OG | SER | 25 | 0.421 | 10.671 | 47.061 | 1.00 | 58.84 |
| ATOM | 199 | C | SER | 25 | −1.088 | 11.911 | 44.934 | 1.00 | 57.90 |
| ATOM | 200 | O | SER | 25 | −0.480 | 12.007 | 43.868 | 1.00 | 59.60 |
| ATOM | 201 | N | LYS | 26 | −2.167 | 11.147 | 45.081 | 1.00 | 57.63 |
| ATOM | 202 | CA | LYS | 26 | −2.723 | 10.384 | 43.975 | 1.00 | 57.26 |
| ATOM | 203 | CB | LYS | 26 | −3.825 | 9.454 | 44.474 | 1.00 | 58.98 |
| ATOM | 204 | CG | LYS | 26 | −3.353 | 8.353 | 45.412 | 1.00 | 62.43 |
| ATOM | 205 | CD | LYS | 26 | −4.504 | 7.416 | 45.750 | 1.00 | 64.95 |
| ATOM | 206 | CE | LYS | 26 | −4.044 | 6.265 | 46.633 | 1.00 | 67.37 |
| ATOM | 207 | NZ | LYS | 26 | −2.998 | 5.431 | 45.960 | 1.00 | 68.34 |
| ATOM | 208 | C | LYS | 26 | −3.303 | 11.297 | 42.899 | 1.00 | 57.18 |
| ATOM | 209 | O | LYS | 26 | −3.256 | 10.982 | 41.709 | 1.00 | 58.16 |
| ATOM | 210 | N | ILE | 27 | −3.845 | 12.431 | 43.330 | 1.00 | 55.61 |
| ATOM | 211 | CA | ILE | 27 | −4.457 | 13.411 | 42.436 | 1.00 | 54.60 |
| ATOM | 212 | CB | ILE | 27 | −5.180 | 14.523 | 43.250 | 1.00 | 54.29 |
| ATOM | 213 | CG2 | ILE | 27 | −5.451 | 15.726 | 42.360 | 1.00 | 54.06 |
| ATOM | 214 | CG1 | ILE | 27 | −6.456 | 13.967 | 43.878 | 1.00 | 51.10 |
| ATOM | 215 | CD1 | ILE | 27 | −6.215 | 12.818 | 44.784 | 1.00 | 49.52 |
| ATOM | 216 | C | ILE | 27 | −3.515 | 14.096 | 41.444 | 1.00 | 54.74 |
| ATOM | 217 | O | ILE | 27 | −2.557 | 14.776 | 41.825 | 1.00 | 52.96 |
| ATOM | 218 | N | ARG | 28 | −3.810 | 13.925 | 40.163 | 1.00 | 54.44 |
| ATOM | 219 | CA | ARG | 28 | −3.011 | 14.551 | 39.133 | 1.00 | 54.79 |
| ATOM | 220 | CB | ARG | 28 | −3.040 | 13.701 | 37.856 | 1.00 | 57.53 |
| ATOM | 221 | CG | ARG | 28 | −2.381 | 12.330 | 38.025 | 1.00 | 58.98 |
| ATOM | 222 | CD | ARG | 28 | −2.325 | 11.578 | 36.707 | 1.00 | 63.90 |
| ATOM | 223 | NE | ARG | 28 | −1.576 | 10.328 | 36.823 | 1.00 | 68.44 |
| ATOM | 224 | CZ | ARG | 28 | −1.298 | 9.509 | 35.808 | 1.00 | 69.24 |
| ATOM | 225 | NH1 | ARG | 28 | −1.709 | 9.801 | 34.575 | 1.00 | 69.86 |
| ATOM | 226 | NH2 | ARG | 28 | −0.608 | 8.394 | 36.027 | 1.00 | 67.68 |
| ATOM | 227 | C | ARG | 28 | −3.578 | 15.949 | 38.893 | 1.00 | 54.14 |
| ATOM | 228 | O | ARG | 28 | −4.795 | 16.154 | 38.889 | 1.00 | 55.27 |
| ATOM | 229 | N | THR | 29 | −2.698 | 16.915 | 38.705 | 1.00 | 51.89 |
| ATOM | 230 | CA | THR | 29 | −3.134 | 18.285 | 38.506 | 1.00 | 51.42 |
| ATOM | 231 | CB | THR | 29 | −2.414 | 19.187 | 39.502 | 1.00 | 51.85 |
| ATOM | 232 | OG1 | THR | 29 | −1.002 | 19.027 | 39.344 | 1.00 | 51.16 |
| ATOM | 233 | CG2 | THR | 29 | −2.786 | 18.798 | 40.924 | 1.00 | 51.25 |
| ATOM | 234 | C | THR | 29 | −2.918 | 18.810 | 37.082 | 1.00 | 50.96 |
| ATOM | 235 | O | THR | 29 | −3.337 | 19.926 | 36.752 | 1.00 | 50.77 |
| ATOM | 236 | N | GLY | 30 | −2.269 | 17.997 | 36.247 | 1.00 | 49.33 |
| ATOM | 237 | CA | GLY | 30 | −2.007 | 18.372 | 34.863 | 1.00 | 46.49 |
| ATOM | 238 | C | GLY | 30 | −2.275 | 17.181 | 33.963 | 1.00 | 44.01 |
| ATOM | 239 | O | GLY | 30 | −2.493 | 16.082 | 34.461 | 1.00 | 43.52 |
| ATOM | 240 | N | ARG | 31 | −2.256 | 17.386 | 32.649 | 1.00 | 42.12 |
| ATOM | 241 | CA | ARG | 31 | −2.520 | 16.301 | 31.703 | 1.00 | 40.50 |
| ATOM | 242 | CB | ARG | 31 | −2.980 | 16.873 | 30.361 | 1.00 | 41.80 |
| ATOM | 243 | CG | ARG | 31 | −4.281 | 17.585 | 30.412 | 1.00 | 41.56 |
| ATOM | 244 | CD | ARG | 31 | −4.707 | 18.090 | 29.040 | 1.00 | 42.11 |
| ATOM | 245 | NE | ARG | 31 | −6.018 | 18.713 | 29.146 | 1.00 | 42.16 |
| ATOM | 246 | CZ | ARG | 31 | −7.148 | 18.040 | 29.339 | 1.00 | 42.39 |
| ATOM | 247 | NH1 | ARG | 31 | −7.144 | 16.718 | 29.439 | 1.00 | 40.83 |
| ATOM | 248 | NH2 | ARG | 31 | −8.279 | 18.699 | 29.490 | 1.00 | 43.78 |
| ATOM | 249 | C | ARG | 31 | −1.350 | 15.347 | 31.440 | 1.00 | 38.97 |
| ATOM | 250 | O | ARG | 31 | −1.540 | 14.209 | 31.015 | 1.00 | 41.05 |
| ATOM | 251 | N | ALA | 32 | −0.143 | 15.812 | 31.698 | 1.00 | 37.59 |
| ATOM | 252 | CA | ALA | 32 | 1.059 | 15.033 | 31.454 | 1.00 | 36.56 |
| ATOM | 253 | CB | ALA | 32 | 2.278 | 15.811 | 31.987 | 1.00 | 36.73 |
| ATOM | 254 | C | ALA | 32 | 1.136 | 13.585 | 31.934 | 1.00 | 34.78 |
| ATOM | 255 | O | ALA | 32 | 0.995 | 13.299 | 33.114 | 1.00 | 37.37 |
| ATOM | 256 | N | SER | 33 | 1.397 | 12.678 | 31.001 | 1.00 | 34.58 |
| ATOM | 257 | CA | SER | 33 | 1.589 | 11.245 | 31.291 | 1.00 | 34.06 |
| ATOM | 258 | CB | SER | 33 | 0.253 | 10.507 | 31.463 | 1.00 | 36.06 |
| ATOM | 259 | OG | SER | 33 | 0.408 | 9.125 | 31.146 | 1.00 | 37.00 |
| ATOM | 260 | C | SER | 33 | 2.336 | 10.622 | 30.122 | 1.00 | 32.37 |
| ATOM | 261 | O | SER | 33 | 2.196 | 11.073 | 29.008 | 1.00 | 31.06 |
| ATOM | 262 | N | PRO | 34 | 3.192 | 9.621 | 30.377 | 1.00 | 32.68 |
| ATOM | 263 | CD | PRO | 34 | 3.664 | 9.098 | 31.670 | 1.00 | 33.36 |
| ATOM | 264 | CA | PRO | 34 | 3.924 | 8.970 | 29.285 | 1.00 | 34.44 |
| ATOM | 265 | CB | PRO | 34 | 4.871 | 8.021 | 30.029 | 1.00 | 34.92 |
| ATOM | 266 | CG | PRO | 34 | 4.085 | 7.690 | 31.284 | 1.00 | 34.26 |
| ATOM | 267 | C | PRO | 34 | 3.043 | 8.246 | 28.242 | 1.00 | 35.65 |
| ATOM | 268 | O | PRO | 34 | 3.463 | 8.094 | 27.097 | 1.00 | 36.81 |
| ATOM | 269 | N | SER | 35 | 1.829 | 7.824 | 28.613 | 1.00 | 34.52 |
| ATOM | 270 | CA | SER | 35 | 0.955 | 7.135 | 27.657 | 1.00 | 34.26 |
| ATOM | 271 | CB | SER | 35 | 0.267 | 5.925 | 28.308 | 1.00 | 33.31 |
| ATOM | 272 | OG | SER | 35 | −0.716 | 6.322 | 29.254 | 1.00 | 36.98 |
| ATOM | 273 | C | SER | 35 | −0.137 | 8.015 | 27.045 | 1.00 | 34.89 |
| ATOM | 274 | O | SER | 35 | −0.975 | 7.514 | 26.308 | 1.00 | 35.76 |
| ATOM | 275 | N | LEU | 36 | −0.120 | 9.313 | 27.334 | 1.00 | 34.17 |
| ATOM | 276 | CA | LEU | 36 | −1.148 | 10.248 | 26.835 | 1.00 | 34.56 |
| ATOM | 277 | CB | LEU | 36 | −0.771 | 11.700 | 27.195 | 1.00 | 35.25 |
| ATOM | 278 | CG | LEU | 36 | −1.881 | 12.732 | 26.941 | 1.00 | 33.58 |
| ATOM | 279 | CD1 | LEU | 36 | −3.008 | 12.435 | 27.923 | 1.00 | 35.46 |
| ATOM | 280 | CD2 | LEU | 36 | −1.383 | 14.168 | 27.139 | 1.00 | 33.44 |
| ATOM | 281 | C | LEU | 36 | −1.426 | 10.201 | 25.332 | 1.00 | 32.30 |
| ATOM | 282 | O | LEU | 36 | −2.562 | 10.295 | 24.891 | 1.00 | 29.65 |
| ATOM | 283 | N | LEU | 37 | −0.369 | 10.063 | 24.548 | 1.00 | 32.28 |
| ATOM | 284 | CA | LEU | 37 | −0.500 | 10.055 | 23.103 | 1.00 | 30.09 |
| ATOM | 285 | CB | LEU | 37 | 0.567 | 10.983 | 22.509 | 1.00 | 34.71 |
| ATOM | 286 | CG | LEU | 37 | 0.618 | 12.421 | 23.042 | 1.00 | 36.56 |
| ATOM | 287 | CD1 | LEU | 37 | 1.836 | 13.144 | 22.442 | 1.00 | 41.17 |
| ATOM | 288 | CD2 | LEU | 37 | −0.691 | 13.157 | 22.714 | 1.00 | 35.03 |
| ATOM | 289 | C | LEU | 37 | −0.374 | 8.682 | 22.475 | 1.00 | 27.21 |
| ATOM | 290 | O | LEU | 37 | −0.263 | 8.578 | 21.263 | 1.00 | 26.50 |
| ATOM | 291 | N | ASP | 38 | −0.392 | 7.632 | 23.287 | 1.00 | 28.84 |
| ATOM | 292 | CA | ASP | 38 | −0.250 | 6.280 | 22.763 | 1.00 | 33.05 |
| ATOM | 293 | CB | ASP | 38 | −0.363 | 5.233 | 23.889 | 1.00 | 34.31 |
| ATOM | 294 | CG | ASP | 38 | 0.851 | 5.229 | 24.810 | 1.00 | 36.92 |
| ATOM | 295 | OD1 | ASP | 38 | 0.986 | 4.326 | 25.669 | 1.00 | 39.75 |
| ATOM | 296 | OD2 | ASP | 38 | 1.681 | 6.140 | 24.679 | 1.00 | 40.38 |
| ATOM | 297 | C | ASP | 38 | −1.228 | 5.942 | 21.630 | 1.00 | 34.44 |
| ATOM | 298 | O | ASP | 38 | −0.852 | 5.245 | 20.694 | 1.00 | 34.98 |
| ATOM | 299 | N | GLY | 39 | −2.452 | 6.465 | 21.713 | 1.00 | 34.31 |
| ATOM | 300 | CA | GLY | 39 | −3.478 | 6.194 | 20.719 | 1.00 | 34.90 |
| ATOM | 301 | C | GLY | 39 | −3.412 | 6.898 | 19.370 | 1.00 | 35.63 |
| ATOM | 302 | O | GLY | 39 | −4.078 | 6.470 | 18.430 | 1.00 | 36.62 |
| ATOM | 303 | N | ILE | 40 | −2.634 | 7.967 | 19.253 | 1.00 | 35.51 |
| ATOM | 304 | CA | ILE | 40 | −2.510 | 8.667 | 17.972 | 1.00 | 35.87 |
| ATOM | 305 | CB | ILE | 40 | −1.503 | 9.862 | 18.082 | 1.00 | 36.81 |
| ATOM | 306 | CG2 | ILE | 40 | −1.254 | 10.491 | 16.712 | 1.00 | 33.51 |
| ATOM | 307 | CG1 | ILE | 40 | −2.061 | 10.913 | 19.047 | 1.00 | 37.34 |
| ATOM | 308 | CD1 | ILE | 40 | −1.192 | 12.161 | 19.151 | 1.00 | 36.05 |
| ATOM | 309 | C | ILE | 40 | −2.049 | 7.721 | 16.851 | 1.00 | 34.49 |
| ATOM | 310 | O | ILE | 40 | −1.033 | 7.048 | 16.979 | 1.00 | 32.65 |
| ATOM | 311 | N | VAL | 41 | −2.807 | 7.668 | 15.756 | 1.00 | 37.61 |
| ATOM | 312 | CA | VAL | 41 | −2.460 | 6.800 | 14.620 | 1.00 | 37.11 |
| ATOM | 313 | CB | VAL | 41 | −3.707 | 6.340 | 13.802 | 1.00 | 40.13 |
| ATOM | 314 | CG1 | VAL | 41 | −4.697 | 5.617 | 14.702 | 1.00 | 41.21 |
| ATOM | 315 | CG2 | VAL | 41 | −4.376 | 7.524 | 13.129 | 1.00 | 42.84 |
| ATOM | 316 | C | VAL | 41 | −1.522 | 7.521 | 13.669 | 1.00 | 35.61 |
| ATOM | 317 | O | VAL | 41 | −1.773 | 8.640 | 13.247 | 1.00 | 34.78 |
| ATOM | 318 | N | VAL | 42 | −0.430 | 6.867 | 13.330 | 1.00 | 37.16 |
| ATOM | 319 | CA | VAL | 42 | 0.546 | 7.462 | 12.444 | 1.00 | 39.92 |
| ATOM | 320 | CB | VAL | 42 | 1.926 | 7.532 | 13.136 | 1.00 | 37.79 |
| ATOM | 321 | CG1 | VAL | 42 | 2.927 | 8.217 | 12.232 | 1.00 | 37.61 |
| ATOM | 322 | CG2 | VAL | 42 | 1.809 | 8.259 | 14.460 | 1.00 | 37.43 |
| ATOM | 323 | C | VAL | 42 | 0.689 | 6.621 | 11.182 | 1.00 | 42.15 |
| ATOM | 324 | O | VAL | 42 | 0.644 | 5.395 | 11.245 | 1.00 | 40.81 |
| ATOM | 325 | N | GLU | 43 | 0.829 | 7.286 | 10.040 | 1.00 | 44.26 |
| ATOM | 326 | CA | GLU | 43 | 1.050 | 6.589 | 8.780 | 1.00 | 47.41 |
| ATOM | 327 | CB | GLU | 43 | 0.837 | 7.522 | 7.592 | 1.00 | 50.11 |
| ATOM | 328 | CG | GLU | 43 | −0.619 | 7.764 | 7.211 | 1.00 | 55.36 |
| ATOM | 329 | CD | GLU | 43 | −1.297 | 6.497 | 6.686 | 1.00 | 60.26 |
| ATOM | 330 | OE1 | GLU | 43 | −1.414 | 5.508 | 7.454 | 1.00 | 59.78 |
| ATOM | 331 | OE2 | GLU | 43 | −1.702 | 6.482 | 5.497 | 1.00 | 63.46 |
| ATOM | 332 | C | GLU | 43 | 2.496 | 6.121 | 8.800 | 1.00 | 48.82 |
| ATOM | 333 | O | GLU | 43 | 3.420 | 6.928 | 8.699 | 1.00 | 48.94 |
| ATOM | 334 | N | TYR | 44 | 2.691 | 4.817 | 8.943 | 1.00 | 50.69 |
| ATOM | 335 | CA | TYR | 44 | 4.032 | 4.237 | 8.999 | 1.00 | 52.56 |
| ATOM | 336 | CB | TYR | 44 | 4.193 | 3.521 | 10.337 | 1.00 | 51.42 |
| ATOM | 337 | CG | TYR | 44 | 5.560 | 2.957 | 10.626 | 1.00 | 51.48 |
| ATOM | 338 | CD1 | TYR | 44 | 6.670 | 3.791 | 10.773 | 1.00 | 49.46 |
| ATOM | 339 | CE1 | TYR | 44 | 7.919 | 3.268 | 11.107 | 1.00 | 49.00 |
| ATOM | 340 | CD2 | TYR | 44 | 5.739 | 1.581 | 10.811 | 1.00 | 50.45 |
| ATOM | 341 | CE2 | TYR | 44 | 6.982 | 1.055 | 11.143 | 1.00 | 49.22 |
| ATOM | 342 | CZ | TYR | 44 | 8.064 | 1.898 | 11.295 | 1.00 | 48.06 |
| ATOM | 343 | OH | TYR | 44 | 9.268 | 1.366 | 11.682 | 1.00 | 47.29 |
| ATOM | 344 | C | TYR | 44 | 4.165 | 3.263 | 7.832 | 1.00 | 54.71 |
| ATOM | 345 | O | TYR | 44 | 3.643 | 2.147 | 7.873 | 1.00 | 55.85 |
| ATOM | 346 | N | TYR | 45 | 4.864 | 3.708 | 6.790 | 1.00 | 58.86 |
| ATOM | 347 | CA | TYR | 45 | 5.067 | 2.941 | 5.552 | 1.00 | 60.22 |
| ATOM | 348 | CB | TYR | 45 | 6.152 | 1.879 | 5.721 | 1.00 | 60.58 |
| ATOM | 349 | CG | TYR | 45 | 7.497 | 2.434 | 6.123 | 1.00 | 60.52 |
| ATOM | 350 | CD1 | TYR | 45 | 7.701 | 2.944 | 7.400 | 1.00 | 60.80 |
| ATOM | 351 | CE1 | TYR | 45 | 8.937 | 3.464 | 7.780 | 1.00 | 61.27 |
| ATOM | 352 | CD2 | TYR | 45 | 8.564 | 2.458 | 5.226 | 1.00 | 59.56 |
| ATOM | 353 | CE2 | TYR | 45 | 9.801 | 2.977 | 5.596 | 1.00 | 61.01 |
| ATOM | 354 | CZ | TYR | 45 | 9.979 | 3.482 | 6.881 | 1.00 | 60.45 |
| ATOM | 355 | OH | TYR | 45 | 11.185 | 4.030 | 7.276 | 1.00 | 62.38 |
| ATOM | 356 | C | TYR | 45 | 3.782 | 2.280 | 5.076 | 1.00 | 61.45 |
| ATOM | 357 | O | TYR | 45 | 3.724 | 1.064 | 4.912 | 1.00 | 61.25 |
| ATOM | 358 | N | GLY | 46 | 2.751 | 3.100 | 4.873 | 1.00 | 64.05 |
| ATOM | 359 | CA | GLY | 46 | 1.461 | 2.608 | 4.407 | 1.00 | 65.41 |
| ATOM | 360 | C | GLY | 46 | 0.458 | 2.193 | 5.473 | 1.00 | 66.35 |
| ATOM | 361 | O | GLY | 46 | −0.737 | 2.509 | 5.368 | 1.00 | 66.56 |
| ATOM | 362 | N | THR | 47 | 0.952 | 1.501 | 6.501 | 1.00 | 66.38 |
| ATOM | 363 | CA | THR | 47 | 0.126 | 0.982 | 7.594 | 1.00 | 65.82 |
| ATOM | 364 | CB | THR | 47 | 0.711 | −0.347 | 8.135 | 1.00 | 67.46 |
| ATOM | 365 | OG1 | THR | 47 | 2.011 | −0.101 | 8.693 | 1.00 | 69.00 |
| ATOM | 366 | CG2 | THR | 47 | 0.842 | −1.375 | 7.012 | 1.00 | 67.57 |
| ATOM | 367 | C | THR | 47 | −0.058 | 1.920 | 8.789 | 1.00 | 63.78 |
| ATOM | 368 | O | THR | 47 | 0.912 | 2.391 | 9.393 | 1.00 | 63.80 |
| ATOM | 369 | N | PRO | 48 | −1.317 | 2.208 | 9.145 | 1.00 | 61.42 |
| ATOM | 370 | CD | PRO | 48 | −2.587 | 1.799 | 8.522 | 1.00 | 59.86 |
| ATOM | 371 | CA | PRO | 48 | −1.577 | 3.092 | 10.286 | 1.00 | 58.41 |
| ATOM | 372 | CB | PRO | 48 | −3.097 | 3.296 | 10.217 | 1.00 | 58.67 |
| ATOM | 373 | CG | PRO | 48 | −3.403 | 3.068 | 8.701 | 1.00 | 59.94 |
| ATOM | 374 | C | PRO | 48 | −1.137 | 2.360 | 11.550 | 1.00 | 54.30 |
| ATOM | 375 | O | PRO | 48 | −1.545 | 1.228 | 11.790 | 1.00 | 53.93 |
| ATOM | 376 | N | THR | 49 | −0.287 | 2.994 | 12.344 | 1.00 | 49.82 |
| ATOM | 377 | CA | THR | 49 | 0.191 | 2.363 | 13.560 | 1.00 | 45.54 |
| ATOM | 378 | CB | THR | 49 | 1.626 | 1.811 | 13.384 | 1.00 | 45.94 |
| ATOM | 379 | OG1 | THR | 49 | 2.548 | 2.895 | 13.261 | 1.00 | 51.01 |
| ATOM | 380 | CG2 | THR | 49 | 1.724 | 0.975 | 12.129 | 1.00 | 44.44 |
| ATOM | 381 | C | THR | 49 | 0.167 | 3.344 | 14.723 | 1.00 | 41.67 |
| ATOM | 382 | O | THR | 49 | 0.462 | 4.526 | 14.563 | 1.00 | 39.81 |
| ATOM | 383 | N | PRO | 50 | −0.207 | 2.861 | 15.915 | 1.00 | 38.96 |
| ATOM | 384 | CD | PRO | 50 | −0.608 | 1.492 | 16.275 | 1.00 | 36.47 |
| ATOM | 385 | CA | PRO | 50 | −0.258 | 3.731 | 17.087 | 1.00 | 35.39 |
| ATOM | 386 | CB | PRO | 50 | −0.768 | 2.787 | 18.189 | 1.00 | 35.03 |
| ATOM | 387 | CG | PRO | 50 | −1.589 | 1.770 | 17.391 | 1.00 | 37.74 |
| ATOM | 388 | C | PRO | 50 | 1.134 | 4.282 | 17.372 | 1.00 | 34.29 |
| ATOM | 389 | O | PRO | 50 | 2.132 | 3.566 | 17.222 | 1.00 | 32.88 |
| ATOM | 390 | N | LEU | 51 | 1.177 | 5.553 | 17.777 | 1.00 | 32.65 |
| ATOM | 391 | CA | LEU | 51 | 2.408 | 6.261 | 18.106 | 1.00 | 32.71 |
| ATOM | 392 | CB | LEU | 51 | 2.057 | 7.654 | 18.627 | 1.00 | 32.91 |
| ATOM | 393 | CG | LEU | 51 | 3.183 | 8.543 | 19.140 | 1.00 | 32.51 |
| ATOM | 394 | CD1 | LEU | 51 | 4.317 | 8.566 | 18.117 | 1.00 | 35.14 |
| ATOM | 395 | CD2 | LEU | 51 | 2.644 | 9.950 | 19.354 | 1.00 | 34.75 |
| ATOM | 396 | C | LEU | 51 | 3.259 | 5.527 | 19.146 | 1.00 | 35.19 |
| ATOM | 397 | O | LEU | 51 | 4.500 | 5.620 | 19.153 | 1.00 | 32.93 |
| ATOM | 398 | N | ARG | 52 | 2.573 | 4.817 | 20.036 | 1.00 | 35.13 |
| ATOM | 399 | CA | ARG | 52 | 3.205 | 4.055 | 21.102 | 1.00 | 35.65 |
| ATOM | 400 | CB | ARG | 52 | 2.122 | 3.263 | 21.853 | 1.00 | 35.45 |
| ATOM | 401 | CG | ARG | 52 | 2.629 | 2.303 | 22.934 | 1.00 | 37.41 |
| ATOM | 402 | CD | ARG | 52 | 1.456 | 1.582 | 23.582 | 1.00 | 39.06 |
| ATOM | 403 | NE | ARG | 52 | 0.576 | 1.021 | 22.547 | 1.00 | 44.41 |
| ATOM | 404 | CZ | ARG | 52 | 0.722 | −0.168 | 21.949 | 1.00 | 44.42 |
| ATOM | 405 | NH1 | ARG | 52 | 1.726 | −0.983 | 22.266 | 1.00 | 41.83 |
| ATOM | 406 | NH2 | ARG | 52 | −0.138 | −0.526 | 21.005 | 1.00 | 42.08 |
| ATOM | 407 | C | ARG | 52 | 4.299 | 3.103 | 20.586 | 1.00 | 34.33 |
| ATOM | 408 | O | ARG | 52 | 5.304 | 2.914 | 21.241 | 1.00 | 35.09 |
| ATOM | 409 | N | GLN | 53 | 4.092 | 2.519 | 19.417 | 1.00 | 34.91 |
| ATOM | 410 | CA | GLN | 53 | 5.049 | 1.572 | 18.837 | 1.00 | 38.97 |
| ATOM | 411 | CB | GLN | 53 | 4.322 | 0.539 | 17.959 | 1.00 | 40.17 |
| ATOM | 412 | CG | GLN | 53 | 3.235 | −0.225 | 18.663 | 1.00 | 46.58 |
| ATOM | 413 | CD | GLN | 53 | 2.466 | −1.151 | 17.736 | 1.00 | 50.36 |
| ATOM | 414 | OE1 | GLN | 53 | 1.897 | −0.727 | 16.727 | 1.00 | 50.89 |
| ATOM | 415 | NE2 | GLN | 53 | 2.429 | −2.431 | 18.090 | 1.00 | 55.52 |
| ATOM | 416 | C | GLN | 53 | 6.111 | 2.244 | 17.966 | 1.00 | 39.42 |
| ATOM | 417 | O | GLN | 53 | 6.975 | 1.577 | 17.447 | 1.00 | 36.91 |
| ATOM | 418 | N | LEU | 54 | 6.033 | 3.560 | 17.808 | 1.00 | 40.51 |
| ATOM | 419 | CA | LEU | 54 | 6.967 | 4.272 | 16.963 | 1.00 | 40.80 |
| ATOM | 420 | CB | LEU | 54 | 6.179 | 4.999 | 15.868 | 1.00 | 39.66 |
| ATOM | 421 | CG | LEU | 54 | 5.261 | 4.086 | 15.042 | 1.00 | 44.30 |
| ATOM | 422 | CD1 | LEU | 54 | 4.362 | 4.929 | 14.106 | 1.00 | 42.27 |
| ATOM | 423 | CD2 | LEU | 54 | 6.102 | 3.085 | 14.243 | 1.00 | 40.97 |
| ATOM | 424 | C | LEU | 54 | 7.823 | 5.269 | 17.739 | 1.00 | 40.78 |
| ATOM | 425 | O | LEU | 54 | 8.772 | 5.817 | 17.214 | 1.00 | 41.97 |
| ATOM | 426 | N | ALA | 55 | 7.506 | 5.510 | 18.998 | 1.00 | 40.71 |
| ATOM | 427 | CA | ALA | 55 | 8.285 | 6.485 | 19.734 | 1.00 | 40.64 |
| ATOM | 428 | CB | ALA | 55 | 7.855 | 7.889 | 19.332 | 1.00 | 38.83 |
| ATOM | 429 | C | ALA | 55 | 8.180 | 6.352 | 21.232 | 1.00 | 40.46 |
| ATOM | 430 | O | ALA | 55 | 7.256 | 5.747 | 21.759 | 1.00 | 40.59 |
| ATOM | 431 | N | SER | 56 | 9.166 | 6.927 | 21.898 | 1.00 | 41.32 |
| ATOM | 432 | CA | SER | 56 | 9.243 | 6.982 | 23.346 | 1.00 | 41.03 |
| ATOM | 433 | CB | SER | 56 | 10.715 | 6.932 | 23.771 | 1.00 | 44.77 |
| ATOM | 434 | OG | SER | 56 | 10.860 | 7.109 | 25.169 | 1.00 | 49.29 |
| ATOM | 435 | C | SER | 56 | 8.635 | 8.339 | 23.718 | 1.00 | 39.72 |
| ATOM | 436 | O | SER | 56 | 9.024 | 9.365 | 23.151 | 1.00 | 37.62 |
| ATOM | 437 | N | VAL | 57 | 7.667 | 8.350 | 24.634 | 1.00 | 40.18 |
| ATOM | 438 | CA | VAL | 57 | 7.041 | 9.605 | 25.059 | 1.00 | 38.81 |
| ATOM | 439 | CB | VAL | 57 | 5.503 | 9.601 | 24.817 | 1.00 | 38.08 |
| ATOM | 440 | CG1 | VAL | 57 | 4.885 | 10.935 | 25.240 | 1.00 | 35.66 |
| ATOM | 441 | CG2 | VAL | 57 | 5.218 | 9.368 | 23.360 | 1.00 | 34.50 |
| ATOM | 442 | C | VAL | 57 | 7.334 | 9.821 | 26.530 | 1.00 | 41.07 |
| ATOM | 443 | O | VAL | 57 | 6.827 | 9.096 | 27.386 | 1.00 | 43.17 |
| ATOM | 444 | N | THR | 58 | 8.159 | 10.828 | 26.814 | 1.00 | 42.76 |
| ATOM | 445 | CA | THR | 58 | 8.577 | 11.160 | 28.173 | 1.00 | 44.28 |
| ATOM | 446 | CB | THR | 58 | 10.109 | 11.316 | 28.251 | 1.00 | 44.97 |
| ATOM | 447 | OG1 | THR | 58 | 10.730 | 10.029 | 28.151 | 1.00 | 50.84 |
| ATOM | 448 | CG2 | THR | 58 | 10.522 | 11.948 | 29.563 | 1.00 | 50.51 |
| ATOM | 449 | C | THR | 58 | 7.953 | 12.444 | 28.670 | 1.00 | 46.01 |
| ATOM | 450 | O | THR | 58 | 7.716 | 13.373 | 27.888 | 1.00 | 47.72 |
| ATOM | 451 | N | VAL | 59 | 7.689 | 12.516 | 29.971 | 1.00 | 45.39 |
| ATOM | 452 | CA | VAL | 59 | 7.107 | 13.736 | 30.522 | 1.00 | 45.59 |
| ATOM | 453 | CB | VAL | 59 | 6.336 | 13.468 | 31.829 | 1.00 | 42.70 |
| ATOM | 454 | CG1 | VAL | 59 | 5.726 | 14.768 | 32.327 | 1.00 | 42.59 |
| ATOM | 455 | CG2 | VAL | 59 | 5.272 | 12.408 | 31.613 | 1.00 | 41.35 |
| ATOM | 456 | C | VAL | 59 | 8.218 | 14.753 | 30.809 | 1.00 | 46.24 |
| ATOM | 457 | O | VAL | 59 | 8.936 | 14.645 | 31.801 | 1.00 | 44.55 |
| ATOM | 458 | N | GLU | 60 | 8.369 | 15.736 | 29.933 | 1.00 | 48.24 |
| ATOM | 459 | CA | GLU | 60 | 9.400 | 16.740 | 30.136 | 1.00 | 50.27 |
| ATOM | 460 | CB | GLU | 60 | 9.559 | 17.588 | 28.868 | 1.00 | 50.29 |
| ATOM | 461 | CG | GLU | 60 | 10.513 | 18.765 | 28.996 | 1.00 | 53.37 |
| ATOM | 462 | CD | GLU | 60 | 10.795 | 19.442 | 27.659 | 1.00 | 53.53 |
| ATOM | 463 | OE1 | GLU | 60 | 11.657 | 18.958 | 26.912 | 1.00 | 56.23 |
| ATOM | 464 | OE2 | GLU | 60 | 10.145 | 20.453 | 27.336 | 1.00 | 55.58 |
| ATOM | 465 | C | GLU | 60 | 9.004 | 17.575 | 31.357 | 1.00 | 51.63 |
| ATOM | 466 | O | GLU | 60 | 9.825 | 17.863 | 32.216 | 1.00 | 51.46 |
| ATOM | 467 | N | ASP | 61 | 7.730 | 17.927 | 31.445 | 1.00 | 53.42 |
| ATOM | 468 | CA | ASP | 61 | 7.225 | 18.694 | 32.579 | 1.00 | 56.91 |
| ATOM | 469 | CB | ASP | 61 | 7.567 | 20.176 | 32.418 | 1.00 | 57.87 |
| ATOM | 470 | CG | ASP | 61 | 7.065 | 20.736 | 31.115 | 1.00 | 59.93 |
| ATOM | 471 | OD1 | ASP | 61 | 7.636 | 20.363 | 30.059 | 1.00 | 60.62 |
| ATOM | 472 | OD2 | ASP | 61 | 6.085 | 21.520 | 31.144 | 1.00 | 61.00 |
| ATOM | 473 | C | ASP | 61 | 5.701 | 18.531 | 32.694 | 1.00 | 57.24 |
| ATOM | 474 | O | ASP | 61 | 5.055 | 18.036 | 31.775 | 1.00 | 59.19 |
| ATOM | 475 | N | SER | 62 | 5.146 | 18.967 | 33.821 | 1.00 | 57.11 |
| ATOM | 476 | CA | SER | 62 | 3.714 | 18.893 | 34.120 | 1.00 | 56.21 |
| ATOM | 477 | CB | SER | 62 | 3.404 | 19.839 | 35.267 | 1.00 | 58.84 |
| ATOM | 478 | OG | SER | 62 | 3.730 | 21.172 | 34.891 | 1.00 | 61.16 |
| ATOM | 479 | C | SER | 62 | 2.727 | 19.186 | 32.980 | 1.00 | 54.83 |
| ATOM | 480 | O | SER | 62 | 1.606 | 18.675 | 32.988 | 1.00 | 54.24 |
| ATOM | 481 | N | ARG | 63 | 3.115 | 20.019 | 32.022 | 1.00 | 52.32 |
| ATOM | 482 | CA | ARG | 63 | 2.214 | 20.326 | 30.920 | 1.00 | 51.50 |
| ATOM | 483 | CB | ARG | 63 | 1.575 | 21.711 | 31.112 | 1.00 | 53.61 |
| ATOM | 484 | CG | ARG | 63 | 2.516 | 22.912 | 31.044 | 1.00 | 57.29 |
| ATOM | 485 | CD | ARG | 63 | 1.906 | 24.083 | 31.836 | 1.00 | 60.21 |
| ATOM | 486 | NE | ARG | 63 | 2.647 | 25.338 | 31.717 | 1.00 | 62.28 |
| ATOM | 487 | CZ | ARG | 63 | 2.565 | 26.160 | 30.675 | 1.00 | 65.53 |
| ATOM | 488 | NH1 | ARG | 63 | 1.767 | 25.860 | 29.651 | 1.00 | 66.70 |
| ATOM | 489 | NH2 | ARG | 63 | 3.272 | 27.289 | 30.657 | 1.00 | 65.73 |
| ATOM | 490 | C | ARG | 63 | 2.893 | 20.241 | 29.562 | 1.00 | 48.65 |
| ATOM | 491 | O | ARG | 63 | 2.458 | 20.872 | 28.606 | 1.00 | 49.55 |
| ATOM | 492 | N | THR | 64 | 3.946 | 19.438 | 29.473 | 1.00 | 44.74 |
| ATOM | 493 | CA | THR | 64 | 4.659 | 19.298 | 28.215 | 1.00 | 43.06 |
| ATOM | 494 | CB | THR | 64 | 5.827 | 20.305 | 28.134 | 1.00 | 42.31 |
| ATOM | 495 | OG1 | THR | 64 | 5.337 | 21.616 | 28.443 | 1.00 | 46.00 |
| ATOM | 496 | CG2 | THR | 64 | 6.440 | 20.321 | 26.726 | 1.00 | 41.82 |
| ATOM | 497 | C | THR | 64 | 5.215 | 17.884 | 27.983 | 1.00 | 41.37 |
| ATOM | 498 | O | THR | 64 | 5.806 | 17.270 | 28.888 | 1.00 | 38.27 |
| ATOM | 499 | N | LEU | 65 | 5.010 | 17.363 | 26.777 | 1.00 | 37.51 |
| ATOM | 500 | CA | LEU | 65 | 5.540 | 16.050 | 26.464 | 1.00 | 38.11 |
| ATOM | 501 | CB | LEU | 65 | 4.477 | 15.102 | 25.875 | 1.00 | 34.91 |
| ATOM | 502 | CG | LEU | 65 | 3.255 | 14.797 | 26.745 | 1.00 | 35.31 |
| ATOM | 503 | CD1 | LEU | 65 | 2.412 | 13.721 | 26.098 | 1.00 | 33.78 |
| ATOM | 504 | CD2 | LEU | 65 | 3.712 | 14.367 | 28.108 | 1.00 | 34.37 |
| ATOM | 505 | C | LEU | 65 | 6.669 | 16.170 | 25.473 | 1.00 | 38.28 |
| ATOM | 506 | O | LEU | 65 | 6.685 | 17.051 | 24.614 | 1.00 | 38.59 |
| ATOM | 507 | N | LYS | 66 | 7.615 | 15.254 | 25.613 | 1.00 | 40.15 |
| ATOM | 508 | CA | LYS | 66 | 8.774 | 15.161 | 24.743 | 1.00 | 40.02 |
| ATOM | 509 | CB | LYS | 66 | 10.043 | 15.189 | 25.588 | 1.00 | 45.80 |
| ATOM | 510 | CG | LYS | 66 | 11.202 | 14.454 | 24.977 | 1.00 | 51.87 |
| ATOM | 511 | CD | LYS | 66 | 12.272 | 14.216 | 26.026 | 1.00 | 56.23 |
| ATOM | 512 | CE | LYS | 66 | 13.346 | 13.286 | 25.476 | 1.00 | 59.78 |
| ATOM | 513 | NZ | LYS | 66 | 14.455 | 13.197 | 26.441 | 1.00 | 60.71 |
| ATOM | 514 | C | LYS | 66 | 8.609 | 13.823 | 24.031 | 1.00 | 37.57 |
| ATOM | 515 | O | LYS | 66 | 8.606 | 12.770 | 24.668 | 1.00 | 33.81 |
| ATOM | 516 | N | ILE | 67 | 8.474 | 13.871 | 22.715 | 1.00 | 36.18 |
| ATOM | 517 | CA | ILE | 67 | 8.313 | 12.666 | 21.923 | 1.00 | 37.12 |
| ATOM | 518 | CB | ILE | 67 | 7.241 | 12.841 | 20.853 | 1.00 | 36.65 |
| ATOM | 519 | CG2 | ILE | 67 | 7.057 | 11.546 | 20.108 | 1.00 | 39.63 |
| ATOM | 520 | CG1 | ILE | 67 | 5.911 | 13.234 | 21.473 | 1.00 | 34.46 |
| ATOM | 521 | CD1 | ILE | 67 | 4.873 | 13.466 | 20.423 | 1.00 | 35.05 |
| ATOM | 522 | C | ILE | 67 | 9.624 | 12.276 | 21.246 | 1.00 | 39.04 |
| ATOM | 523 | O | ILE | 67 | 10.195 | 13.035 | 20.466 | 1.00 | 39.66 |
| ATOM | 524 | N | ASN | 68 | 10.086 | 11.075 | 21.539 | 1.00 | 40.32 |
| ATOM | 525 | CA | ASN | 68 | 11.331 | 10.606 | 20.975 | 1.00 | 41.37 |
| ATOM | 526 | CB | ASN | 68 | 12.197 | 10.115 | 22.119 | 1.00 | 44.76 |
| ATOM | 527 | CG | ASN | 68 | 13.584 | 9.842 | 21.691 | 1.00 | 47.81 |
| ATOM | 528 | OD1 | ASN | 68 | 14.155 | 10.636 | 20.951 | 1.00 | 52.33 |
| ATOM | 529 | ND2 | ASN | 68 | 14.162 | 8.727 | 22.155 | 1.00 | 50.81 |
| ATOM | 530 | C | ASN | 68 | 11.107 | 9.511 | 19.920 | 1.00 | 40.11 |
| ATOM | 531 | O | ASN | 68 | 11.005 | 8.331 | 20.248 | 1.00 | 38.50 |
| ATOM | 532 | N | VAL | 69 | 11.031 | 9.922 | 18.657 | 1.00 | 38.66 |
| ATOM | 533 | CA | VAL | 69 | 10.794 | 9.008 | 17.539 | 1.00 | 39.78 |
| ATOM | 534 | CB | VAL | 69 | 10.383 | 9.806 | 16.277 | 1.00 | 38.68 |
| ATOM | 535 | CG1 | VAL | 69 | 9.997 | 8.867 | 15.165 | 1.00 | 39.74 |
| ATOM | 536 | CG2 | VAL | 69 | 9.221 | 10.733 | 16.600 | 1.00 | 37.97 |
| ATOM | 537 | C | VAL | 69 | 12.060 | 8.177 | 17.278 | 1.00 | 39.57 |
| ATOM | 538 | O | VAL | 69 | 13.156 | 8.741 | 17.193 | 1.00 | 40.97 |
| ATOM | 539 | N | PHE | 70 | 11.909 | 6.855 | 17.146 | 1.00 | 36.02 |
| ATOM | 540 | CA | PHE | 70 | 13.059 | 5.953 | 16.968 | 1.00 | 35.77 |
| ATOM | 541 | CB | PHE | 70 | 12.660 | 4.495 | 17.287 | 1.00 | 36.22 |
| ATOM | 542 | CG | PHE | 70 | 12.026 | 4.311 | 18.644 | 1.00 | 34.76 |
| ATOM | 543 | CD1 | PHE | 70 | 12.692 | 4.693 | 19.803 | 1.00 | 32.72 |
| ATOM | 544 | CD2 | PHE | 70 | 10.764 | 3.720 | 18.757 | 1.00 | 36.32 |
| ATOM | 545 | CE1 | PHE | 70 | 12.119 | 4.492 | 21.050 | 1.00 | 34.27 |
| ATOM | 546 | CE2 | PHE | 70 | 10.176 | 3.514 | 20.003 | 1.00 | 32.75 |
| ATOM | 547 | CZ | PHE | 70 | 10.857 | 3.901 | 21.157 | 1.00 | 34.27 |
| ATOM | 548 | C | PHE | 70 | 13.731 | 5.996 | 15.597 | 1.00 | 35.41 |
| ATOM | 549 | O | PHE | 70 | 14.947 | 5.811 | 15.474 | 1.00 | 35.74 |
| ATOM | 550 | N | ASP | 71 | 12.924 | 6.209 | 14.574 | 1.00 | 34.31 |
| ATOM | 551 | CA | ASP | 71 | 13.386 | 6.317 | 13.206 | 1.00 | 36.54 |
| ATOM | 552 | CB | ASP | 71 | 12.552 | 5.406 | 12.299 | 1.00 | 37.50 |
| ATOM | 553 | CG | ASP | 71 | 12.906 | 5.536 | 10.837 | 1.00 | 42.15 |
| ATOM | 554 | OD1 | ASP | 71 | 13.497 | 6.580 | 10.444 | 1.00 | 42.99 |
| ATOM | 555 | OD2 | ASP | 71 | 12.556 | 4.596 | 10.071 | 1.00 | 43.58 |
| ATOM | 556 | C | ASP | 71 | 13.156 | 7.797 | 12.855 | 1.00 | 36.95 |
| ATOM | 557 | O | ASP | 71 | 12.008 | 8.249 | 12.711 | 1.00 | 34.20 |
| ATOM | 558 | N | ARG | 72 | 14.261 | 8.530 | 12.743 | 1.00 | 37.72 |
| ATOM | 559 | CA | ARG | 72 | 14.260 | 9.957 | 12.453 | 1.00 | 41.91 |
| ATOM | 560 | CB | ARG | 72 | 15.714 | 10.444 | 12.340 | 1.00 | 44.20 |
| ATOM | 561 | CG | ARG | 72 | 15.897 | 11.931 | 12.101 | 1.00 | 50.61 |
| ATOM | 562 | CD | ARG | 72 | 17.379 | 12.304 | 12.148 | 1.00 | 54.03 |
| ATOM | 563 | NE | ARG | 72 | 17.935 | 12.179 | 13.495 | 1.00 | 58.08 |
| ATOM | 564 | CZ | ARG | 72 | 19.241 | 12.176 | 13.773 | 1.00 | 61.41 |
| ATOM | 565 | NH1 | ARG | 72 | 20.136 | 12.293 | 12.793 | 1.00 | 60.31 |
| ATOM | 566 | NH2 | ARG | 72 | 19.658 | 12.056 | 15.033 | 1.00 | 60.96 |
| ATOM | 567 | C | ARG | 72 | 13.447 | 10.317 | 11.205 | 1.00 | 42.06 |
| ATOM | 568 | O | ARG | 72 | 12.784 | 11.349 | 11.166 | 1.00 | 41.02 |
| ATOM | 569 | N | SER | 73 | 13.459 | 9.452 | 10.199 | 1.00 | 42.69 |
| ATOM | 570 | CA | SER | 73 | 12.700 | 9.719 | 8.991 | 1.00 | 41.82 |
| ATOM | 571 | CB | SER | 73 | 13.007 | 8.655 | 7.935 | 1.00 | 44.65 |
| ATOM | 572 | OG | SER | 73 | 12.665 | 7.354 | 8.392 | 1.00 | 47.00 |
| ATOM | 573 | C | SER | 73 | 11.190 | 9.779 | 9.233 | 1.00 | 42.72 |
| ATOM | 574 | O | SER | 73 | 10.449 | 10.336 | 8.417 | 1.00 | 42.14 |
| ATOM | 575 | N | MET | 74 | 10.730 | 9.199 | 10.338 | 1.00 | 41.95 |
| ATOM | 576 | CA | MET | 74 | 9.304 | 9.205 | 10.666 | 1.00 | 44.14 |
| ATOM | 577 | CB | MET | 74 | 8.932 | 7.959 | 11.479 | 1.00 | 44.72 |
| ATOM | 578 | CG | MET | 74 | 8.928 | 6.674 | 10.705 | 1.00 | 45.64 |
| ATOM | 579 | SD | MET | 74 | 7.787 | 6.778 | 9.304 | 1.00 | 49.27 |
| ATOM | 580 | CE | MET | 74 | 8.965 | 7.228 | 7.973 | 1.00 | 49.04 |
| ATOM | 581 | C | MET | 74 | 8.819 | 10.433 | 11.442 | 1.00 | 43.59 |
| ATOM | 582 | O | MET | 74 | 7.612 | 10.629 | 11.605 | 1.00 | 43.28 |
| ATOM | 583 | N | SER | 75 | 9.750 | 11.237 | 11.939 | 1.00 | 42.08 |
| ATOM | 584 | CA | SER | 75 | 9.381 | 12.406 | 12.715 | 1.00 | 42.19 |
| ATOM | 585 | CB | SER | 75 | 10.621 | 13.208 | 13.085 | 1.00 | 42.09 |
| ATOM | 586 | OG | SER | 75 | 10.281 | 14.280 | 13.941 | 1.00 | 46.20 |
| ATOM | 587 | C | SER | 75 | 8.376 | 13.301 | 11.991 | 1.00 | 42.07 |
| ATOM | 588 | O | SER | 75 | 7.364 | 13.698 | 12.574 | 1.00 | 40.55 |
| ATOM | 589 | N | PRO | 76 | 8.650 | 13.666 | 10.728 | 1.00 | 41.74 |
| ATOM | 590 | CD | PRO | 76 | 9.809 | 13.391 | 9.861 | 1.00 | 41.37 |
| ATOM | 591 | CA | PRO | 76 | 7.688 | 14.518 | 10.021 | 1.00 | 40.98 |
| ATOM | 592 | CB | PRO | 76 | 8.284 | 14.610 | 8.622 | 1.00 | 40.75 |
| ATOM | 593 | CG | PRO | 76 | 9.773 | 14.597 | 8.928 | 1.00 | 41.52 |
| ATOM | 594 | C | PRO | 76 | 6.283 | 13.904 | 10.027 | 1.00 | 40.31 |
| ATOM | 595 | O | PRO | 76 | 5.293 | 14.595 | 10.298 | 1.00 | 40.54 |
| ATOM | 596 | N | ALA | 77 | 6.207 | 12.607 | 9.737 | 1.00 | 38.47 |
| ATOM | 597 | CA | ALA | 77 | 4.926 | 11.897 | 9.711 | 1.00 | 39.23 |
| ATOM | 598 | CB | ALA | 77 | 5.130 | 10.451 | 9.209 | 1.00 | 39.21 |
| ATOM | 599 | C | ALA | 77 | 4.260 | 11.887 | 11.097 | 1.00 | 37.86 |
| ATOM | 600 | O | ALA | 77 | 3.042 | 11.977 | 11.204 | 1.00 | 37.27 |
| ATOM | 601 | N | VAL | 78 | 5.067 | 11.769 | 12.148 | 1.00 | 35.44 |
| ATOM | 602 | CA | VAL | 78 | 4.542 | 11.754 | 13.495 | 1.00 | 34.88 |
| ATOM | 603 | CB | VAL | 78 | 5.647 | 11.436 | 14.541 | 1.00 | 33.08 |
| ATOM | 604 | CG1 | VAL | 78 | 5.135 | 11.747 | 15.952 | 1.00 | 27.26 |
| ATOM | 605 | CG2 | VAL | 78 | 6.021 | 9.967 | 14.467 | 1.00 | 34.28 |
| ATOM | 606 | C | VAL | 78 | 3.942 | 13.107 | 13.812 | 1.00 | 36.71 |
| ATOM | 607 | O | VAL | 78 | 2.886 | 13.205 | 14.438 | 1.00 | 35.21 |
| ATOM | 608 | N | GLU | 79 | 4.641 | 14.153 | 13.388 | 1.00 | 38.95 |
| ATOM | 609 | CA | GLU | 79 | 4.193 | 15.507 | 13.628 | 1.00 | 41.23 |
| ATOM | 610 | CB | GLU | 79 | 5.248 | 16.517 | 13.183 | 1.00 | 42.75 |
| ATOM | 611 | CG | GLU | 79 | 4.774 | 17.950 | 13.309 | 1.00 | 49.69 |
| ATOM | 612 | CD | GLU | 79 | 5.928 | 18.925 | 13.461 | 1.00 | 53.09 |
| ATOM | 613 | OE1 | GLU | 79 | 6.899 | 18.828 | 12.664 | 1.00 | 56.30 |
| ATOM | 614 | OE2 | GLU | 79 | 5.858 | 19.784 | 14.370 | 1.00 | 50.54 |
| ATOM | 615 | C | GLU | 79 | 2.874 | 15.764 | 12.937 | 1.00 | 40.64 |
| ATOM | 616 | O | GLU | 79 | 1.996 | 16.380 | 13.517 | 1.00 | 43.12 |
| ATOM | 617 | N | LYS | 80 | 2.723 | 15.275 | 11.714 | 1.00 | 41.68 |
| ATOM | 618 | CA | LYS | 80 | 1.465 | 15.442 | 11.002 | 1.00 | 43.65 |
| ATOM | 619 | CB | LYS | 80 | 1.586 | 14.991 | 9.542 | 1.00 | 47.34 |
| ATOM | 620 | CG | LYS | 80 | 2.415 | 15.924 | 8.634 | 1.00 | 51.49 |
| ATOM | 621 | CD | LYS | 80 | 1.969 | 17.397 | 8.749 | 1.00 | 54.11 |
| ATOM | 622 | CE | LYS | 80 | 2.242 | 18.208 | 7.450 | 1.00 | 58.81 |
| ATOM | 623 | NZ | LYS | 80 | 3.659 | 18.188 | 6.930 | 1.00 | 59.99 |
| ATOM | 624 | C | LYS | 80 | 0.356 | 14.636 | 11.674 | 1.00 | 43.12 |
| ATOM | 625 | O | LYS | 80 | −0.800 | 15.077 | 11.748 | 1.00 | 43.15 |
| ATOM | 626 | N | ALA | 81 | 0.708 | 13.451 | 12.158 | 1.00 | 40.38 |
| ATOM | 627 | CA | ALA | 81 | −0.257 | 12.598 | 12.824 | 1.00 | 38.88 |
| ATOM | 628 | CB | ALA | 81 | 0.387 | 11.255 | 13.201 | 1.00 | 36.34 |
| ATOM | 629 | C | ALA | 81 | −0.785 | 13.302 | 14.072 | 1.00 | 39.56 |
| ATOM | 630 | O | ALA | 81 | −1.964 | 13.183 | 14.404 | 1.00 | 41.23 |
| ATOM | 631 | N | ILE | 82 | 0.074 | 14.045 | 14.766 | 1.00 | 39.16 |
| ATOM | 632 | CA | ILE | 82 | −0.380 | 14.720 | 15.971 | 1.00 | 38.15 |
| ATOM | 633 | CB | ILE | 82 | 0.780 | 15.267 | 16.803 | 1.00 | 35.54 |
| ATOM | 634 | CG2 | ILE | 82 | 0.241 | 16.060 | 17.965 | 1.00 | 33.27 |
| ATOM | 635 | CG1 | ILE | 82 | 1.626 | 14.111 | 17.351 | 1.00 | 35.88 |
| ATOM | 636 | CD1 | ILE | 82 | 2.878 | 14.562 | 18.122 | 1.00 | 36.02 |
| ATOM | 637 | C | ILE | 82 | −1.326 | 15.860 | 15.620 | 1.00 | 40.83 |
| ATOM | 638 | O | ILE | 82 | −2.373 | 16.019 | 16.231 | 1.00 | 39.39 |
| ATOM | 639 | N | MET | 83 | −0.965 | 16.660 | 14.633 | 1.00 | 42.37 |
| ATOM | 640 | CA | MET | 83 | −1.856 | 17.733 | 14.268 | 1.00 | 44.18 |
| ATOM | 641 | CB | MET | 83 | −1.238 | 18.589 | 13.164 | 1.00 | 47.58 |
| ATOM | 642 | CG | MET | 83 | 0.119 | 19.148 | 13.565 | 1.00 | 52.70 |
| ATOM | 643 | SD | MET | 83 | 0.871 | 20.375 | 12.470 | 1.00 | 58.98 |
| ATOM | 644 | CE | MET | 83 | 0.820 | 19.513 | 10.881 | 1.00 | 57.30 |
| ATOM | 645 | C | MET | 83 | −3.169 | 17.128 | 13.806 | 1.00 | 43.27 |
| ATOM | 646 | O | MET | 83 | −4.237 | 17.593 | 14.206 | 1.00 | 44.19 |
| ATOM | 647 | N | ALA | 84 | −3.081 | 16.044 | 13.034 | 1.00 | 41.67 |
| ATOM | 648 | CA | ALA | 84 | −4.260 | 15.376 | 12.456 | 1.00 | 40.91 |
| ATOM | 649 | CB | ALA | 84 | −3.821 | 14.343 | 11.427 | 1.00 | 39.91 |
| ATOM | 650 | C | ALA | 84 | −5.264 | 14.737 | 13.404 | 1.00 | 40.62 |
| ATOM | 651 | O | ALA | 84 | −6.427 | 14.590 | 13.059 | 1.00 | 40.16 |
| ATOM | 652 | N | SER | 85 | −4.823 | 14.338 | 14.586 | 1.00 | 40.70 |
| ATOM | 653 | CA | SER | 85 | −5.729 | 13.716 | 15.542 | 1.00 | 39.44 |
| ATOM | 654 | CB | SER | 85 | −4.956 | 13.233 | 16.774 | 1.00 | 37.78 |
| ATOM | 655 | OG | SER | 85 | −4.436 | 14.330 | 17.503 | 1.00 | 36.38 |
| ATOM | 656 | C | SER | 85 | −6.797 | 14.713 | 15.976 | 1.00 | 38.77 |
| ATOM | 657 | O | SER | 85 | −7.799 | 14.333 | 16.543 | 1.00 | 38.69 |
| ATOM | 658 | N | ASP | 86 | −6.550 | 15.993 | 15.724 | 1.00 | 40.30 |
| ATOM | 659 | CA | ASP | 86 | −7.477 | 17.073 | 16.070 | 1.00 | 39.72 |
| ATOM | 660 | CB | ASP | 86 | −8.750 | 16.964 | 15.244 | 1.00 | 40.60 |
| ATOM | 661 | CG | ASP | 86 | −9.690 | 18.137 | 15.462 | 1.00 | 43.77 |
| ATOM | 662 | OD1 | ASP | 86 | −9.259 | 19.302 | 15.305 | 1.00 | 45.04 |
| ATOM | 663 | OD2 | ASP | 86 | −10.872 | 17.896 | 15.782 | 1.00 | 45.78 |
| ATOM | 664 | C | ASP | 86 | −7.816 | 17.123 | 17.548 | 1.00 | 40.91 |
| ATOM | 665 | O | ASP | 86 | −8.980 | 17.131 | 17.948 | 1.00 | 40.62 |
| ATOM | 666 | N | LEU | 87 | −6.768 | 17.161 | 18.355 | 1.00 | 41.54 |
| ATOM | 667 | CA | LEU | 87 | −6.892 | 17.230 | 19.796 | 1.00 | 44.40 |
| ATOM | 668 | CB | LEU | 87 | −6.028 | 16.146 | 20.446 | 1.00 | 44.35 |
| ATOM | 669 | CG | LEU | 87 | −6.395 | 14.711 | 20.057 | 1.00 | 47.40 |
| ATOM | 670 | CD1 | LEU | 87 | −5.385 | 13.701 | 20.596 | 1.00 | 45.31 |
| ATOM | 671 | CD2 | LEU | 87 | −7.791 | 14.427 | 20.595 | 1.00 | 46.71 |
| ATOM | 672 | C | LEU | 87 | −6.452 | 18.614 | 20.273 | 1.00 | 46.06 |
| ATOM | 673 | O | LEU | 87 | −6.292 | 18.840 | 21.468 | 1.00 | 46.97 |
| ATOM | 674 | N | GLY | 88 | −6.245 | 19.527 | 19.324 | 1.00 | 48.12 |
| ATOM | 675 | CA | GLY | 88 | −5.841 | 20.883 | 19.654 | 1.00 | 47.94 |
| ATOM | 676 | C | GLY | 88 | −4.377 | 20.975 | 20.005 | 1.00 | 47.52 |
| ATOM | 677 | O | GLY | 88 | −3.891 | 22.030 | 20.404 | 1.00 | 47.24 |
| ATOM | 678 | N | LEU | 89 | −3.676 | 19.858 | 19.853 | 1.00 | 48.46 |
| ATOM | 679 | CA | LEU | 89 | −2.252 | 19.788 | 20.159 | 1.00 | 48.30 |
| ATOM | 680 | CB | LEU | 89 | −1.822 | 18.334 | 20.374 | 1.00 | 47.12 |
| ATOM | 681 | CG | LEU | 89 | −2.643 | 17.495 | 21.352 | 1.00 | 48.13 |
| ATOM | 682 | CD1 | LEU | 89 | −2.063 | 16.072 | 21.451 | 1.00 | 45.92 |
| ATOM | 683 | CD2 | LEU | 89 | −2.650 | 18.170 | 22.701 | 1.00 | 49.76 |
| ATOM | 684 | C | LEU | 89 | −1.400 | 20.404 | 19.043 | 1.00 | 49.93 |
| ATOM | 685 | O | LEU | 89 | −1.463 | 19.976 | 17.877 | 1.00 | 49.90 |
| ATOM | 686 | N | ASN | 90 | −0.611 | 21.410 | 19.423 | 1.00 | 49.86 |
| ATOM | 687 | CA | ASN | 90 | 0.292 | 22.120 | 18.523 | 1.00 | 50.18 |
| ATOM | 688 | CB | ASN | 90 | 0.305 | 23.625 | 18.852 | 1.00 | 51.76 |
| ATOM | 689 | CG | ASN | 90 | −1.056 | 24.282 | 18.689 | 1.00 | 51.96 |
| ATOM | 690 | OD1 | ASN | 90 | −1.630 | 24.289 | 17.604 | 1.00 | 52.89 |
| ATOM | 691 | ND2 | ASN | 90 | −1.576 | 24.845 | 19.774 | 1.00 | 55.10 |
| ATOM | 692 | C | ASN | 90 | 1.693 | 21.560 | 18.747 | 1.00 | 49.01 |
| ATOM | 693 | O | ASN | 90 | 2.419 | 22.030 | 19.617 | 1.00 | 47.33 |
| ATOM | 694 | N | PRO | 91 | 2.080 | 20.534 | 17.981 | 1.00 | 49.04 |
| ATOM | 695 | CD | PRO | 91 | 1.310 | 19.836 | 16.941 | 1.00 | 47.70 |
| ATOM | 696 | CA | PRO | 91 | 3.405 | 19.921 | 18.109 | 1.00 | 50.17 |
| ATOM | 697 | CB | PRO | 91 | 3.313 | 18.750 | 17.140 | 1.00 | 49.46 |
| ATOM | 698 | CG | PRO | 91 | 2.407 | 19.315 | 16.064 | 1.00 | 47.73 |
| ATOM | 699 | C | PRO | 91 | 4.519 | 20.901 | 17.734 | 1.00 | 52.02 |
| ATOM | 700 | O | PRO | 91 | 4.437 | 21.584 | 16.716 | 1.00 | 49.73 |
| ATOM | 701 | N | ASN | 92 | 5.551 | 20.978 | 18.569 | 1.00 | 55.38 |
| ATOM | 702 | CA | ASN | 92 | 6.677 | 21.876 | 18.316 | 1.00 | 56.64 |
| ATOM | 703 | CB | ASN | 92 | 6.907 | 22.779 | 19.527 | 1.00 | 57.88 |
| ATOM | 704 | CG | ASN | 92 | 5.868 | 23.895 | 19.624 | 1.00 | 61.72 |
| ATOM | 705 | OD1 | ASN | 92 | 5.687 | 24.507 | 20.679 | 1.00 | 63.08 |
| ATOM | 706 | ND2 | ASN | 92 | 5.195 | 24.178 | 18.509 | 1.00 | 61.74 |
| ATOM | 707 | C | ASN | 92 | 7.901 | 21.053 | 17.999 | 1.00 | 56.71 |
| ATOM | 708 | O | ASN | 92 | 8.540 | 20.511 | 18.889 | 1.00 | 56.47 |
| ATOM | 709 | N | SER | 93 | 8.212 | 20.957 | 16.711 | 1.00 | 58.68 |
| ATOM | 710 | CA | SER | 93 | 9.340 | 20.163 | 16.241 | 1.00 | 61.01 |
| ATOM | 711 | CB | SER | 93 | 9.026 | 19.614 | 14.846 | 1.00 | 60.28 |
| ATOM | 712 | OG | SER | 93 | 10.014 | 18.692 | 14.424 | 1.00 | 60.68 |
| ATOM | 713 | C | SER | 93 | 10.661 | 20.935 | 16.225 | 1.00 | 62.49 |
| ATOM | 714 | O | SER | 93 | 10.719 | 22.094 | 15.811 | 1.00 | 63.13 |
| ATOM | 715 | N | ALA | 94 | 11.724 | 20.281 | 16.677 | 1.00 | 63.76 |
| ATOM | 716 | CA | ALA | 94 | 13.019 | 20.930 | 16.716 | 1.00 | 65.86 |
| ATOM | 717 | CB | ALA | 94 | 12.971 | 22.123 | 17.669 | 1.00 | 65.68 |
| ATOM | 718 | C | ALA | 94 | 14.139 | 19.988 | 17.127 | 1.00 | 67.27 |
| ATOM | 719 | O | ALA | 94 | 14.181 | 19.508 | 18.260 | 1.00 | 67.78 |
| ATOM | 720 | N | GLY | 95 | 15.048 | 19.727 | 16.198 | 1.00 | 68.46 |
| ATOM | 721 | CA | GLY | 95 | 16.180 | 18.872 | 16.502 | 1.00 | 69.61 |
| ATOM | 722 | C | GLY | 95 | 15.883 | 17.489 | 17.045 | 1.00 | 69.64 |
| ATOM | 723 | O | GLY | 95 | 15.826 | 17.281 | 18.257 | 1.00 | 70.06 |
| ATOM | 724 | N | SER | 96 | 15.692 | 16.542 | 16.134 | 1.00 | 68.83 |
| ATOM | 725 | CA | SER | 96 | 15.434 | 15.151 | 16.487 | 1.00 | 68.81 |
| ATOM | 726 | CB | SER | 96 | 16.734 | 14.514 | 16.984 | 1.00 | 67.99 |
| ATOM | 727 | OG | SER | 96 | 17.708 | 14.490 | 15.954 | 1.00 | 69.91 |
| ATOM | 728 | C | SER | 96 | 14.320 | 14.901 | 17.514 | 1.00 | 68.02 |
| ATOM | 729 | O | SER | 96 | 13.902 | 13.759 | 17.726 | 1.00 | 69.37 |
| ATOM | 730 | N | ASP | 97 | 13.819 | 15.959 | 18.135 | 1.00 | 65.66 |
| ATOM | 731 | CA | ASP | 97 | 12.792 | 15.801 | 19.149 | 1.00 | 61.46 |
| ATOM | 732 | CB | ASP | 97 | 13.427 | 16.128 | 20.508 | 1.00 | 64.43 |
| ATOM | 733 | CG | ASP | 97 | 12.454 | 16.730 | 21.498 | 1.00 | 68.00 |
| ATOM | 734 | OD1 | ASP | 97 | 11.686 | 17.632 | 21.096 | 1.00 | 70.75 |
| ATOM | 735 | OD2 | ASP | 97 | 12.480 | 16.339 | 22.690 | 1.00 | 69.61 |
| ATOM | 736 | C | ASP | 97 | 11.563 | 16.657 | 18.874 | 1.00 | 57.75 |
| ATOM | 737 | O | ASP | 97 | 11.662 | 17.760 | 18.342 | 1.00 | 56.47 |
| ATOM | 738 | N | ILE | 98 | 10.399 | 16.116 | 19.220 | 1.00 | 54.15 |
| ATOM | 739 | CA | ILE | 98 | 9.129 | 16.810 | 19.063 | 1.00 | 48.95 |
| ATOM | 740 | CB | ILE | 98 | 8.069 | 15.928 | 18.372 | 1.00 | 48.45 |
| ATOM | 741 | CG2 | ILE | 98 | 6.724 | 16.639 | 18.364 | 1.00 | 46.97 |
| ATOM | 742 | CG1 | ILE | 98 | 8.519 | 15.571 | 16.955 | 1.00 | 47.46 |
| ATOM | 743 | CD1 | ILE | 98 | 7.566 | 14.651 | 16.229 | 1.00 | 45.26 |
| ATOM | 744 | C | ILE | 98 | 8.633 | 17.073 | 20.469 | 1.00 | 48.06 |
| ATOM | 745 | O | ILE | 98 | 8.669 | 16.187 | 21.310 | 1.00 | 47.48 |
| ATOM | 746 | N | ARG | 99 | 8.202 | 18.291 | 20.752 | 1.00 | 46.99 |
| ATOM | 747 | CA | ARG | 99 | 7.660 | 18.556 | 22.069 | 1.00 | 47.18 |
| ATOM | 748 | CB | ARG | 99 | 8.347 | 19.738 | 22.730 | 1.00 | 48.61 |
| ATOM | 749 | CG | ARG | 99 | 9.780 | 19.436 | 23.088 | 1.00 | 52.92 |
| ATOM | 750 | CD | ARG | 99 | 10.435 | 20.597 | 23.774 | 1.00 | 53.16 |
| ATOM | 751 | NE | ARG | 99 | 11.875 | 20.550 | 23.580 | 1.00 | 59.38 |
| ATOM | 752 | CZ | ARG | 99 | 12.656 | 19.533 | 23.930 | 1.00 | 62.25 |
| ATOM | 753 | NH1 | ARG | 99 | 12.137 | 18.453 | 24.507 | 1.00 | 63.64 |
| ATOM | 754 | NH2 | ARG | 99 | 13.963 | 19.594 | 23.687 | 1.00 | 64.01 |
| ATOM | 755 | C | ARG | 99 | 6.182 | 18.816 | 21.892 | 1.00 | 45.57 |
| ATOM | 756 | O | ARG | 99 | 5.748 | 19.325 | 20.853 | 1.00 | 45.31 |
| ATOM | 757 | N | VAL | 100 | 5.408 | 18.451 | 22.901 | 1.00 | 43.60 |
| ATOM | 758 | CA | VAL | 100 | 3.967 | 18.601 | 22.834 | 1.00 | 42.41 |
| ATOM | 759 | CB | VAL | 100 | 3.295 | 17.210 | 22.698 | 1.00 | 42.94 |
| ATOM | 760 | CG1 | VAL | 100 | 1.774 | 17.350 | 22.702 | 1.00 | 44.36 |
| ATOM | 761 | CG2 | VAL | 100 | 3.792 | 16.508 | 21.427 | 1.00 | 41.91 |
| ATOM | 762 | C | VAL | 100 | 3.417 | 19.310 | 24.065 | 1.00 | 41.58 |
| ATOM | 763 | O | VAL | 100 | 3.209 | 18.698 | 25.110 | 1.00 | 40.47 |
| ATOM | 764 | N | PRO | 101 | 3.236 | 20.632 | 23.977 | 1.00 | 41.65 |
| ATOM | 765 | CD | PRO | 101 | 3.568 | 21.570 | 22.889 | 1.00 | 41.07 |
| ATOM | 766 | CA | PRO | 101 | 2.696 | 21.361 | 25.122 | 1.00 | 39.69 |
| ATOM | 767 | CB | PRO | 101 | 2.830 | 22.809 | 24.675 | 1.00 | 40.75 |
| ATOM | 768 | CG | PRO | 101 | 2.608 | 22.690 | 23.169 | 1.00 | 40.97 |
| ATOM | 769 | C | PRO | 101 | 1.243 | 20.906 | 25.263 | 1.00 | 38.36 |
| ATOM | 770 | O | PRO | 101 | 0.566 | 20.638 | 24.273 | 1.00 | 36.38 |
| ATOM | 771 | N | LEU | 102 | 0.766 | 20.816 | 26.491 | 1.00 | 37.40 |
| ATOM | 772 | CA | LEU | 102 | −0.597 | 20.349 | 26.728 | 1.00 | 36.57 |
| ATOM | 773 | CB | LEU | 102 | −0.536 | 19.081 | 27.605 | 1.00 | 36.65 |
| ATOM | 774 | CG | LEU | 102 | 0.247 | 17.915 | 26.968 | 1.00 | 34.92 |
| ATOM | 775 | CD1 | LEU | 102 | 0.509 | 16.837 | 27.969 | 1.00 | 34.58 |
| ATOM | 776 | CD2 | LEU | 102 | −0.518 | 17.391 | 25.785 | 1.00 | 31.23 |
| ATOM | 777 | C | LEU | 102 | −1.536 | 21.375 | 27.364 | 1.00 | 35.46 |
| ATOM | 778 | O | LEU | 102 | −1.148 | 22.129 | 28.247 | 1.00 | 32.96 |
| ATOM | 779 | N | PRO | 103 | −2.809 | 21.379 | 26.945 | 1.00 | 37.48 |
| ATOM | 780 | CD | PRO | 103 | −3.456 | 20.510 | 25.948 | 1.00 | 38.55 |
| ATOM | 781 | CA | PRO | 103 | −3.797 | 22.311 | 27.485 | 1.00 | 39.19 |
| ATOM | 782 | CB | PRO | 103 | −5.015 | 22.043 | 26.599 | 1.00 | 38.49 |
| ATOM | 783 | CG | PRO | 103 | −4.921 | 20.592 | 26.384 | 1.00 | 36.60 |
| ATOM | 784 | C | PRO | 103 | −4.055 | 21.999 | 28.960 | 1.00 | 41.74 |
| ATOM | 785 | O | PRO | 103 | −3.955 | 20.850 | 29.388 | 1.00 | 41.12 |
| ATOM | 786 | N | PRO | 104 | −4.386 | 23.018 | 29.763 | 1.00 | 45.20 |
| ATOM | 787 | CD | PRO | 104 | −4.533 | 24.458 | 29.473 | 1.00 | 46.97 |
| ATOM | 788 | CA | PRO | 104 | −4.643 | 22.772 | 31.184 | 1.00 | 44.68 |
| ATOM | 789 | CB | PRO | 104 | −4.783 | 24.177 | 31.745 | 1.00 | 44.21 |
| ATOM | 790 | CG | PRO | 104 | −5.461 | 24.893 | 30.601 | 1.00 | 47.15 |
| ATOM | 791 | C | PRO | 104 | −5.911 | 21.957 | 31.390 | 1.00 | 45.36 |
| ATOM | 792 | O | PRO | 104 | −6.735 | 21.812 | 30.480 | 1.00 | 46.09 |
| ATOM | 793 | N | LEU | 105 | −6.056 | 21.400 | 32.580 | 1.00 | 44.16 |
| ATOM | 794 | CA | LEU | 105 | −7.260 | 20.654 | 32.889 | 1.00 | 43.98 |
| ATOM | 795 | CB | LEU | 105 | −7.208 | 20.155 | 34.327 | 1.00 | 42.52 |
| ATOM | 796 | CG | LEU | 105 | −6.071 | 19.208 | 34.668 | 1.00 | 41.22 |
| ATOM | 797 | CD1 | LEU | 105 | −6.052 | 18.923 | 36.174 | 1.00 | 40.33 |
| ATOM | 798 | CD2 | LEU | 105 | −6.263 | 17.944 | 33.861 | 1.00 | 39.80 |
| ATOM | 799 | C | LEU | 105 | −8.398 | 21.655 | 32.739 | 1.00 | 43.38 |
| ATOM | 800 | O | LEU | 105 | −8.199 | 22.860 | 32.924 | 1.00 | 44.06 |
| ATOM | 801 | N | THR | 106 | −9.588 | 21.175 | 32.412 | 1.00 | 41.61 |
| ATOM | 802 | CA | THR | 106 | −10.709 | 22.087 | 32.255 | 1.00 | 42.62 |
| ATOM | 803 | CB | THR | 106 | −11.943 | 21.367 | 31.673 | 1.00 | 40.92 |
| ATOM | 804 | OG1 | THR | 106 | −12.315 | 20.274 | 32.518 | 1.00 | 38.13 |
| ATOM | 805 | CG2 | THR | 106 | −11.626 | 20.847 | 30.278 | 1.00 | 41.50 |
| ATOM | 806 | C | THR | 106 | −11.095 | 22.774 | 33.566 | 1.00 | 43.42 |
| ATOM | 807 | O | THR | 106 | −10.777 | 22.301 | 34.651 | 1.00 | 40.88 |
| ATOM | 808 | N | GLU | 107 | −11.777 | 23.903 | 33.439 | 1.00 | 46.29 |
| ATOM | 809 | CA | GLU | 107 | −12.225 | 24.682 | 34.578 | 1.00 | 50.04 |
| ATOM | 810 | CB | GLU | 107 | −13.100 | 25.840 | 34.094 | 1.00 | 55.08 |
| ATOM | 811 | CG | GLU | 107 | −12.939 | 27.144 | 34.864 | 1.00 | 61.74 |
| ATOM | 812 | CD | GLU | 107 | −11.783 | 27.997 | 34.337 | 1.00 | 65.24 |
| ATOM | 813 | OE1 | GLU | 107 | −11.844 | 28.413 | 33.158 | 1.00 | 66.53 |
| ATOM | 814 | OE2 | GLU | 107 | −10.820 | 28.257 | 35.096 | 1.00 | 68.36 |
| ATOM | 815 | C | GLU | 107 | −13.064 | 23.760 | 35.464 | 1.00 | 49.91 |
| ATOM | 816 | O | GLU | 107 | −12.860 | 23.673 | 36.677 | 1.00 | 49.39 |
| ATOM | 817 | N | GLU | 108 | −14.004 | 23.067 | 34.835 | 1.00 | 49.83 |
| ATOM | 818 | CA | GLU | 108 | −14.894 | 22.146 | 35.526 | 1.00 | 50.79 |
| ATOM | 819 | CB | GLU | 108 | −15.887 | 21.532 | 34.523 | 1.00 | 52.77 |
| ATOM | 820 | CG | GLU | 108 | −17.030 | 20.740 | 35.160 | 1.00 | 57.30 |
| ATOM | 821 | CD | GLU | 108 | −17.865 | 21.574 | 36.143 | 1.00 | 60.57 |
| ATOM | 822 | OE1 | GLU | 108 | −18.370 | 22.655 | 35.757 | 1.00 | 61.21 |
| ATOM | 823 | OE2 | GLU | 108 | −18.018 | 21.141 | 37.310 | 1.00 | 63.46 |
| ATOM | 824 | C | GLU | 108 | −14.126 | 21.043 | 36.262 | 1.00 | 49.48 |
| ATOM | 825 | O | GLU | 108 | −14.509 | 20.636 | 37.369 | 1.00 | 48.71 |
| ATOM | 826 | N | ARG | 109 | −13.042 | 20.566 | 35.656 | 1.00 | 47.01 |
| ATOM | 827 | CA | ARG | 109 | −12.224 | 19.528 | 36.278 | 1.00 | 45.40 |
| ATOM | 828 | CB | ARG | 109 | −11.211 | 18.982 | 35.270 | 1.00 | 45.36 |
| ATOM | 829 | CG | ARG | 109 | −10.308 | 17.876 | 35.792 | 1.00 | 46.17 |
| ATOM | 830 | CD | ARG | 109 | −11.096 | 16.681 | 36.378 | 1.00 | 48.74 |
| ATOM | 831 | NE | ARG | 109 | −10.190 | 15.611 | 36.810 | 1.00 | 51.07 |
| ATOM | 832 | CZ | ARG | 109 | −10.550 | 14.537 | 37.515 | 1.00 | 50.91 |
| ATOM | 833 | NH1 | ARG | 109 | −11.810 | 14.363 | 37.886 | 1.00 | 50.39 |
| ATOM | 834 | NH2 | ARG | 109 | −9.640 | 13.643 | 37.868 | 1.00 | 51.02 |
| ATOM | 835 | C | ARG | 109 | −11.497 | 20.076 | 37.508 | 1.00 | 44.07 |
| ATOM | 836 | O | ARG | 109 | −11.536 | 19.484 | 38.582 | 1.00 | 41.18 |
| ATOM | 837 | N | ARG | 110 | −10.836 | 21.214 | 37.357 | 1.00 | 44.70 |
| ATOM | 838 | CA | ARG | 110 | −10.128 | 21.790 | 38.484 | 1.00 | 45.62 |
| ATOM | 839 | CB | ARG | 110 | −9.532 | 23.145 | 38.109 | 1.00 | 49.45 |
| ATOM | 840 | CG | ARG | 110 | −8.769 | 23.823 | 39.239 | 1.00 | 55.43 |
| ATOM | 841 | CD | ARG | 110 | −7.813 | 24.933 | 38.761 | 1.00 | 58.14 |
| ATOM | 842 | NE | ARG | 110 | −8.383 | 25.798 | 37.729 | 1.00 | 59.04 |
| ATOM | 843 | CZ | ARG | 110 | −8.436 | 25.487 | 36.435 | 1.00 | 61.75 |
| ATOM | 844 | NH1 | ARG | 110 | −7.945 | 24.328 | 36.006 | 1.00 | 61.67 |
| ATOM | 845 | NH2 | ARG | 110 | −8.993 | 26.331 | 35.572 | 1.00 | 61.81 |
| ATOM | 846 | C | ARG | 110 | −11.072 | 21.920 | 39.667 | 1.00 | 45.36 |
| ATOM | 847 | O | ARG | 110 | −10.697 | 21.602 | 40.804 | 1.00 | 44.82 |
| ATOM | 848 | N | LYS | 111 | −12.300 | 22.363 | 39.404 | 1.00 | 44.64 |
| ATOM | 849 | CA | LYS | 111 | −13.283 | 22.500 | 40.476 | 1.00 | 46.68 |
| ATOM | 850 | CB | LYS | 111 | −14.549 | 23.225 | 39.997 | 1.00 | 48.26 |
| ATOM | 851 | CG | LYS | 111 | −14.313 | 24.695 | 39.665 | 1.00 | 53.39 |
| ATOM | 852 | CD | LYS | 111 | −15.621 | 25.451 | 39.447 | 1.00 | 56.17 |
| ATOM | 853 | CE | LYS | 111 | −15.382 | 26.917 | 39.046 | 1.00 | 58.69 |
| ATOM | 854 | NZ | LYS | 111 | −14.714 | 27.105 | 37.709 | 1.00 | 56.88 |
| ATOM | 855 | C | LYS | 111 | −13.640 | 21.138 | 41.067 | 1.00 | 44.82 |
| ATOM | 856 | O | LYS | 111 | −13.726 | 21.010 | 42.276 | 1.00 | 44.23 |
| ATOM | 857 | N | ASP | 112 | −13.841 | 20.125 | 40.231 | 1.00 | 45.41 |
| ATOM | 858 | CA | ASP | 112 | −14.141 | 18.793 | 40.762 | 1.00 | 47.37 |
| ATOM | 859 | CB | ASP | 112 | −14.362 | 17.766 | 39.634 | 1.00 | 49.39 |
| ATOM | 860 | CG | ASP | 112 | −15.388 | 18.222 | 38.599 | 1.00 | 56.37 |
| ATOM | 861 | OD1 | ASP | 112 | −16.549 | 18.548 | 38.976 | 1.00 | 58.22 |
| ATOM | 862 | OD2 | ASP | 112 | −15.030 | 18.245 | 37.388 | 1.00 | 58.89 |
| ATOM | 863 | C | ASP | 112 | −12.966 | 18.304 | 41.657 | 1.00 | 46.29 |
| ATOM | 864 | O | ASP | 112 | −13.179 | 17.652 | 42.672 | 1.00 | 47.08 |
| ATOM | 865 | N | LEU | 113 | −11.728 | 18.622 | 41.288 | 1.00 | 42.72 |
| ATOM | 866 | CA | LEU | 113 | −10.582 | 18.181 | 42.084 | 1.00 | 40.11 |
| ATOM | 867 | CB | LEU | 113 | −9.323 | 18.172 | 41.222 | 1.00 | 37.15 |
| ATOM | 868 | CG | LEU | 113 | −9.390 | 17.125 | 40.111 | 1.00 | 36.21 |
| ATOM | 869 | CD1 | LEU | 113 | −8.274 | 17.320 | 39.106 | 1.00 | 36.61 |
| ATOM | 870 | CD2 | LEU | 113 | −9.341 | 15.744 | 40.742 | 1.00 | 37.64 |
| ATOM | 871 | C | LEU | 113 | −10.370 | 19.027 | 43.340 | 1.00 | 39.82 |
| ATOM | 872 | O | LEU | 113 | −9.973 | 18.509 | 44.400 | 1.00 | 37.85 |
| ATOM | 873 | N | THR | 114 | −10.638 | 20.325 | 43.228 | 1.00 | 38.24 |
| ATOM | 874 | CA | THR | 114 | −10.479 | 21.206 | 44.375 | 1.00 | 37.09 |
| ATOM | 875 | CB | THR | 114 | −10.828 | 22.666 | 44.019 | 1.00 | 36.57 |
| ATOM | 876 | OG1 | THR | 114 | −9.924 | 23.153 | 43.017 | 1.00 | 33.29 |
| ATOM | 877 | CG2 | THR | 114 | −10.723 | 23.547 | 45.252 | 1.00 | 37.73 |
| ATOM | 878 | C | THR | 114 | −11.459 | 20.708 | 45.417 | 1.00 | 39.56 |
| ATOM | 879 | O | THR | 114 | −11.163 | 20.652 | 46.611 | 1.00 | 37.29 |
| ATOM | 880 | N | LYS | 115 | −12.638 | 20.319 | 44.946 | 1.00 | 40.54 |
| ATOM | 881 | CA | LYS | 115 | −13.661 | 19.842 | 45.848 | 1.00 | 41.37 |
| ATOM | 882 | CB | LYS | 115 | −14.974 | 19.666 | 45.083 | 1.00 | 45.84 |
| ATOM | 883 | CG | LYS | 115 | −16.101 | 19.059 | 45.880 | 1.00 | 49.09 |
| ATOM | 884 | CD | LYS | 115 | −17.418 | 19.248 | 45.151 | 1.00 | 53.42 |
| ATOM | 885 | CE | LYS | 115 | −18.522 | 18.381 | 45.760 | 1.00 | 56.03 |
| ATOM | 886 | NZ | LYS | 115 | −18.314 | 16.917 | 45.456 | 1.00 | 57.60 |
| ATOM | 887 | C | LYS | 115 | −13.242 | 18.561 | 46.542 | 1.00 | 38.50 |
| ATOM | 888 | O | LYS | 115 | −13.495 | 18.382 | 47.720 | 1.00 | 37.67 |
| ATOM | 889 | N | ILE | 116 | −12.560 | 17.685 | 45.823 | 1.00 | 38.50 |
| ATOM | 890 | CA | ILE | 116 | −12.133 | 16.434 | 46.419 | 1.00 | 37.98 |
| ATOM | 891 | CB | ILE | 116 | −11.617 | 15.452 | 45.374 | 1.00 | 39.26 |
| ATOM | 892 | CG2 | ILE | 116 | −11.367 | 14.104 | 46.027 | 1.00 | 39.71 |
| ATOM | 893 | CG1 | ILE | 116 | −12.640 | 15.297 | 44.257 | 1.00 | 39.44 |
| ATOM | 894 | CD1 | ILE | 116 | −13.980 | 14.876 | 44.742 | 1.00 | 42.71 |
| ATOM | 895 | C | ILE | 116 | −11.050 | 16.602 | 47.474 | 1.00 | 37.87 |
| ATOM | 896 | O | ILE | 116 | −11.127 | 15.979 | 48.537 | 1.00 | 38.93 |
| ATOM | 897 | N | VAL | 117 | −10.041 | 17.425 | 47.209 | 1.00 | 35.17 |
| ATOM | 898 | CA | VAL | 117 | −8.996 | 17.584 | 48.220 | 1.00 | 35.04 |
| ATOM | 899 | CB | VAL | 117 | −7.764 | 18.330 | 47.666 | 1.00 | 32.48 |
| ATOM | 900 | CG1 | VAL | 117 | −7.220 | 17.566 | 46.481 | 1.00 | 33.10 |
| ATOM | 901 | CG2 | VAL | 117 | −8.123 | 19.714 | 47.248 | 1.00 | 32.62 |
| ATOM | 902 | C | VAL | 117 | −9.560 | 18.268 | 49.468 | 1.00 | 33.43 |
| ATOM | 903 | O | VAL | 117 | −9.215 | 17.897 | 50.596 | 1.00 | 33.84 |
| ATOM | 904 | N | ARG | 118 | −10.442 | 19.246 | 49.274 | 1.00 | 33.17 |
| ATOM | 905 | CA | ARG | 118 | −11.075 | 19.918 | 50.419 | 1.00 | 33.39 |
| ATOM | 906 | CB | ARG | 118 | −11.982 | 21.060 | 49.961 | 1.00 | 29.92 |
| ATOM | 907 | CG | ARG | 118 | −11.192 | 22.288 | 49.649 | 1.00 | 32.89 |
| ATOM | 908 | CD | ARG | 118 | −12.029 | 23.334 | 49.036 | 1.00 | 37.88 |
| ATOM | 909 | NE | ARG | 118 | −11.219 | 24.462 | 48.622 | 1.00 | 41.77 |
| ATOM | 910 | CZ | ARG | 118 | −11.695 | 25.532 | 48.003 | 1.00 | 46.19 |
| ATOM | 911 | NH1 | ARG | 118 | −12.987 | 25.626 | 47.718 | 1.00 | 47.00 |
| ATOM | 912 | NH2 | ARG | 118 | −10.871 | 26.517 | 47.679 | 1.00 | 49.71 |
| ATOM | 913 | C | ARG | 118 | −11.859 | 18.945 | 51.297 | 1.00 | 32.57 |
| ATOM | 914 | O | ARG | 118 | −11.806 | 19.046 | 52.515 | 1.00 | 36.89 |
| ATOM | 915 | N | GLY | 119 | −12.570 | 18.008 | 50.677 | 1.00 | 30.46 |
| ATOM | 916 | CA | GLY | 119 | −13.322 | 17.018 | 51.423 | 1.00 | 29.23 |
| ATOM | 917 | C | GLY | 119 | −12.407 | 16.082 | 52.202 | 1.00 | 29.62 |
| ATOM | 918 | O | GLY | 119 | −12.702 | 15.722 | 53.335 | 1.00 | 30.06 |
| ATOM | 919 | N | GLU | 120 | −11.294 | 15.692 | 51.593 | 1.00 | 30.52 |
| ATOM | 920 | CA | GLU | 120 | −10.321 | 14.827 | 52.241 | 1.00 | 31.96 |
| ATOM | 921 | CB | GLU | 120 | −9.281 | 14.349 | 51.222 | 1.00 | 34.72 |
| ATOM | 922 | CG | GLU | 120 | −9.912 | 13.483 | 50.152 | 1.00 | 39.64 |
| ATOM | 923 | CD | GLU | 120 | −8.962 | 13.047 | 49.049 | 1.00 | 42.12 |
| ATOM | 924 | OE1 | GLU | 120 | −9.414 | 12.270 | 48.184 | 1.00 | 44.99 |
| ATOM | 925 | OE2 | GLU | 120 | −7.780 | 13.461 | 49.030 | 1.00 | 43.53 |
| ATOM | 926 | C | GLU | 120 | −9.649 | 15.592 | 53.368 | 1.00 | 32.43 |
| ATOM | 927 | O | GLU | 120 | −9.499 | 15.063 | 54.462 | 1.00 | 35.07 |
| ATOM | 928 | N | ALA | 121 | −9.260 | 16.845 | 53.124 | 1.00 | 32.32 |
| ATOM | 929 | CA | ALA | 121 | −8.631 | 17.616 | 54.186 | 1.00 | 31.62 |
| ATOM | 930 | CB | ALA | 121 | −8.336 | 19.035 | 53.720 | 1.00 | 28.12 |
| ATOM | 931 | C | ALA | 121 | −9.624 | 17.634 | 55.344 | 1.00 | 34.83 |
| ATOM | 932 | O | ALA | 121 | −9.293 | 17.273 | 56.475 | 1.00 | 36.01 |
| ATOM | 933 | N | GLU | 122 | −10.858 | 18.018 | 55.026 | 1.00 | 35.31 |
| ATOM | 934 | CA | GLU | 122 | −11.944 | 18.115 | 55.992 | 1.00 | 36.50 |
| ATOM | 935 | CB | GLU | 122 | −13.259 | 18.348 | 55.242 | 1.00 | 39.77 |
| ATOM | 936 | CG | GLU | 122 | −14.201 | 19.288 | 55.924 | 1.00 | 39.83 |
| ATOM | 937 | CD | GLU | 122 | −14.446 | 18.903 | 57.343 | 1.00 | 40.58 |
| ATOM | 938 | OE1 | GLU | 122 | −14.903 | 17.768 | 57.553 | 1.00 | 44.91 |
| ATOM | 939 | OE2 | GLU | 122 | −14.184 | 19.728 | 58.250 | 1.00 | 41.71 |
| ATOM | 940 | C | GLU | 122 | −12.028 | 16.836 | 56.808 | 1.00 | 36.15 |
| ATOM | 941 | O | GLU | 122 | −12.081 | 16.875 | 58.037 | 1.00 | 34.04 |
| ATOM | 942 | N | GLN | 123 | −12.053 | 15.698 | 56.115 | 1.00 | 36.64 |
| ATOM | 943 | CA | GLN | 123 | −12.099 | 14.413 | 56.791 | 1.00 | 38.70 |
| ATOM | 944 | CB | GLN | 123 | −12.070 | 13.295 | 55.751 | 1.00 | 40.64 |
| ATOM | 945 | CG | GLN | 123 | −11.999 | 11.888 | 56.331 | 1.00 | 45.87 |
| ATOM | 946 | CD | GLN | 123 | −10.575 | 11.398 | 56.525 | 1.00 | 50.88 |
| ATOM | 947 | OE1 | GLN | 123 | −9.774 | 11.378 | 55.579 | 1.00 | 54.39 |
| ATOM | 948 | NE2 | GLN | 123 | −10.254 | 10.983 | 57.739 | 1.00 | 50.01 |
| ATOM | 949 | C | GLN | 123 | −10.910 | 14.277 | 57.760 | 1.00 | 37.35 |
| ATOM | 950 | O | GLN | 123 | −11.070 | 13.855 | 58.900 | 1.00 | 37.65 |
| ATOM | 951 | N | ALA | 124 | −9.721 | 14.652 | 57.296 | 1.00 | 35.43 |
| ATOM | 952 | CA | ALA | 124 | −8.522 | 14.576 | 58.123 | 1.00 | 34.84 |
| ATOM | 953 | CB | ALA | 124 | −7.252 | 15.024 | 57.307 | 1.00 | 33.06 |
| ATOM | 954 | C | ALA | 124 | −8.670 | 15.431 | 59.384 | 1.00 | 32.69 |
| ATOM | 955 | O | ALA | 124 | −8.236 | 15.024 | 60.470 | 1.00 | 31.59 |
| ATOM | 956 | N | ARG | 125 | −9.266 | 16.612 | 59.237 | 1.00 | 29.13 |
| ATOM | 957 | CA | ARG | 125 | −9.464 | 17.488 | 60.377 | 1.00 | 30.40 |
| ATOM | 958 | CB | ARG | 125 | −9.883 | 18.901 | 59.943 | 1.00 | 33.05 |
| ATOM | 959 | CG | ARG | 125 | −8.734 | 19.821 | 59.545 | 1.00 | 36.01 |
| ATOM | 960 | CD | ARG | 125 | −9.244 | 21.249 | 59.398 | 1.00 | 37.50 |
| ATOM | 961 | NE | ARG | 125 | −10.102 | 21.309 | 58.240 | 1.00 | 41.70 |
| ATOM | 962 | CZ | ARG | 125 | −9.683 | 21.653 | 57.037 | 1.00 | 39.98 |
| ATOM | 963 | NH1 | ARG | 125 | −8.415 | 21.979 | 56.856 | 1.00 | 42.73 |
| ATOM | 964 | NH2 | ARG | 125 | −10.502 | 21.568 | 56.007 | 1.00 | 37.33 |
| ATOM | 965 | C | ARG | 125 | −10.468 | 16.943 | 61.386 | 1.00 | 30.54 |
| ATOM | 966 | O | ARG | 125 | −10.296 | 17.144 | 62.580 | 1.00 | 31.78 |
| ATOM | 967 | N | VAL | 126 | −11.511 | 16.262 | 60.923 | 1.00 | 30.13 |
| ATOM | 968 | CA | VAL | 126 | −12.474 | 15.683 | 61.847 | 1.00 | 29.18 |
| ATOM | 969 | CB | VAL | 126 | −13.762 | 15.137 | 61.107 | 1.00 | 30.93 |
| ATOM | 970 | CG1 | VAL | 126 | −14.721 | 14.517 | 62.104 | 1.00 | 26.39 |
| ATOM | 971 | CG2 | VAL | 126 | −14.509 | 16.292 | 60.354 | 1.00 | 28.70 |
| ATOM | 972 | C | VAL | 126 | −11.754 | 14.531 | 62.583 | 1.00 | 30.04 |
| ATOM | 973 | O | VAL | 126 | −11.820 | 14.422 | 63.816 | 1.00 | 31.66 |
| ATOM | 974 | N | ALA | 127 | −11.031 | 13.698 | 61.849 | 1.00 | 28.96 |
| ATOM | 975 | CA | ALA | 127 | −10.337 | 12.583 | 62.492 | 1.00 | 30.05 |
| ATOM | 976 | CB | ALA | 127 | −9.574 | 11.753 | 61.434 | 1.00 | 29.30 |
| ATOM | 977 | C | ALA | 127 | −9.374 | 13.067 | 63.589 | 1.00 | 29.90 |
| ATOM | 978 | O | ALA | 127 | −9.308 | 12.493 | 64.673 | 1.00 | 31.73 |
| ATOM | 979 | N | VAL | 128 | −8.643 | 14.137 | 63.312 | 1.00 | 31.82 |
| ATOM | 980 | CA | VAL | 128 | −7.686 | 14.694 | 64.264 | 1.00 | 31.72 |
| ATOM | 981 | CB | VAL | 128 | −6.844 | 15.819 | 63.581 | 1.00 | 33.25 |
| ATOM | 982 | CG1 | VAL | 128 | −5.997 | 16.559 | 64.626 | 1.00 | 35.15 |
| ATOM | 983 | CG2 | VAL | 128 | −5.936 | 15.195 | 62.504 | 1.00 | 28.98 |
| ATOM | 984 | C | VAL | 128 | −8.346 | 15.229 | 65.546 | 1.00 | 34.61 |
| ATOM | 985 | O | VAL | 128 | −7.795 | 15.063 | 66.644 | 1.00 | 34.11 |
| ATOM | 986 | N | ARG | 129 | −9.514 | 15.863 | 65.396 | 1.00 | 34.97 |
| ATOM | 987 | CA | ARG | 129 | −10.285 | 16.415 | 66.518 | 1.00 | 36.61 |
| ATOM | 988 | CB | ARG | 129 | −11.416 | 17.335 | 66.048 | 1.00 | 39.01 |
| ATOM | 989 | CG | ARG | 129 | −11.084 | 18.404 | 65.030 | 1.00 | 42.15 |
| ATOM | 990 | CD | ARG | 129 | −12.401 | 18.970 | 64.452 | 1.00 | 43.95 |
| ATOM | 991 | NE | ARG | 129 | −12.193 | 19.869 | 63.321 | 1.00 | 46.07 |
| ATOM | 992 | CZ | ARG | 129 | −11.349 | 20.900 | 63.338 | 1.00 | 47.32 |
| ATOM | 993 | NH1 | ARG | 129 | −10.623 | 21.162 | 64.426 | 1.00 | 47.90 |
| ATOM | 994 | NH2 | ARG | 129 | −11.245 | 21.682 | 62.284 | 1.00 | 44.26 |
| ATOM | 995 | C | ARG | 129 | −10.964 | 15.279 | 67.291 | 1.00 | 36.52 |
| ATOM | 996 | O | ARG | 129 | −11.378 | 15.471 | 68.421 | 1.00 | 37.01 |
| ATOM | 997 | N | ASN | 130 | −11.146 | 14.122 | 66.664 | 1.00 | 35.22 |
| ATOM | 998 | CA | ASN | 130 | −11.762 | 13.006 | 67.376 | 1.00 | 34.87 |
| ATOM | 999 | CB | ASN | 130 | −12.340 | 11.970 | 66.367 | 1.00 | 33.94 |
| ATOM | 1000 | CG | ASN | 130 | −13.708 | 12.406 | 65.788 | 1.00 | 34.78 |
| ATOM | 1001 | OD1 | ASN | 130 | −14.138 | 11.953 | 64.723 | 1.00 | 32.62 |
| ATOM | 1002 | ND2 | ASN | 130 | −14.386 | 13.282 | 66.507 | 1.00 | 29.90 |
| ATOM | 1003 | C | ASN | 130 | −10.679 | 12.394 | 68.285 | 1.00 | 36.22 |
| ATOM | 1004 | O | ASN | 130 | −10.954 | 11.958 | 69.403 | 1.00 | 35.32 |
| ATOM | 1005 | N | VAL | 131 | −9.439 | 12.378 | 67.803 | 1.00 | 37.20 |
| ATOM | 1006 | CA | VAL | 131 | −8.359 | 11.849 | 68.607 | 1.00 | 38.51 |
| ATOM | 1007 | CB | VAL | 131 | −7.044 | 11.745 | 67.811 | 1.00 | 38.07 |
| ATOM | 1008 | CG1 | VAL | 131 | −5.916 | 11.279 | 68.733 | 1.00 | 38.68 |
| ATOM | 1009 | CG2 | VAL | 131 | −7.215 | 10.797 | 66.651 | 1.00 | 37.75 |
| ATOM | 1010 | C | VAL | 131 | −8.148 | 12.816 | 69.782 | 1.00 | 40.98 |
| ATOM | 1011 | O | VAL | 131 | −8.009 | 12.364 | 70.930 | 1.00 | 40.00 |
| ATOM | 1012 | N | ARG | 132 | −8.141 | 14.130 | 69.499 | 1.00 | 40.45 |
| ATOM | 1013 | CA | ARG | 132 | −7.943 | 15.133 | 70.547 | 1.00 | 42.82 |
| ATOM | 1014 | CB | ARG | 132 | −8.178 | 16.559 | 70.046 | 1.00 | 42.45 |
| ATOM | 1015 | CG | ARG | 132 | −8.001 | 17.603 | 71.165 | 1.00 | 48.52 |
| ATOM | 1016 | CD | ARG | 132 | −8.630 | 19.001 | 70.918 | 1.00 | 50.87 |
| ATOM | 1017 | NE | ARG | 132 | −10.080 | 18.961 | 71.107 | 1.00 | 57.79 |
| ATOM | 1018 | CZ | ARG | 132 | −10.961 | 18.537 | 70.198 | 1.00 | 59.32 |
| ATOM | 1019 | NH1 | ARG | 132 | −10.562 | 18.114 | 69.011 | 1.00 | 62.98 |
| ATOM | 1020 | NH2 | ARG | 132 | −12.246 | 18.467 | 70.501 | 1.00 | 59.20 |
| ATOM | 1021 | C | ARG | 132 | −8.935 | 14.855 | 71.658 | 1.00 | 44.60 |
| ATOM | 1022 | O | ARG | 132 | −8.572 | 14.723 | 72.828 | 1.00 | 42.61 |
| ATOM | 1023 | N | ARG | 133 | −10.196 | 14.767 | 71.258 | 1.00 | 46.77 |
| ATOM | 1024 | CA | ARG | 133 | −11.300 | 14.497 | 72.162 | 1.00 | 50.55 |
| ATOM | 1025 | CB | ARG | 133 | −12.582 | 14.317 | 71.351 | 1.00 | 52.21 |
| ATOM | 1026 | CG | ARG | 133 | −13.848 | 14.227 | 72.170 | 1.00 | 57.48 |
| ATOM | 1027 | CD | ARG | 133 | −15.000 | 13.921 | 71.244 | 1.00 | 63.69 |
| ATOM | 1028 | NE | ARG | 133 | −14.973 | 14.767 | 70.047 | 1.00 | 68.51 |
| ATOM | 1029 | CZ | ARG | 133 | −15.071 | 16.097 | 70.051 | 1.00 | 70.24 |
| ATOM | 1030 | NH1 | ARG | 133 | −15.206 | 16.754 | 71.194 | 1.00 | 72.00 |
| ATOM | 1031 | NH2 | ARG | 133 | −15.030 | 16.772 | 68.908 | 1.00 | 70.13 |
| ATOM | 1032 | C | ARG | 133 | −11.035 | 13.233 | 72.985 | 1.00 | 52.21 |
| ATOM | 1033 | O | ARG | 133 | −11.082 | 13.265 | 74.218 | 1.00 | 51.81 |
| ATOM | 1034 | N | ASP | 134 | −10.763 | 12.123 | 72.302 | 1.00 | 52.59 |
| ATOM | 1035 | CA | ASP | 134 | −10.505 | 10.868 | 72.989 | 1.00 | 54.66 |
| ATOM | 1036 | CB | ASP | 134 | −10.116 | 9.772 | 72.003 | 1.00 | 56.25 |
| ATOM | 1037 | CG | ASP | 134 | −9.959 | 8.427 | 72.682 | 1.00 | 59.16 |
| ATOM | 1038 | OD1 | ASP | 134 | −10.953 | 7.972 | 73.290 | 1.00 | 60.25 |
| ATOM | 1039 | OD2 | ASP | 134 | −8.860 | 7.830 | 72.622 | 1.00 | 58.00 |
| ATOM | 1040 | C | ASP | 134 | −9.404 | 11.007 | 74.028 | 1.00 | 55.18 |
| ATOM | 1041 | O | ASP | 134 | −9.400 | 10.306 | 75.036 | 1.00 | 54.08 |
| ATOM | 1042 | N | ALA | 135 | −8.454 | 11.895 | 73.768 | 1.00 | 56.31 |
| ATOM | 1043 | CA | ALA | 135 | −7.369 | 12.133 | 74.714 | 1.00 | 57.14 |
| ATOM | 1044 | CB | ALA | 135 | −6.267 | 12.944 | 74.057 | 1.00 | 55.72 |
| ATOM | 1045 | C | ALA | 135 | −7.954 | 12.902 | 75.896 | 1.00 | 57.58 |
| ATOM | 1046 | O | ALA | 135 | −7.872 | 12.462 | 77.040 | 1.00 | 57.83 |
| ATOM | 1047 | N | ASN | 136 | −8.549 | 14.053 | 75.599 | 1.00 | 59.12 |
| ATOM | 1048 | CA | ASN | 136 | −9.176 | 14.900 | 76.610 | 1.00 | 61.05 |
| ATOM | 1049 | CB | ASN | 136 | −10.100 | 15.940 | 75.955 | 1.00 | 60.54 |
| ATOM | 1050 | CG | ASN | 136 | −9.392 | 16.808 | 74.912 | 1.00 | 62.07 |
| ATOM | 1051 | OD1 | ASN | 136 | −8.439 | 17.531 | 75.220 | 1.00 | 62.74 |
| ATOM | 1052 | ND2 | ASN | 136 | −9.870 | 16.748 | 73.673 | 1.00 | 61.09 |
| ATOM | 1053 | C | ASN | 136 | −10.007 | 14.036 | 77.565 | 1.00 | 62.37 |
| ATOM | 1054 | O | ASN | 136 | −9.965 | 14.233 | 78.773 | 1.00 | 63.28 |
| ATOM | 1055 | N | ASP | 137 | −10.758 | 13.084 | 77.013 | 1.00 | 64.25 |
| ATOM | 1056 | CA | ASP | 137 | −11.601 | 12.191 | 77.812 | 1.00 | 66.76 |
| ATOM | 1057 | CB | ASP | 137 | −12.272 | 11.131 | 76.940 | 1.00 | 67.37 |
| ATOM | 1058 | CG | ASP | 137 | −13.250 | 11.714 | 75.967 | 1.00 | 68.71 |
| ATOM | 1059 | OD1 | ASP | 137 | −14.119 | 12.507 | 76.398 | 1.00 | 68.32 |
| ATOM | 1060 | OD2 | ASP | 137 | −13.156 | 11.359 | 74.772 | 1.00 | 70.08 |
| ATOM | 1061 | C | ASP | 137 | −10.821 | 11.462 | 78.889 | 1.00 | 67.84 |
| ATOM | 1062 | O | ASP | 137 | −11.079 | 11.616 | 80.081 | 1.00 | 68.36 |
| ATOM | 1063 | N | LYS | 138 | −9.887 | 10.634 | 78.449 | 1.00 | 68.15 |
| ATOM | 1064 | CA | LYS | 138 | −9.060 | 9.875 | 79.362 | 1.00 | 70.26 |
| ATOM | 1065 | CB | LYS | 138 | −7.922 | 9.208 | 78.583 | 1.00 | 68.44 |
| ATOM | 1066 | CG | LYS | 138 | −8.419 | 8.213 | 77.552 | 1.00 | 67.37 |
| ATOM | 1067 | CD | LYS | 138 | −7.279 | 7.532 | 76.846 | 1.00 | 66.77 |
| ATOM | 1068 | CE | LYS | 138 | −7.794 | 6.509 | 75.855 | 1.00 | 65.32 |
| ATOM | 1069 | NZ | LYS | 138 | −6.661 | 5.798 | 75.206 | 1.00 | 65.96 |
| ATOM | 1070 | C | LYS | 138 | −8.509 | 10.791 | 80.454 | 1.00 | 72.20 |
| ATOM | 1071 | O | LYS | 138 | −8.637 | 10.492 | 81.642 | 1.00 | 71.61 |
| ATOM | 1072 | N | VAL | 139 | −7.919 | 11.910 | 80.037 | 1.00 | 73.71 |
| ATOM | 1073 | CA | VAL | 139 | −7.341 | 12.885 | 80.955 | 1.00 | 75.93 |
| ATOM | 1074 | CB | VAL | 139 | −6.746 | 14.094 | 80.181 | 1.00 | 75.79 |
| ATOM | 1075 | CG1 | VAL | 139 | −6.124 | 15.085 | 81.160 | 1.00 | 75.22 |
| ATOM | 1076 | CG2 | VAL | 139 | −5.708 | 13.615 | 79.168 | 1.00 | 74.05 |
| ATOM | 1077 | C | VAL | 139 | −8.357 | 13.416 | 81.974 | 1.00 | 77.78 |
| ATOM | 1078 | O | VAL | 139 | −8.168 | 13.284 | 83.184 | 1.00 | 77.15 |
| ATOM | 1079 | N | LYS | 140 | −9.432 | 14.017 | 81.471 | 1.00 | 80.32 |
| ATOM | 1080 | CA | LYS | 140 | −10.486 | 14.591 | 82.309 | 1.00 | 82.58 |
| ATOM | 1081 | CB | LYS | 140 | −11.531 | 15.266 | 81.413 | 1.00 | 83.61 |
| ATOM | 1082 | CG | LYS | 140 | −12.436 | 16.253 | 82.117 | 1.00 | 84.66 |
| ATOM | 1083 | CD | LYS | 140 | −11.616 | 17.410 | 82.677 | 1.00 | 86.11 |
| ATOM | 1084 | CE | LYS | 140 | −12.504 | 18.499 | 83.265 | 1.00 | 87.30 |
| ATOM | 1085 | NZ | LYS | 140 | −13.401 | 18.007 | 84.343 | 1.00 | 87.20 |
| ATOM | 1086 | C | LYS | 140 | −11.161 | 13.524 | 83.185 | 1.00 | 83.58 |
| ATOM | 1087 | O | LYS | 140 | −11.543 | 13.788 | 84.329 | 1.00 | 82.45 |
| ATOM | 1088 | N | ALA | 141 | −11.297 | 12.321 | 82.633 | 1.00 | 85.31 |
| ATOM | 1089 | CA | ALA | 141 | −11.917 | 11.201 | 83.331 | 1.00 | 87.36 |
| ATOM | 1090 | CB | ALA | 141 | −12.222 | 10.079 | 82.349 | 1.00 | 87.57 |
| ATOM | 1091 | C | ALA | 141 | −11.022 | 10.676 | 84.442 | 1.00 | 88.79 |
| ATOM | 1092 | O | ALA | 141 | −11.479 | 10.439 | 85.555 | 1.00 | 89.29 |
| ATOM | 1093 | N | LEU | 142 | −9.745 | 10.492 | 84.130 | 1.00 | 90.16 |
| ATOM | 1094 | CA | LEU | 142 | −8.781 | 9.982 | 85.098 | 1.00 | 91.17 |
| ATOM | 1095 | CB | LEU | 142 | −7.390 | 9.942 | 84.467 | 1.00 | 90.19 |
| ATOM | 1096 | CG | LEU | 142 | −6.450 | 8.865 | 84.999 | 1.00 | 90.65 |
| ATOM | 1097 | CD1 | LEU | 142 | −7.081 | 7.503 | 84.763 | 1.00 | 90.25 |
| ATOM | 1098 | CD2 | LEU | 142 | −5.108 | 8.947 | 84.292 | 1.00 | 90.32 |
| ATOM | 1099 | C | LEU | 142 | −8.782 | 10.889 | 86.330 | 1.00 | 92.69 |
| ATOM | 1100 | O | LEU | 142 | −8.453 | 10.463 | 87.439 | 1.00 | 92.75 |
| ATOM | 1101 | N | LEU | 143 | −9.175 | 12.141 | 86.120 | 1.00 | 94.14 |
| ATOM | 1102 | CA | LEU | 143 | −9.243 | 13.134 | 87.188 | 1.00 | 95.26 |
| ATOM | 1103 | CB | LEU | 143 | −9.537 | 14.513 | 86.585 | 1.00 | 94.57 |
| ATOM | 1104 | CG | LEU | 143 | −9.684 | 15.706 | 87.531 | 1.00 | 93.49 |
| ATOM | 1105 | CD1 | LEU | 143 | −8.373 | 15.956 | 88.248 | 1.00 | 93.46 |
| ATOM | 1106 | CD2 | LEU | 143 | −10.091 | 16.933 | 86.735 | 1.00 | 92.61 |
| ATOM | 1107 | C | LEU | 143 | −10.324 | 12.789 | 88.212 | 1.00 | 96.21 |
| ATOM | 1108 | O | LEU | 143 | −10.246 | 13.195 | 89.370 | 1.00 | 95.47 |
| ATOM | 1109 | N | LYS | 144 | −11.331 | 12.036 | 87.778 | 1.00 | 98.51 |
| ATOM | 1110 | CA | LYS | 144 | −12.441 | 11.662 | 88.649 | 1.00 | 100.82 |
| ATOM | 1111 | CB | LYS | 144 | −13.506 | 10.862 | 87.880 | 1.00 | 100.58 |
| ATOM | 1112 | CG | LYS | 144 | −13.063 | 9.460 | 87.477 | 1.00 | 101.19 |
| ATOM | 1113 | CD | LYS | 144 | −14.161 | 8.663 | 86.800 | 1.00 | 101.64 |
| ATOM | 1114 | CE | LYS | 144 | −13.669 | 7.259 | 86.448 | 1.00 | 102.30 |
| ATOM | 1115 | NZ | LYS | 144 | −14.711 | 6.420 | 85.783 | 1.00 | 102.31 |
| ATOM | 1116 | C | LYS | 144 | −12.002 | 10.847 | 89.854 | 1.00 | 102.32 |
| ATOM | 1117 | O | LYS | 144 | −12.148 | 11.296 | 90.990 | 1.00 | 102.94 |
| ATOM | 1118 | N | ASP | 145 | −11.459 | 9.657 | 89.601 | 1.00 | 103.95 |
| ATOM | 1119 | CA | ASP | 145 | −11.042 | 8.758 | 90.672 | 1.00 | 105.61 |
| ATOM | 1120 | CB | ASP | 145 | −11.670 | 7.364 | 90.449 | 1.00 | 105.33 |
| ATOM | 1121 | CG | ASP | 145 | −13.200 | 7.379 | 90.493 | 1.00 | 105.16 |
| ATOM | 1122 | OD1 | ASP | 145 | −13.778 | 7.740 | 91.545 | 1.00 | 104.28 |
| ATOM | 1123 | OD2 | ASP | 145 | −13.827 | 7.028 | 89.472 | 1.00 | 104.93 |
| ATOM | 1124 | C | ASP | 145 | −9.533 | 8.600 | 90.897 | 1.00 | 106.36 |
| ATOM | 1125 | O | ASP | 145 | −9.071 | 8.609 | 92.040 | 1.00 | 106.84 |
| ATOM | 1126 | N | LYS | 146 | −8.764 | 8.475 | 89.817 | 1.00 | 107.05 |
| ATOM | 1127 | CA | LYS | 146 | −7.323 | 8.273 | 89.963 | 1.00 | 107.59 |
| ATOM | 1128 | CB | LYS | 146 | −6.829 | 7.347 | 88.840 | 1.00 | 107.69 |
| ATOM | 1129 | CG | LYS | 146 | −7.534 | 5.979 | 88.885 | 1.00 | 108.01 |
| ATOM | 1130 | CD | LYS | 146 | −6.967 | 4.952 | 87.912 | 1.00 | 107.88 |
| ATOM | 1131 | CE | LYS | 146 | −7.653 | 3.595 | 88.104 | 1.00 | 107.75 |
| ATOM | 1132 | NZ | LYS | 146 | −7.039 | 2.522 | 87.273 | 1.00 | 107.65 |
| ATOM | 1133 | C | LYS | 146 | −6.449 | 9.527 | 90.091 | 1.00 | 108.00 |
| ATOM | 1134 | O | LYS | 146 | −6.909 | 10.654 | 89.905 | 1.00 | 108.57 |
| ATOM | 1135 | N | GLU | 147 | −5.183 | 9.286 | 90.415 | 1.00 | 107.89 |
| ATOM | 1136 | CA | GLU | 147 | −4.118 | 10.269 | 90.678 | 1.00 | 107.41 |
| ATOM | 1137 | CB | GLU | 147 | −2.780 | 9.531 | 90.640 | 1.00 | 108.68 |
| ATOM | 1138 | CG | GLU | 147 | −2.465 | 8.846 | 89.314 | 1.00 | 109.90 |
| ATOM | 1139 | CD | GLU | 147 | −1.146 | 8.093 | 89.368 | 1.00 | 111.13 |
| ATOM | 1140 | OE1 | GLU | 147 | −1.020 | 7.174 | 90.206 | 1.00 | 111.54 |
| ATOM | 1141 | OE2 | GLU | 147 | −0.235 | 8.421 | 88.579 | 1.00 | 111.87 |
| ATOM | 1142 | C | GLU | 147 | −3.868 | 11.665 | 90.067 | 1.00 | 106.09 |
| ATOM | 1143 | O | GLU | 147 | −3.101 | 12.420 | 90.647 | 1.00 | 107.03 |
| ATOM | 1144 | N | ILE | 148 | −4.448 | 12.040 | 88.936 | 1.00 | 103.57 |
| ATOM | 1145 | CA | ILE | 148 | −4.121 | 13.373 | 88.414 | 1.00 | 101.47 |
| ATOM | 1146 | CB | ILE | 148 | −4.278 | 13.430 | 86.892 | 1.00 | 101.45 |
| ATOM | 1147 | CG2 | ILE | 148 | −3.702 | 14.724 | 86.364 | 1.00 | 101.52 |
| ATOM | 1148 | CG1 | ILE | 148 | −3.553 | 12.249 | 86.245 | 1.00 | 101.43 |
| ATOM | 1149 | CD1 | ILE | 148 | −4.200 | 10.917 | 86.516 | 1.00 | 101.07 |
| ATOM | 1150 | C | ILE | 148 | −4.974 | 14.484 | 89.025 | 1.00 | 100.34 |
| ATOM | 1151 | O | ILE | 148 | −6.083 | 14.222 | 89.495 | 1.00 | 100.42 |
| ATOM | 1152 | N | SER | 149 | −4.463 | 15.717 | 89.021 | 1.00 | 98.53 |
| ATOM | 1153 | CA | SER | 149 | −5.202 | 16.873 | 89.556 | 1.00 | 96.84 |
| ATOM | 1154 | CB | SER | 149 | −4.599 | 17.320 | 90.882 | 1.00 | 97.16 |
| ATOM | 1155 | OG | SER | 149 | −3.248 | 17.689 | 90.706 | 1.00 | 97.28 |
| ATOM | 1156 | C | SER | 149 | −5.203 | 18.049 | 88.579 | 1.00 | 95.62 |
| ATOM | 1157 | O | SER | 149 | −4.454 | 18.056 | 87.608 | 1.00 | 95.10 |
| ATOM | 1158 | N | GLU | 150 | −6.040 | 19.048 | 88.860 | 1.00 | 94.27 |
| ATOM | 1159 | CA | GLU | 150 | −6.215 | 20.244 | 88.028 | 1.00 | 92.89 |
| ATOM | 1160 | CB | GLU | 150 | −6.809 | 21.357 | 88.891 | 1.00 | 92.94 |
| ATOM | 1161 | CG | GLU | 150 | −8.218 | 21.047 | 89.354 | 1.00 | 93.02 |
| ATOM | 1162 | CD | GLU | 150 | −8.668 | 21.948 | 90.477 | 1.00 | 93.68 |
| ATOM | 1163 | OE1 | GLU | 150 | −8.638 | 23.186 | 90.303 | 1.00 | 94.33 |
| ATOM | 1164 | OE2 | GLU | 150 | −9.053 | 21.411 | 91.537 | 1.00 | 93.85 |
| ATOM | 1165 | C | GLU | 150 | −5.050 | 20.810 | 87.203 | 1.00 | 91.59 |
| ATOM | 1166 | O | GLU | 150 | −5.191 | 20.999 | 85.998 | 1.00 | 91.47 |
| ATOM | 1167 | N | ASP | 151 | −3.919 | 21.092 | 87.839 | 1.00 | 90.68 |
| ATOM | 1168 | CA | ASP | 151 | −2.770 | 21.644 | 87.116 | 1.00 | 89.65 |
| ATOM | 1169 | CB | ASP | 151 | −1.743 | 22.205 | 88.091 | 1.00 | 89.29 |
| ATOM | 1170 | CG | ASP | 151 | −2.240 | 23.431 | 88.812 | 1.00 | 89.52 |
| ATOM | 1171 | OD1 | ASP | 151 | −2.566 | 24.421 | 88.129 | 1.00 | 88.83 |
| ATOM | 1172 | OD2 | ASP | 151 | −2.304 | 23.408 | 90.059 | 1.00 | 90.30 |
| ATOM | 1173 | C | ASP | 151 | −2.080 | 20.661 | 86.178 | 1.00 | 88.62 |
| ATOM | 1174 | O | ASP | 151 | −1.342 | 21.065 | 85.284 | 1.00 | 89.06 |
| ATOM | 1175 | N | ASP | 152 | −2.304 | 19.373 | 86.394 | 1.00 | 87.65 |
| ATOM | 1176 | CA | ASP | 152 | −1.707 | 18.363 | 85.535 | 1.00 | 87.45 |
| ATOM | 1177 | CB | ASP | 152 | −1.480 | 17.072 | 86.324 | 1.00 | 89.17 |
| ATOM | 1178 | CG | ASP | 152 | −0.664 | 16.052 | 85.552 | 1.00 | 91.93 |
| ATOM | 1179 | OD1 | ASP | 152 | −1.036 | 15.721 | 84.403 | 1.00 | 93.10 |
| ATOM | 1180 | OD2 | ASP | 152 | 0.351 | 15.577 | 86.102 | 1.00 | 93.68 |
| ATOM | 1181 | C | ASP | 152 | −2.700 | 18.127 | 84.388 | 1.00 | 85.95 |
| ATOM | 1182 | O | ASP | 152 | −2.324 | 17.698 | 83.295 | 1.00 | 85.41 |
| ATOM | 1183 | N | ASP | 153 | −3.969 | 18.422 | 84.670 | 1.00 | 83.73 |
| ATOM | 1184 | CA | ASP | 153 | −5.066 | 18.284 | 83.720 | 1.00 | 80.91 |
| ATOM | 1185 | CB | ASP | 153 | −6.409 | 18.406 | 84.458 | 1.00 | 80.84 |
| ATOM | 1186 | CG | ASP | 153 | −7.619 | 18.190 | 83.552 | 1.00 | 79.88 |
| ATOM | 1187 | OD1 | ASP | 153 | −8.752 | 18.336 | 84.056 | 1.00 | 79.70 |
| ATOM | 1188 | OD2 | ASP | 153 | −7.452 | 17.873 | 82.355 | 1.00 | 79.02 |
| ATOM | 1189 | C | ASP | 153 | −4.907 | 19.431 | 82.740 | 1.00 | 80.07 |
| ATOM | 1190 | O | ASP | 153 | −4.879 | 19.227 | 81.526 | 1.00 | 79.99 |
| ATOM | 1191 | N | ARG | 154 | −4.790 | 20.639 | 83.284 | 1.00 | 77.88 |
| ATOM | 1192 | CA | ARG | 154 | −4.618 | 21.833 | 82.471 | 1.00 | 76.70 |
| ATOM | 1193 | CB | ARG | 154 | −4.318 | 23.040 | 83.370 | 1.00 | 78.40 |
| ATOM | 1194 | CG | ARG | 154 | −5.457 | 23.397 | 84.310 | 1.00 | 81.12 |
| ATOM | 1195 | CD | ARG | 154 | −5.182 | 24.639 | 85.155 | 1.00 | 82.69 |
| ATOM | 1196 | NE | ARG | 154 | −6.336 | 24.979 | 85.989 | 1.00 | 84.80 |
| ATOM | 1197 | CZ | ARG | 154 | −6.376 | 25.999 | 86.843 | 1.00 | 86.51 |
| ATOM | 1198 | NH1 | ARC | 154 | −5.320 | 26.794 | 86.983 | 1.00 | 87.33 |
| ATOM | 1199 | NH2 | ARG | 154 | −7.470 | 26.225 | 87.561 | 1.00 | 86.40 |
| ATOM | 1200 | C | ARG | 154 | −3.498 | 21.658 | 81.442 | 1.00 | 74.53 |
| ATOM | 1201 | O | ARG | 154 | −3.743 | 21.703 | 80.236 | 1.00 | 73.89 |
| ATOM | 1202 | N | ARG | 155 | −2.274 | 21.463 | 81.935 | 1.00 | 72.04 |
| ATOM | 1203 | CA | ARG | 155 | −1.091 | 21.279 | 81.096 | 1.00 | 68.48 |
| ATOM | 1204 | CB | ARG | 155 | 0.122 | 20.941 | 81.964 | 1.00 | 69.87 |
| ATOM | 1205 | CG | ARG | 155 | 0.724 | 22.109 | 82.723 | 1.00 | 71.53 |
| ATOM | 1206 | CD | ARG | 155 | 1.847 | 21.617 | 83.630 | 1.00 | 73.51 |
| ATOM | 1207 | NE | ARG | 155 | 2.733 | 22.696 | 84.054 | 1.00 | 74.79 |
| ATOM | 1208 | CZ | ARG | 155 | 3.518 | 23.385 | 83.228 | 1.00 | 75.80 |
| ATOM | 1209 | NH1 | ARG | 155 | 3.531 | 23.113 | 81.927 | 1.00 | 75.28 |
| ATOM | 1210 | NH2 | ARG | 155 | 4.299 | 24.347 | 83.702 | 1.00 | 76.38 |
| ATOM | 1211 | C | ARG | 155 | −1.288 | 20.178 | 80.074 | 1.00 | 65.87 |
| ATOM | 1212 | O | ARG | 155 | −1.017 | 20.366 | 78.895 | 1.00 | 65.37 |
| ATOM | 1213 | N | SER | 156 | −1.745 | 19.022 | 80.542 | 1.00 | 63.16 |
| ATOM | 1214 | CA | SER | 156 | −1.993 | 17.882 | 79.676 | 1.00 | 60.61 |
| ATOM | 1215 | CB | SER | 156 | −2.509 | 16.702 | 80.490 | 1.00 | 58.93 |
| ATOM | 1216 | OG | SER | 156 | −2.937 | 15.672 | 79.622 | 1.00 | 59.68 |
| ATOM | 1217 | C | SER | 156 | −3.008 | 18.224 | 78.591 | 1.00 | 59.40 |
| ATOM | 1218 | O | SER | 156 | −2.854 | 17.810 | 77.446 | 1.00 | 58.17 |
| ATOM | 1219 | N | GLN | 157 | −4.046 | 18.969 | 78.965 | 1.00 | 58.38 |
| ATOM | 1220 | CA | GLN | 157 | −5.077 | 19.364 | 78.021 | 1.00 | 58.88 |
| ATOM | 1221 | CB | GLN | 157 | −6.259 | 20.012 | 78.745 | 1.00 | 61.05 |
| ATOM | 1222 | CG | GLN | 157 | −7.047 | 19.058 | 79.649 | 1.00 | 64.28 |
| ATOM | 1223 | CD | GLN | 157 | −7.648 | 17.870 | 78.894 | 1.00 | 66.97 |
| ATOM | 1224 | OE1 | GLN | 157 | −6.932 | 17.108 | 78.228 | 1.00 | 67.14 |
| ATOM | 1225 | NE2 | GLN | 157 | −8.965 | 17.702 | 79.006 | 1.00 | 67.00 |
| ATOM | 1226 | C | GLN | 157 | −4.492 | 20.327 | 77.009 | 1.00 | 59.44 |
| ATOM | 1227 | O | GLN | 157 | −4.795 | 20.247 | 75.818 | 1.00 | 60.58 |
| ATOM | 1228 | N | ASP | 158 | −3.638 | 21.226 | 77.482 | 1.00 | 57.75 |
| ATOM | 1229 | CA | ASP | 158 | −2.992 | 22.194 | 76.611 | 1.00 | 57.99 |
| ATOM | 1230 | CB | ASP | 158 | −2.371 | 23.334 | 77.447 | 1.00 | 59.91 |
| ATOM | 1231 | CG | ASP | 158 | −3.427 | 24.269 | 78.068 | 1.00 | 63.30 |
| ATOM | 1232 | OD1 | ASP | 158 | −3.051 | 25.214 | 78.805 | 1.00 | 65.17 |
| ATOM | 1233 | OD2 | ASP | 158 | −4.635 | 24.070 | 77.815 | 1.00 | 64.66 |
| ATOM | 1234 | C | ASP | 158 | −1.925 | 21.543 | 75.703 | 1.00 | 56.75 |
| ATOM | 1235 | O | ASP | 158 | −1.823 | 21.902 | 74.525 | 1.00 | 53.72 |
| ATOM | 1236 | N | ASP | 159 | −1.140 | 20.600 | 76.241 | 1.00 | 55.82 |
| ATOM | 1237 | CA | ASP | 159 | −0.113 | 19.925 | 75.441 | 1.00 | 54.58 |
| ATOM | 1238 | CB | ASP | 159 | 0.695 | 18.888 | 76.250 | 1.00 | 58.46 |
| ATOM | 1239 | CG | ASP | 159 | 1.498 | 19.499 | 77.402 | 1.00 | 62.06 |
| ATOM | 1240 | OD1 | ASP | 159 | 2.156 | 20.552 | 77.223 | 1.00 | 63.94 |
| ATOM | 1241 | OD2 | ASP | 159 | 1.500 | 18.891 | 78.497 | 1.00 | 65.22 |
| ATOM | 1242 | C | ASP | 159 | −0.813 | 19.197 | 74.301 | 1.00 | 52.55 |
| ATOM | 1243 | O | ASP | 159 | −0.320 | 19.187 | 73.174 | 1.00 | 52.06 |
| ATOM | 1244 | N | VAL | 160 | −1.963 | 18.592 | 74.606 | 1.00 | 49.12 |
| ATOM | 1245 | CA | VAL | 160 | −2.759 | 17.870 | 73.603 | 1.00 | 49.39 |
| ATOM | 1246 | CB | VAL | 160 | −3.943 | 17.083 | 74.265 | 1.00 | 49.22 |
| ATOM | 1247 | CG1 | VAL | 160 | −4.896 | 16.550 | 73.202 | 1.00 | 48.51 |
| ATOM | 1248 | CG2 | VAL | 160 | −3.402 | 15.922 | 75.096 | 1.00 | 48.41 |
| ATOM | 1249 | C | VAL | 160 | −3.330 | 18.831 | 72.548 | 1.00 | 47.61 |
| ATOM | 1250 | O | VAL | 160 | −3.330 | 18.524 | 71.348 | 1.00 | 45.78 |
| ATOM | 1251 | N | GLN | 161 | −3.820 | 19.982 | 73.008 | 1.00 | 46.87 |
| ATOM | 1252 | CA | GLN | 161 | −4.385 | 21.007 | 72.121 | 1.00 | 47.61 |
| ATOM | 1253 | CB | GLN | 161 | −4.829 | 22.234 | 72.930 | 1.00 | 49.79 |
| ATOM | 1254 | CG | GLN | 161 | −5.289 | 23.402 | 72.059 | 1.00 | 48.02 |
| ATOM | 1255 | CD | GLN | 161 | −6.388 | 22.993 | 71.113 | 1.00 | 49.19 |
| ATOM | 1256 | OE1 | GLN | 161 | −7.441 | 22.526 | 71.540 | 1.00 | 49.82 |
| ATOM | 1257 | NE2 | GLN | 161 | −6.150 | 23.156 | 69.813 | 1.00 | 50.57 |
| ATOM | 1258 | C | GLN | 161 | −3.356 | 21.461 | 71.088 | 1.00 | 46.12 |
| ATOM | 1259 | O | GLN | 161 | −3.680 | 21.734 | 69.931 | 1.00 | 45.79 |
| ATOM | 1260 | N | LYS | 162 | −2.113 | 21.544 | 71.535 | 1.00 | 44.41 |
| ATOM | 1261 | CA | LYS | 162 | −1.026 | 21.969 | 70.687 | 1.00 | 44.22 |
| ATOM | 1262 | CB | LYS | 162 | 0.173 | 22.336 | 71.572 | 1.00 | 46.80 |
| ATOM | 1263 | CG | LYS | 162 | −0.151 | 23.474 | 72.548 | 1.00 | 48.45 |
| ATOM | 1264 | CD | LYS | 162 | 1.042 | 23.861 | 73.410 | 1.00 | 55.00 |
| ATOM | 1265 | CE | LYS | 162 | 0.782 | 25.121 | 74.240 | 1.00 | 55.12 |
| ATOM | 1266 | NZ | LYS | 162 | −0.428 | 24.984 | 75.112 | 1.00 | 58.91 |
| ATOM | 1267 | C | LYS | 162 | −0.696 | 20.868 | 69.681 | 1.00 | 42.73 |
| ATOM | 1268 | O | LYS | 162 | −0.371 | 21.148 | 68.538 | 1.00 | 44.92 |
| ATOM | 1269 | N | LEU | 163 | −0.784 | 19.613 | 70.110 | 1.00 | 41.72 |
| ATOM | 1270 | CA | LEU | 163 | −0.545 | 18.492 | 69.213 | 1.00 | 38.98 |
| ATOM | 1271 | CB | LEU | 163 | −0.664 | 17.170 | 69.962 | 1.00 | 36.52 |
| ATOM | 1272 | CG | LEU | 163 | 0.584 | 16.760 | 70.740 | 1.00 | 40.24 |
| ATOM | 1273 | CD1 | LEU | 163 | 0.308 | 15.468 | 71.517 | 1.00 | 39.87 |
| ATOM | 1274 | CD2 | LEU | 163 | 1.757 | 16.585 | 69.753 | 1.00 | 36.04 |
| ATOM | 1275 | C | LEU | 163 | −1.584 | 18.550 | 68.102 | 1.00 | 37.77 |
| ATOM | 1276 | O | LEU | 163 | −1.290 | 18.274 | 66.950 | 1.00 | 37.29 |
| ATOM | 1277 | N | THR | 164 | −2.808 | 18.907 | 68.476 | 1.00 | 38.49 |
| ATOM | 1278 | CA | THR | 164 | −3.914 | 19.038 | 67.538 | 1.00 | 37.80 |
| ATOM | 1279 | CB | THR | 164 | −5.209 | 19.367 | 68.298 | 1.00 | 38.14 |
| ATOM | 1280 | OG1 | THR | 164 | −5.405 | 18.386 | 69.321 | 1.00 | 39.84 |
| ATOM | 1281 | CG2 | THR | 164 | −6.412 | 19.353 | 67.366 | 1.00 | 36.51 |
| ATOM | 1282 | C | THR | 164 | −3.584 | 20.170 | 66.562 | 1.00 | 38.02 |
| ATOM | 1283 | O | THR | 164 | −3.753 | 20.016 | 65.356 | 1.00 | 38.15 |
| ATOM | 1284 | N | ASP | 165 | −3.097 | 21.290 | 67.100 | 1.00 | 38.34 |
| ATOM | 1285 | CA | ASP | 165 | −2.709 | 22.463 | 66.307 | 1.00 | 42.07 |
| ATOM | 1286 | CB | ASP | 165 | −2.118 | 23.573 | 67.205 | 1.00 | 45.79 |
| ATOM | 1287 | CG | ASP | 165 | −3.139 | 24.181 | 68.158 | 1.00 | 49.06 |
| ATOM | 1288 | OD1 | ASP | 165 | −2.716 | 24.949 | 69.057 | 1.00 | 51.38 |
| ATOM | 1289 | OD2 | ASP | 165 | −4.355 | 23.905 | 68.005 | 1.00 | 48.41 |
| ATOM | 1290 | C | ASP | 165 | −1.670 | 22.124 | 65.251 | 1.00 | 40.17 |
| ATOM | 1291 | O | ASP | 165 | −1.809 | 22.489 | 64.093 | 1.00 | 39.87 |
| ATOM | 1292 | N | ALA | 166 | −0.607 | 21.443 | 65.658 | 1.00 | 39.77 |
| ATOM | 1293 | CA | ALA | 166 | 0.430 | 21.079 | 64.699 | 1.00 | 38.11 |
| ATOM | 1294 | CB | ALA | 166 | 1.659 | 20.514 | 65.439 | 1.00 | 36.43 |
| ATOM | 1295 | C | ALA | 166 | −0.096 | 20.067 | 63.663 | 1.00 | 35.40 |
| ATOM | 1296 | O | ALA | 166 | 0.291 | 20.104 | 62.492 | 1.00 | 36.48 |
| ATOM | 1297 | N | ALA | 167 | −0.976 | 19.165 | 64.086 | 1.00 | 32.91 |
| ATOM | 1298 | CA | ALA | 167 | −1.506 | 18.188 | 63.148 | 1.00 | 32.56 |
| ATOM | 1299 | CB | ALA | 167 | −2.260 | 17.075 | 63.900 | 1.00 | 27.68 |
| ATOM | 1300 | C | ALA | 167 | −2.434 | 18.925 | 62.167 | 1.00 | 33.32 |
| ATOM | 1301 | O | ALA | 167 | −2.378 | 18.708 | 60.957 | 1.00 | 32.20 |
| ATOM | 1302 | N | ILE | 168 | −3.276 | 19.815 | 62.690 | 1.00 | 33.15 |
| ATOM | 1303 | CA | ILE | 168 | −4.173 | 20.549 | 61.817 | 1.00 | 34.96 |
| ATOM | 1304 | CB | ILE | 168 | −5.224 | 21.422 | 62.650 | 1.00 | 36.24 |
| ATOM | 1305 | CG2 | ILE | 168 | −5.269 | 22.847 | 62.144 | 1.00 | 33.31 |
| ATOM | 1306 | CG1 | ILE | 168 | −6.660 | 20.870 | 62.468 | 1.00 | 38.04 |
| ATOM | 1307 | CD1 | ILE | 168 | −6.924 | 19.486 | 62.974 | 1.00 | 35.50 |
| ATOM | 1308 | C | ILE | 168 | −3.318 | 21.409 | 60.882 | 1.00 | 34.13 |
| ATOM | 1309 | O | ILE | 168 | −3.619 | 21.546 | 59.707 | 1.00 | 32.89 |
| ATOM | 1310 | N | LYS | 169 | −2.221 | 21.957 | 61.389 | 1.00 | 37.28 |
| ATOM | 1311 | CA | LYS | 169 | −1.364 | 22.798 | 60.539 | 1.00 | 39.15 |
| ATOM | 1312 | CB | LYS | 169 | −0.261 | 23.461 | 61.365 | 1.00 | 43.88 |
| ATOM | 1313 | CG | LYS | 169 | 0.749 | 24.246 | 60.509 | 1.00 | 48.23 |
| ATOM | 1314 | CD | LYS | 169 | 1.813 | 24.917 | 61.371 | 1.00 | 52.07 |
| ATOM | 1315 | CE | LYS | 169 | 2.902 | 25.630 | 60.530 | 1.00 | 54.04 |
| ATOM | 1316 | NZ | LYS | 169 | 2.387 | 26.724 | 59.637 | 1.00 | 55.30 |
| ATOM | 1317 | C | LYS | 169 | −0.744 | 22.059 | 59.342 | 1.00 | 36.63 |
| ATOM | 1318 | O | LYS | 169 | −0.649 | 22.616 | 58.258 | 1.00 | 36.33 |
| ATOM | 1319 | N | LYS | 170 | −0.336 | 20.808 | 59.525 | 1.00 | 36.30 |
| ATOM | 1320 | CA | LYS | 170 | 0.248 | 20.042 | 58.412 | 1.00 | 35.20 |
| ATOM | 1321 | CB | LYS | 170 | 0.798 | 18.694 | 58.907 | 1.00 | 35.03 |
| ATOM | 1322 | CG | LYS | 170 | 2.026 | 18.786 | 59.780 | 1.00 | 35.34 |
| ATOM | 1323 | CD | LYS | 170 | 2.268 | 17.475 | 60.522 | 1.00 | 37.56 |
| ATOM | 1324 | CE | LYS | 170 | 3.459 | 17.597 | 61.468 | 1.00 | 43.01 |
| ATOM | 1325 | NZ | LYS | 170 | 3.540 | 16.490 | 62.471 | 1.00 | 43.46 |
| ATOM | 1326 | C | LYS | 170 | −0.797 | 19.788 | 57.322 | 1.00 | 34.52 |
| ATOM | 1327 | O | LYS | 170 | −0.503 | 19.889 | 56.140 | 1.00 | 36.16 |
| ATOM | 1328 | N | ILE | 171 | −2.013 | 19.439 | 57.734 | 1.00 | 34.26 |
| ATOM | 1329 | CA | ILE | 171 | −3.102 | 19.180 | 56.802 | 1.00 | 33.78 |
| ATOM | 1330 | CB | ILE | 171 | −4.433 | 18.788 | 57.543 | 1.00 | 31.23 |
| ATOM | 1331 | CG2 | ILE | 171 | −5.614 | 18.890 | 56.563 | 1.00 | 28.07 |
| ATOM | 1332 | CG1 | ILE | 171 | −4.312 | 17.386 | 58.162 | 1.00 | 26.76 |
| ATOM | 1333 | CD1 | ILE | 171 | −5.344 | 17.070 | 59.301 | 1.00 | 20.37 |
| ATOM | 1334 | C | ILE | 171 | −3.340 | 20.430 | 55.977 | 1.00 | 32.17 |
| ATOM | 1335 | O | ILE | 171 | −3.469 | 20.358 | 54.769 | 1.00 | 34.63 |
| ATOM | 1336 | N | GLU | 172 | −3.379 | 21.580 | 56.629 | 1.00 | 34.69 |
| ATOM | 1337 | CA | GLU | 172 | −3.606 | 22.839 | 55.918 | 1.00 | 35.82 |
| ATOM | 1338 | CB | GLU | 172 | −3.678 | 24.007 | 56.911 | 1.00 | 37.88 |
| ATOM | 1339 | CG | GLU | 172 | −4.713 | 23.810 | 58.011 | 1.00 | 43.69 |
| ATOM | 1340 | CD | GLU | 172 | −6.141 | 23.889 | 57.503 | 1.00 | 47.30 |
| ATOM | 1341 | OE1 | GLU | 172 | −6.339 | 23.631 | 56.293 | 1.00 | 49.76 |
| ATOM | 1342 | OE2 | GLU | 172 | −7.058 | 24.193 | 58.312 | 1.00 | 47.70 |
| ATOM | 1343 | C | GLU | 172 | −2.494 | 23.092 | 54.895 | 1.00 | 36.71 |
| ATOM | 1344 | O | GLU | 172 | −2.769 | 23.503 | 53.756 | 1.00 | 38.19 |
| ATOM | 1345 | N | ALA | 173 | −1.241 | 22.839 | 55.284 | 1.00 | 37.14 |
| ATOM | 1346 | CA | ALA | 173 | −0.103 | 23.053 | 54.367 | 1.00 | 37.45 |
| ATOM | 1347 | CB | ALA | 173 | 1.230 | 22.912 | 55.109 | 1.00 | 36.11 |
| ATOM | 1348 | C | ALA | 173 | −0.153 | 22.079 | 53.201 | 1.00 | 36.09 |
| ATOM | 1349 | O | ALA | 173 | 0.136 | 22.428 | 52.066 | 1.00 | 39.05 |
| ATOM | 1350 | N | ALA | 174 | −0.513 | 20.843 | 53.472 | 1.00 | 35.66 |
| ATOM | 1351 | CA | ALA | 174 | −0.598 | 19.905 | 52.375 | 1.00 | 36.41 |
| ATOM | 1352 | CB | ALA | 174 | −0.873 | 18.518 | 52.901 | 1.00 | 36.97 |
| ATOM | 1353 | C | ALA | 174 | −1.715 | 20.363 | 51.419 | 1.00 | 33.92 |
| ATOM | 1354 | O | ALA | 174 | −1.558 | 20.279 | 50.213 | 1.00 | 33.46 |
| ATOM | 1355 | N | LEU | 175 | −2.827 | 20.862 | 51.970 | 1.00 | 34.46 |
| ATOM | 1356 | CA | LEU | 175 | −3.958 | 21.331 | 51.162 | 1.00 | 33.61 |
| ATOM | 1357 | CB | LEU | 175 | −5.154 | 21.716 | 52.058 | 1.00 | 34.33 |
| ATOM | 1358 | CG | LEU | 175 | −6.356 | 22.318 | 51.296 | 1.00 | 32.56 |
| ATOM | 1359 | CD1 | LEU | 175 | −6.771 | 21.397 | 50.123 | 1.00 | 28.31 |
| ATOM | 1360 | CD2 | LEU | 175 | −7.512 | 22.540 | 52.259 | 1.00 | 29.36 |
| ATOM | 1361 | C | LEU | 175 | −3.612 | 22.518 | 50.263 | 1.00 | 33.20 |
| ATOM | 1362 | O | LEU | 175 | −3.888 | 22.503 | 49.064 | 1.00 | 31.43 |
| ATOM | 1363 | N | ALA | 176 | −3.012 | 23.542 | 50.863 | 1.00 | 34.82 |
| ATOM | 1364 | CA | ALA | 176 | −2.616 | 24.746 | 50.146 | 1.00 | 34.42 |
| ATOM | 1365 | CB | ALA | 176 | −1.953 | 25.712 | 51.106 | 1.00 | 35.13 |
| ATOM | 1366 | C | ALA | 176 | −1.668 | 24.418 | 48.989 | 1.00 | 37.83 |
| ATOM | 1367 | O | ALA | 176 | −1.868 | 24.882 | 47.866 | 1.00 | 38.78 |
| ATOM | 1368 | N | ASP | 177 | −0.630 | 23.627 | 49.248 | 1.00 | 39.20 |
| ATOM | 1369 | CA | ASP | 177 | 0.281 | 23.279 | 48.171 | 1.00 | 41.20 |
| ATOM | 1370 | CB | ASP | 177 | 1.397 | 22.365 | 48.664 | 1.00 | 45.40 |
| ATOM | 1371 | CG | ASP | 177 | 2.318 | 23.065 | 49.640 | 1.00 | 53.11 |
| ATOM | 1372 | OD1 | ASP | 177 | 2.689 | 24.232 | 49.361 | 1.00 | 54.79 |
| ATOM | 1373 | OD2 | ASP | 177 | 2.687 | 22.457 | 50.674 | 1.00 | 55.87 |
| ATOM | 1374 | C | ASP | 177 | −0.473 | 22.597 | 47.047 | 1.00 | 41.24 |
| ATOM | 1375 | O | ASP | 177 | −0.196 | 22.834 | 45.872 | 1.00 | 39.86 |
| ATOM | 1376 | N | LYS | 178 | −1.433 | 21.749 | 47.408 | 1.00 | 41.20 |
| ATOM | 1377 | CA | LYS | 178 | −2.226 | 21.049 | 46.402 | 1.00 | 42.30 |
| ATOM | 1378 | CB | LYS | 178 | −3.077 | 19.959 | 47.065 | 1.00 | 44.35 |
| ATOM | 1379 | CG | LYS | 178 | −3.957 | 19.180 | 46.096 | 1.00 | 44.13 |
| ATOM | 1380 | CD | LYS | 178 | −3.153 | 18.478 | 45.013 | 1.00 | 41.10 |
| ATOM | 1381 | CE | LYS | 178 | −2.255 | 17.422 | 45.586 | 1.00 | 39.67 |
| ATOM | 1382 | NZ | LYS | 178 | −1.734 | 16.546 | 44.503 | 1.00 | 41.20 |
| ATOM | 1383 | C | LYS | 178 | −3.105 | 22.011 | 45.584 | 1.00 | 41.77 |
| ATOM | 1384 | O | LYS | 178 | −3.145 | 21.916 | 44.365 | 1.00 | 43.66 |
| ATOM | 1385 | N | GLU | 179 | −3.802 | 22.942 | 46.227 | 1.00 | 41.77 |
| ATOM | 1386 | CA | GLU | 179 | −4.601 | 23.892 | 45.453 | 1.00 | 43.66 |
| ATOM | 1387 | CB | GLU | 179 | −5.490 | 24.756 | 46.351 | 1.00 | 43.35 |
| ATOM | 1388 | CG | GLU | 179 | −6.538 | 23.967 | 47.098 | 1.00 | 47.58 |
| ATOM | 1389 | CD | GLU | 179 | −7.658 | 24.835 | 47.654 | 1.00 | 49.87 |
| ATOM | 1390 | OE1 | GLU | 179 | −8.292 | 25.571 | 46.855 | 1.00 | 51.89 |
| ATOM | 1391 | OE2 | GLU | 179 | −7.916 | 24.775 | 48.880 | 1.00 | 48.80 |
| ATOM | 1392 | C | GLU | 179 | −3.685 | 24.797 | 44.629 | 1.00 | 44.17 |
| ATOM | 1393 | O | GLU | 179 | −4.041 | 25.192 | 43.526 | 1.00 | 46.42 |
| ATOM | 1394 | N | ALA | 180 | −2.511 | 25.132 | 45.163 | 1.00 | 44.61 |
| ATOM | 1395 | CA | ALA | 180 | −1.559 | 25.979 | 44.438 | 1.00 | 45.76 |
| ATOM | 1396 | CB | ALA | 180 | −0.359 | 26.289 | 45.300 | 1.00 | 43.35 |
| ATOM | 1397 | C | ALA | 180 | −1.129 | 25.217 | 43.197 | 1.00 | 47.11 |
| ATOM | 1398 | O | ALA | 180 | −0.914 | 25.792 | 42.133 | 1.00 | 48.59 |
| ATOM | 1399 | N | GLU | 181 | −1.028 | 23.903 | 43.352 | 1.00 | 47.73 |
| ATOM | 1400 | CA | GLU | 181 | −0.651 | 23.002 | 42.277 | 1.00 | 48.37 |
| ATOM | 1401 | CB | GLU | 181 | −0.452 | 21.600 | 42.874 | 1.00 | 50.12 |
| ATOM | 1402 | CG | GLU | 181 | 0.477 | 20.663 | 42.114 | 1.00 | 50.74 |
| ATOM | 1403 | CD | GLU | 181 | 0.533 | 19.276 | 42.751 | 1.00 | 51.08 |
| ATOM | 1404 | OE1 | GLU | 181 | −0.476 | 18.535 | 42.693 | 1.00 | 50.45 |
| ATOM | 1405 | OE2 | GLU | 181 | 1.584 | 18.933 | 43.324 | 1.00 | 49.78 |
| ATOM | 1406 | C | GLU | 181 | −1.792 | 23.000 | 41.238 | 1.00 | 49.22 |
| ATOM | 1407 | O | GLU | 181 | −1.559 | 23.210 | 40.058 | 1.00 | 48.41 |
| ATOM | 1408 | N | LEU | 182 | −3.021 | 22.754 | 41.696 | 1.00 | 50.42 |
| ATOM | 1409 | CA | LEU | 182 | −4.208 | 22.735 | 40.832 | 1.00 | 51.74 |
| ATOM | 1410 | CB | LEU | 182 | −5.486 | 22.494 | 41.651 | 1.00 | 49.82 |
| ATOM | 1411 | CG | LEU | 182 | −5.941 | 21.106 | 42.089 | 1.00 | 50.56 |
| ATOM | 1412 | CD1 | LEU | 182 | −6.212 | 20.264 | 40.855 | 1.00 | 49.40 |
| ATOM | 1413 | CD2 | LEU | 182 | −4.901 | 20.464 | 42.954 | 1.00 | 48.77 |
| ATOM | 1414 | C | LEU | 182 | −4.411 | 24.036 | 40.072 | 1.00 | 54.20 |
| ATOM | 1415 | O | LEU | 182 | −4.818 | 24.041 | 38.910 | 1.00 | 53.28 |
| ATOM | 1416 | N | MET | 183 | −4.149 | 25.134 | 40.768 | 1.00 | 56.78 |
| ATOM | 1417 | CA | MET | 183 | −4.308 | 26.475 | 40.245 | 1.00 | 60.66 |
| ATOM | 1418 | CB | MET | 183 | −4.554 | 27.413 | 41.418 | 1.00 | 61.27 |
| ATOM | 1419 | CG | MET | 183 | −4.745 | 28.857 | 41.067 | 1.00 | 63.93 |
| ATOM | 1420 | SD | MET | 183 | −4.901 | 29.814 | 42.586 | 1.00 | 68.53 |
| ATOM | 1421 | CE | MET | 183 | −6.330 | 28.989 | 43.348 | 1.00 | 67.90 |
| ATOM | 1422 | C | MET | 183 | −3.071 | 26.916 | 39.482 | 1.00 | 64.03 |
| ATOM | 1423 | O | MET | 183 | −3.052 | 27.987 | 38.869 | 1.00 | 64.46 |
| ATOM | 1424 | N | GLN | 184 | −2.042 | 26.076 | 39.514 | 1.00 | 67.44 |
| ATOM | 1425 | CA | GLN | 184 | −0.777 | 26.383 | 38.860 | 1.00 | 71.23 |
| ATOM | 1426 | CB | GLN | 184 | 0.222 | 25.242 | 39.116 | 1.00 | 73.44 |
| ATOM | 1427 | CG | GLN | 184 | 1.697 | 25.659 | 39.291 | 1.00 | 76.77 |
| ATOM | 1428 | CD | GLN | 184 | 2.348 | 26.207 | 38.020 | 1.00 | 79.02 |
| ATOM | 1429 | OE1 | GLN | 184 | 1.893 | 27.204 | 37.442 | 1.00 | 79.20 |
| ATOM | 1430 | NE2 | GLN | 184 | 3.428 | 25.554 | 37.586 | 1.00 | 79.55 |
| ATOM | 1431 | C | GLN | 184 | −0.985 | 26.598 | 37.360 | 1.00 | 72.64 |
| ATOM | 1432 | O | GLN | 184 | −0.971 | 27.737 | 36.873 | 1.00 | 72.31 |
| ATOM | 1433 | N | ALA | 185 | −1.189 | 25.507 | 36.633 | 1.00 | 74.64 |
| ATOM | 1434 | CA | ALA | 185 | −1.387 | 25.593 | 35.195 | 1.00 | 76.72 |
| ATOM | 1435 | CB | ALA | 185 | −0.351 | 24.721 | 34.473 | 1.00 | 76.74 |
| ATOM | 1436 | C | ALA | 185 | −2.800 | 25.169 | 34.812 | 1.00 | 77.63 |
| ATOM | 1437 | O | ALA | 185 | −3.510 | 25.901 | 34.116 | 1.00 | 79.25 |
| ATOM | 1438 | O1 | HOH | 302 | 5.786 | 24.929 | 33.706 | 1.00 | 46.39 |
| ATOM | 1439 | O1 | HOH | 303 | 16.711 | 7.421 | 13.716 | 1.00 | 40.03 |
| ATOM | 1440 | O1 | HOH | 304 | −0.640 | 6.780 | 57.814 | 1.00 | 49.80 |
| ATOM | 1441 | O1 | HOH | 305 | −3.664 | 8.728 | 22.988 | 1.00 | 37.04 |
| ATOM | 1442 | O1 | HOH | 307 | −6.985 | 9.376 | 71.599 | 1.00 | 44.76 |
| ATOM | 1443 | O1 | HOH | 309 | 2.439 | 23.127 | 69.818 | 1.00 | 64.96 |
| ATOM | 1444 | O1 | HOH | 310 | −12.357 | 22.793 | 59.997 | 1.00 | 49.37 |
| ATOM | 1445 | O1 | HOH | 311 | −5.000 | 9.881 | 15.800 | 1.00 | 39.08 |
| ATOM | 1446 | O1 | HOH | 312 | −1.379 | 2.409 | 21.615 | 1.00 | 37.20 |
| ATOM | 1447 | O1 | HOH | 314 | 4.226 | 11.609 | 56.051 | 1.00 | 61.48 |
| ATOM | 1448 | O1 | HOH | 315 | 3.062 | 22.032 | 74.337 | 1.00 | 57.32 |
| ATOM | 1449 | O1 | HOH | 316 | 2.291 | 19.201 | 55.609 | 1.00 | 32.55 |
| ATOM | 1450 | O1 | HOH | 317 | −14.260 | 15.606 | 65.187 | 1.00 | 48.61 |
| ATOM | 1451 | O1 | HOH | 318 | 1.372 | 26.408 | 49.218 | 1.00 | 45.06 |
| ATOM | 1452 | O1 | HOH | 320 | −6.791 | 23.072 | 66.093 | 1.00 | 55.37 |
| ATOM | 1453 | O1 | HOH | 321 | −5.960 | 5.653 | 7.525 | 1.00 | 57.42 |
| ATOM | 1454 | O1 | HOH | 322 | 9.023 | 7.824 | 4.729 | 1.00 | 49.88 |
| ATOM | 1455 | O1 | HOH | 323 | −5.164 | 25.011 | 53.787 | 1.00 | 48.29 |
| ATOM | 1456 | O1 | HOH | 324 | −7.620 | 15.194 | 35.505 | 1.00 | 42.96 |
| ATOM | 1457 | O1 | HOH | 325 | 0.761 | 16.491 | 65.949 | 1.00 | 49.53 |
| ATOM | 1458 | O1 | HOH | 327 | −13.358 | 14.152 | 48.865 | 1.00 | 49.48 |
| ATOM | 1459 | O1 | HOH | 328 | 9.574 | 23.698 | 19.099 | 1.00 | 70.52 |
| ATOM | 1460 | O1 | HOH | 329 | −15.006 | 12.044 | 46.069 | 1.00 | 61.30 |
| ATOM | 1461 | O1 | HOH | 330 | 14.950 | 11.135 | 27.309 | 1.00 | 82.65 |
| ATOM | 1462 | O1 | HOH | 331 | −6.107 | 10.675 | 18.376 | 1.00 | 43.27 |
| ATOM | 1463 | O1 | HOH | 332 | −17.369 | 21.115 | 39.828 | 1.00 | 59.43 |
| ATOM | 1464 | O1 | HOH | 333 | 3.753 | 16.179 | 57.494 | 1.00 | 71.35 |
| ATOM | 1465 | O1 | HOH | 334 | −15.680 | 10.875 | 74.742 | 1.00 | 61.46 |
| ATOM | 1466 | O1 | HOH | 335 | −15.080 | 23.421 | 48.843 | 1.00 | 55.54 |
| ATOM | 1467 | O1 | HOH | 336 | −17.698 | 17.552 | 36.236 | 1.00 | 49.63 |
| ATOM | 1468 | O1 | HOH | 337 | −3.687 | 11.077 | 13.706 | 1.00 | 31.62 |
| ATOM | 1469 | O1 | HOH | 338 | 3.927 | 6.239 | 5.123 | 1.00 | 44.26 |
| ATOM | 1470 | O1 | HOH | 339 | 2.369 | 24.751 | 52.035 | 1.00 | 50.57 |
| ATOM | 1471 | O1 | HOH | 340 | −15.269 | 15.498 | 54.910 | 1.00 | 75.04 |
| ATOM | 1472 | O1 | HOH | 341 | 15.978 | 10.746 | 6.920 | 1.00 | 59.72 |
| ATOM | 1473 | O1 | HOH | 342 | −4.143 | 18.035 | 17.666 | 1.00 | 41.15 |
| ATOM | 1474 | O1 | HOH | 343 | −14.940 | 13.136 | 37.292 | 1.00 | 58.44 |
| ATOM | 1475 | O1 | HOH | 344 | 2.563 | 12.714 | 60.213 | 1.00 | 57.07 |
| ATOM | 1476 | O1 | HOH | 346 | 1.596 | 13.582 | 86.916 | 1.00 | 70.91 |
| ATOM | 1477 | O1 | HOH | 347 | 7.838 | 16.595 | 73.384 | 1.00 | 59.18 |
| ATOM | 1478 | O1 | HOH | 348 | −6.147 | 9.577 | 11.401 | 1.00 | 57.10 |
| ATOM | 1479 | O1 | HOH | 349 | −14.722 | 27.444 | 46.158 | 1.00 | 90.16 |
| ATOM | 1480 | O1 | HOH | 350 | −4.140 | 13.336 | 31.273 | 1.00 | 33.25 |
| ATOM | 1481 | O1 | HOH | 351 | −2.060 | 25.041 | 85.618 | 1.00 | 80.45 |
| ATOM | 1482 | O1 | HOH | 352 | −11.312 | 11.026 | 39.981 | 1.00 | 45.94 |
| ATOM | 1483 | O1 | HOH | 353 | −17.003 | 20.181 | 54.859 | 1.00 | 71.86 |
| ATOM | 1484 | O1 | HOH | 354 | −0.916 | 3.322 | 71.864 | 1.00 | 49.68 |
| ATOM | 1485 | O1 | HOH | 355 | −7.005 | 20.501 | 13.831 | 1.00 | 53.66 |
| ATOM | 1486 | O1 | HOH | 356 | −14.600 | 23.546 | 55.631 | 1.00 | 48.35 |
| ATOM | 1487 | O1 | HOH | 357 | −17.819 | 23.151 | 50.976 | 1.00 | 52.11 |
| ATOM | 1488 | O1 | HOH | 358 | 2.254 | 26.577 | 21.628 | 1.00 | 60.67 |
| ATOM | 1489 | O1 | HOH | 359 | 0.803 | 16.354 | 50.390 | 1.00 | 52.48 |
| ATOM | 1490 | O1 | HOH | 360 | 17.200 | 10.484 | 22.120 | 1.00 | 51.01 |
| ATOM | 1491 | O1 | HOH | 361 | −15.864 | 16.596 | 42.676 | 1.00 | 56.63 |
| ATOM | 1492 | O1 | HOH | 362 | 2.181 | 8.776 | 24.952 | 1.00 | 38.73 |
| ATOM | 1493 | O1 | HOH | 363 | −7.968 | 23.240 | 28.701 | 1.00 | 50.39 |
| ATOM | 1494 | O1 | HOH | 364 | −4.004 | 0.134 | 10.731 | 1.00 | 90.56 |
| ATOM | 1495 | O1 | HOH | 365 | −3.704 | 10.675 | 31.678 | 1.00 | 30.70 |
| ATOM | 1496 | O1 | HOH | 366 | −13.266 | 25.262 | 52.064 | 1.00 | 62.28 |
| ATOM | 1497 | O1 | HOH | 367 | −16.916 | 18.510 | 59.343 | 1.00 | 47.69 |
| ATOM | 1498 | O1 | HOH | 368 | −3.703 | 25.280 | 60.980 | 1.00 | 53.12 |
| ATOM | 1499 | O1 | HOH | 369 | −0.275 | 16.417 | 37.735 | 1.00 | 66.01 |
| ATOM | 1500 | O1 | HOH | 370 | −7.122 | 20.168 | 74.323 | 1.00 | 40.70 |
| ATOM | 1501 | O1 | HOH | 371 | 4.079 | 24.812 | 54.526 | 1.00 | 57.35 |
| ATOM | 1502 | O1 | HOH | 373 | −9.613 | 19.800 | 80.552 | 1.00 | 64.39 |
| ATOM | 1503 | O1 | HOH | 374 | −9.716 | 25.634 | 31.190 | 1.00 | 50.17 |
| ATOM | 1504 | O1 | HOH | 375 | −0.532 | 22.524 | 36.466 | 1.00 | 61.07 |
| ATOM | 1505 | O1 | HOH | 376 | −14.409 | 13.165 | 83.189 | 1.00 | 63.05 |
| ATOM | 1506 | O1 | HOH | 377 | −11.018 | 21.644 | 53.328 | 1.00 | 44.58 |
| ATOM | 1507 | O1 | HOH | 378 | 5.370 | 26.188 | 22.596 | 1.00 | 59.67 |
| ATOM | 1508 | O1 | HOH | 380 | −13.995 | 17.778 | 73.426 | 1.00 | 79.09 |
| ATOM | 1509 | O1 | HOH | 381 | 4.885 | −0.329 | 8.120 | 1.00 | 74.85 |
| ATOM | 1510 | O1 | HOH | 382 | 1.331 | 11.313 | 6.097 | 1.00 | 47.98 |
| ATOM | 1511 | O1 | HOH | 383 | 11.281 | 12.141 | 6.394 | 1.00 | 56.27 |
| ATOM | 1512 | O1 | HOH | 400 | −13.284 | 11.946 | 58.880 | 1.00 | 47.79 |
| ATOM | 1513 | O1 | HOH | 402 | −15.563 | 20.103 | 49.349 | 1.00 | 51.23 |
| ATOM | 1514 | O1 | HOH | 403 | 2.253 | 19.239 | 4.410 | 1.00 | 73.44 |
| ATOM | 1515 | O1 | HOH | 405 | 11.304 | 2.402 | 13.475 | 1.00 | 84.55 |
| ATOM | 1516 | O1 | HOH | 406 | −0.935 | 13.768 | 34.803 | 1.00 | 48.34 |
| ATOM | 1517 | O1 | HOH | 407 | −3.810 | 21.771 | 34.390 | 1.00 | 43.81 |
| ATOM | 1518 | O1 | HOH | 408 | −3.265 | 3.486 | 47.748 | 1.00 | 66.97 |
| ATOM | 1519 | O1 | HOH | 409 | 1.104 | 9.945 | 9.880 | 1.00 | 49.13 |
| ATOM | 1520 | O1 | HOH | 410 | 0.422 | 16.427 | 34.414 | 1.00 | 87.71 |
| ATOM | 1521 | O1 | HOH | 411 | −2.673 | −0.494 | 19.885 | 1.00 | 52.04 |
| ATOM | 1522 | O1 | HOH | 412 | −3.089 | 13.895 | 92.403 | 1.00 | 59.96 |
| ATOM | 1523 | O1 | HOH | 413 | 3.194 | 24.668 | 20.476 | 1.00 | 64.45 |
| ATOM | 1524 | O1 | HOH | 415 | −8.097 | 12.619 | 54.223 | 1.00 | 40.52 |
| ATOM | 1525 | O1 | HOH | 416 | 2.795 | 7.151 | 22.607 | 1.00 | 54.61 |
| ATOM | 1526 | O1 | HOH | 417 | 3.751 | 5.165 | 25.893 | 1.00 | 44.77 |
| ATOM | 1527 | O1 | HOH | 418 | 1.622 | 9.842 | 42.243 | 1.00 | 56.21 |
| ATOM | 1528 | O1 | HOH | 419 | 0.421 | 23.884 | 77.749 | 1.00 | 61.98 |
| ATOM | 1529 | O1 | HOH | 420 | 14.627 | 6.089 | 21.772 | 1.00 | 57.19 |
| ATOM | 1530 | O1 | HOH | 421 | 13.038 | 12.816 | 22.645 | 1.00 | 64.28 |
| ATOM | 1531 | O1 | HOH | 422 | −11.444 | 26.483 | 43.160 | 1.00 | 58.77 |
| ATOM | 1532 | O1 | HOH | 423 | 4.279 | 12.562 | 88.202 | 1.00 | 62.72 |
| ATOM | 1533 | O1 | HOH | 424 | −12.414 | 14.580 | 87.763 | 1.00 | 89.26 |
| ATOM | 1534 | O1 | HOH | 425 | −16.766 | 14.530 | 45.869 | 1.00 | 96.42 |
| ATOM | 1535 | O1 | HOH | 426 | 7.867 | 11.318 | 7.942 | 1.00 | 55.18 |
| ATOM | 1536 | O1 | HOH | 427 | 4.366 | 8.492 | 6.798 | 1.00 | 70.18 |
| ATOM | 1537 | O1 | HOH | 428 | −2.106 | 3.432 | 77.575 | 1.00 | 57.89 |
| ATOM | 1538 | O1 | HOH | 429 | 18.086 | 17.462 | 19.670 | 1.00 | 70.05 |
| ATOM | 1539 | O1 | HOH | 430 | −6.737 | 8.016 | 94.166 | 1.00 | 59.01 |
| ATOM | 1540 | O1 | HOH | 431 | 6.971 | 9.475 | 58.674 | 1.00 | 80.86 |
| ATOM | 1541 | O1 | HOH | 432 | −1.536 | 19.816 | 31.738 | 1.00 | 49.54 |
| ATOM | 1542 | O1 | HOH | 433 | 7.698 | 10.437 | 31.906 | 1.00 | 61.73 |
| ATOM | 1543 | O1 | HOH | 434 | 0.207 | 20.512 | 7.972 | 1.00 | 74.94 |
| ATOM | 1544 | O1 | HOH | 435 | 3.776 | 13.746 | 6.429 | 1.00 | 75.18 |
| ATOM | 1545 | O1 | HOH | 436 | −18.571 | 19.732 | 52.567 | 1.00 | 63.53 |
| ATOM | 1546 | O1 | HOH | 437 | −1.899 | 29.425 | 52.295 | 1.00 | 55.12 |
| ATOM | 1547 | O1 | HOH | 438 | −6.880 | 6.890 | 17.770 | 1.00 | 49.51 |
| ATOM | 1548 | O1 | HOH | 439 | −6.912 | 14.453 | 37.516 | 1.00 | 51.30 |
| ATOM | 1549 | O1 | HOH | 440 | 9.467 | 16.541 | 12.773 | 1.00 | 54.26 |
| ATOM | 1550 | O1 | HOH | 441 | 2.279 | 17.814 | 53.244 | 1.00 | 61.77 |
| ATOM | 1551 | O1 | HOH | 442 | −2.650 | −0.463 | 13.822 | 1.00 | 46.01 |
| ATOM | 1552 | O1 | HOH | 443 | −3.215 | 24.966 | 74.028 | 1.00 | 62.02 |
| ATOM | 1553 | O1 | HOH | 444 | −4.683 | 14.010 | 33.817 | 1.00 | 65.38 |
| ATOM | 1554 | O1 | HOH | 445 | −8.336 | 27.942 | 89.666 | 1.00 | 52.95 |
| ATOM | 1555 | O1 | HOH | 446 | −4.502 | 12.079 | 81.403 | 1.00 | 90.88 |
| ATOM | 1556 | O1 | HOH | 447 | −9.457 | 24.981 | 58.998 | 1.00 | 52.30 |
| ATOM | 1557 | O1 | HOH | 448 | −9.756 | 25.777 | 88.998 | 1.00 | 62.22 |
| ATOM | 1558 | O1 | HOH | 449 | 0.421 | 18.268 | 48.931 | 1.00 | 46.66 |
| ATOM | 1559 | O1 | HOH | 450 | 2.933 | 26.992 | 56.334 | 1.00 | 74.71 |
| ATOM | 1560 | O1 | HOH | 451 | 0.759 | 26.113 | 64.001 | 1.00 | 59.06 |
| ATOM | 1561 | O1 | HOH | 452 | 1.084 | 6.151 | 31.450 | 1.00 | 56.68 |
| ATOM | 1562 | O1 | HOH | 453 | 5.884 | 4.519 | 29.654 | 1.00 | 56.39 |
| ATOM | 1563 | O1 | HOH | 454 | −15.425 | 18.155 | 64.235 | 1.00 | 72.96 |
| ATOM | 1564 | O1 | HOH | 455 | −0.171 | 13.254 | 39.710 | 1.00 | 74.60 |
| ATOM | 1565 | O1 | HOH | 456 | 14.866 | 20.258 | 26.412 | 1.00 | 46.29 |
| ATOM | 1566 | O1 | HOH | 457 | −14.275 | 3.131 | 87.758 | 1.00 | 59.31 |
| ATOM | 1567 | O1 | HOH | 458 | −4.332 | 5.479 | 80.377 | 1.00 | 79.39 |
| ATOM | 1568 | O1 | HOH | 459 | −2.873 | 25.788 | 71.842 | 1.00 | 79.58 |
| ATOM | 1569 | O1 | HOH | 460 | −18.438 | 26.604 | 40.069 | 1.00 | 61.56 |
| ATOM | 1570 | O1 | HOH | 461 | −17.600 | 14.799 | 67.139 | 1.00 | 57.68 |
| ATOM | 1571 | O1 | HOH | 462 | 18.682 | 10.751 | 18.572 | 1.00 | 52.48 |
| ATOM | 1572 | O1 | HOH | 463 | −1.155 | 16.670 | 9.382 | 1.00 | 52.73 |
| ATOM | 1573 | O1 | HOH | 464 | −7.994 | 4.789 | 73.142 | 1.00 | 58.99 |
| ATOM | 1574 | O1 | HOH | 465 | 2.878 | 15.527 | 89.468 | 1.00 | 53.82 |
| ATOM | 1575 | O1 | HOH | 466 | −14.384 | 15.627 | 85.915 | 1.00 | 59.37 |
| ATOM | 1576 | O1 | HOH | 467 | 2.898 | 22.812 | 45.093 | 1.00 | 59.10 |
| ATOM | 1577 | O1 | HOH | 468 | −0.751 | 11.409 | 92.643 | 1.00 | 70.59 |
| ATOM | 1578 | O1 | HOH | 469 | 5.961 | 11.789 | 58.672 | 1.00 | 63.17 |
| ATOM | 1579 | O1 | HOH | 470 | 17.813 | 12.012 | 26.528 | 1.00 | 46.16 |
| ATOM | 1580 | O1 | HOH | 471 | −12.149 | 25.188 | 31.130 | 1.00 | 55.19 |
| ATOM | 1581 | O1 | HOH | 472 | −15.854 | 29.561 | 37.766 | 1.00 | 78.50 |
| ATOM | 1582 | O1 | HOH | 473 | 0.237 | 23.917 | 15.562 | 1.00 | 69.78 |
| ATOM | 1583 | O1 | HOH | 474 | 1.289 | 4.512 | 6.743 | 1.00 | 81.85 |
| ATOM | 1584 | O1 | HOH | 476 | −8.588 | 25.220 | 33.408 | 1.00 | 70.20 |
| ATOM | 1585 | O1 | HOH | 477 | 6.612 | 5.903 | 2.424 | 1.00 | 69.70 |
| ATOM | 1586 | O1 | HOH | 478 | −11.595 | 11.694 | 52.934 | 1.00 | 67.26 |
| ATOM | 1587 | O1 | HOH | 479 | −6.127 | 9.039 | 42.656 | 1.00 | 74.38 |
| ATOM | 1588 | O1 | HOH | 480 | 4.050 | 28.730 | 37.477 | 1.00 | 74.96 |
| ATOM | 1589 | O1 | HOH | 481 | 8.643 | 22.280 | 26.068 | 1.00 | 66.23 |
| ATOM | 1590 | O1 | HOH | 482 | 5.057 | 18.842 | 63.792 | 1.00 | 84.51 |
| ATOM | 1591 | O1 | HOH | 483 | 11.232 | 20.655 | 13.298 | 1.00 | 78.39 |
| ATOM | 1592 | O1 | HOH | 484 | 8.429 | 7.903 | 29.673 | 1.00 | 63.19 |
| ATOM | 1593 | O1 | HOH | 485 | 2.108 | 19.893 | 73.346 | 1.00 | 55.11 |
| ATOM | 1594 | O1 | HOH | 486 | −6.372 | 25.790 | 68.263 | 1.00 | 65.80 |
| ATOM | 1595 | O1 | HOH | 487 | −13.205 | 18.613 | 68.124 | 1.00 | 86.13 |
| ATOM | 1596 | O1 | HOH | 488 | −8.286 | 11.850 | 17.825 | 1.00 | 45.69 |
| ATOM | 1597 | O1 | HOH | 489 | 7.059 | 5.906 | 5.738 | 1.00 | 58.55 |
| ATOM | 1598 | O1 | HOH | 490 | 2.959 | 27.083 | 27.258 | 1.00 | 52.49 |
| ATOM | 1599 | O1 | HOH | 491 | 2.930 | 19.627 | 39.466 | 1.00 | 56.92 |
| ATOM | 1600 | O1 | HOH | 492 | 13.904 | 13.945 | 10.116 | 1.00 | 53.45 |
| ATOM | 1601 | O1 | HOH | 493 | 6.999 | 6.052 | 26.735 | 1.00 | 66.18 |
| ATOM | 1602 | O1 | HOH | 494 | 18.877 | 11.256 | 9.403 | 1.00 | 62.85 |
| ATOM | 1603 | O1 | HOH | 495 | −14.299 | 15.165 | 36.983 | 1.00 | 54.65 |
| ATOM | 1604 | O1 | HOH | 496 | −2.145 | 20.024 | 90.307 | 1.00 | 58.77 |
| ATOM | 1605 | O1 | HOH | 497 | 12.960 | 14.184 | 7.677 | 1.00 | 44.90 |
| ATOM | 1606 | O1 | HOH | 498 | −0.577 | 22.082 | 22.522 | 1.00 | 84.17 |
| ATOM | 1607 | O1 | HOH | 499 | −8.915 | 23.163 | 54.891 | 1.00 | 47.87 |
| ATOM | 1608 | O1 | HOH | 500 | −5.904 | 19.990 | 16.699 | 1.00 | 68.72 |
| ATOM | 1609 | O1 | HOH | 501 | 7.604 | 22.425 | 14.079 | 1.00 | 83.85 |
| ATOM | 1610 | O1 | HOH | 502 | −5.745 | 23.331 | 22.836 | 1.00 | 71.67 |
| ATOM | 1611 | O1 | HOH | 503 | 12.713 | 6.777 | 5.150 | 1.00 | 68.39 |
| ATOM | 1612 | O1 | HOH | 504 | −8.006 | 21.972 | 84.763 | 1.00 | 67.50 |
| ATOM | 1613 | O1 | HOH | 505 | −6.453 | 17.333 | 12.456 | 1.00 | 75.63 |
| ATOM | 1614 | O1 | HOH | 506 | −3.191 | 6.789 | 49.810 | 1.00 | 63.94 |
| ATOM | 1615 | O1 | HOH | 507 | 4.446 | 24.626 | 63.212 | 1.00 | 85.62 |
| ATOM | 1616 | O1 | HOH | 508 | 10.825 | 10.927 | 25.007 | 1.00 | 52.28 |
| ATOM | 1617 | O1 | HOH | 509 | 1.751 | 28.971 | 44.260 | 1.00 | 88.85 |
| ATOM | 1618 | O1 | HOH | 510 | 5.304 | 11.175 | 65.843 | 1.00 | 86.55 |
| ATOM | 1619 | O1 | HOH | 511 | 3.016 | 25.072 | 34.499 | 1.00 | 56.55 |
| ATOM | 1620 | O1 | HOH | 512 | −7.777 | 21.602 | 36.643 | 1.00 | 88.35 |
| ATOM | 1621 | O1 | HOH | 513 | 11.033 | 20.553 | 20.646 | 1.00 | 42.22 |
| ATOM | 1622 | O1 | HOH | 514 | 1.225 | 4.220 | 72.811 | 1.00 | 68.80 |
| ATOM | 1623 | O1 | HOH | 600 | −6.830 | 12.211 | 91.855 | 1.00 | 60.53 |
| ATOM | 1625 | O1 | HOH | 602 | −6.789 | 6.449 | 80.935 | 1.00 | 50.26 |
| ATOM | 1626 | O1 | HOH | 603 | −2.982 | 21.809 | 7.218 | 1.00 | 69.39 |
| ATOM | 1627 | O1 | HOH | 604 | 13.293 | 8.016 | 2.296 | 1.00 | 61.13 |
| ATOM | 1628 | O1 | HOH | 605 | 3.299 | 11.650 | 51.045 | 1.00 | 78.04 |
| ATOM | 1629 | O1 | HOH | 606 | −16.971 | 17.233 | 50.226 | 1.00 | 68.89 |
| ATOM | 1630 | O1 | HOH | 607 | 2.846 | 12.738 | 39.593 | 1.00 | 71.60 |
| ATOM | 1631 | O1 | HOH | 608 | −3.147 | −2.544 | 9.531 | 1.00 | 58.55 |
| ATOM | 1632 | O1 | HOH | 609 | −4.861 | 3.305 | 80.691 | 1.00 | 60.83 |
| ATOM | 1633 | O1 | HOH | 610 | 6.834 | 13.354 | 71.357 | 1.00 | 64.45 |
| ATOM | 1634 | O1 | HOH | 611 | 2.963 | 4.487 | 27.971 | 1.00 | 48.70 |
| ATOM | 1635 | O1 | HOH | 612 | −2.162 | 14.623 | 70.194 | 1.00 | 72.85 |
| ATOM | 1636 | O1 | HOH | 613 | −8.983 | 11.246 | 91.235 | 1.00 | 68.16 |
| ATOM | 1637 | O1 | HOH | 614 | −1.414 | 15.727 | 89.255 | 1.00 | 63.90 |
| ATOM | 1638 | O1 | HOH | 615 | 1.353 | 25.378 | 70.806 | 1.00 | 68.29 |
| ATOM | 1639 | O1 | HOH | 616 | 0.315 | 14.016 | 78.504 | 1.00 | 59.91 |
| ATOM | 1640 | O1 | HOH | 617 | 4.477 | 9.852 | 70.092 | 1.00 | 79.40 |
| ATOM | 1641 | O1 | HOH | 618 | −12.259 | 11.420 | 46.776 | 1.00 | 58.20 |
| ATOM | 1642 | O1 | HOH | 619 | 2.679 | 4.422 | 32.337 | 1.00 | 49.91 |
| ATOM | 1643 | O1 | HOH | 620 | −17.285 | 12.212 | 71.786 | 1.00 | 68.80 |
| ATOM | 1644 | O1 | HOH | 621 | −17.043 | 8.933 | 85.930 | 1.00 | 71.94 |
| ATOM | 1645 | O1 | HOH | 622 | −12.833 | 21.451 | 57.231 | 1.00 | 69.93 |
| ATOM | 1646 | O1 | HOH | 623 | −16.934 | 18.199 | 72.267 | 1.00 | 64.58 |
| ATOM | 1647 | O1 | HOH | 624 | −1.857 | 26.990 | 67.917 | 1.00 | 52.07 |
| ATOM | 1648 | O1 | HOH | 625 | −13.989 | 23.289 | 43.768 | 1.00 | 68.81 |
| ATOM | 1649 | O1 | HOH | 626 | −8.279 | 10.487 | 46.008 | 1.00 | 64.05 |
| ATOM | 1650 | O1 | HOH | 627 | −8.410 | 14.523 | 46.407 | 1.00 | 94.70 |
| ATOM | 1651 | O1 | HOH | 628 | −14.238 | 29.716 | 34.051 | 1.00 | 74.01 |
| ATOM | 1652 | O1 | HOH | 629 | 2.497 | 10.302 | 52.957 | 1.00 | 78.36 |
| ATOM | 1653 | O1 | HOH | 630 | 12.781 | 18.085 | 14.371 | 1.00 | 59.38 |
| ATOM | 1654 | O1 | HOH | 631 | −13.379 | 8.527 | 74.408 | 1.00 | 72.72 |
| ATOM | 1655 | O1 | HOH | 632 | −8.436 | 24.292 | 72.901 | 1.00 | 80.80 |
| ATOM | 1656 | O1 | HOH | 633 | −11.927 | 12.370 | 43.399 | 1.00 | 72.78 |
| ATOM | 1657 | O1 | HOH | 634 | 3.105 | 21.424 | 61.107 | 1.00 | 80.29 |
| ATOM | 1658 | O1 | HOH | 635 | 1.552 | 18.331 | 85.585 | 1.00 | 75.99 |
| ATOM | 1659 | O1 | HOH | 636 | −13.619 | 20.355 | 61.490 | 1.00 | 63.28 |
| ATOM | 1660 | O1 | HOH | 637 | −2.200 | 8.533 | 10.367 | 1.00 | 63.23 |
| ATOM | 1661 | O1 | HOH | 638 | 3.589 | 19.637 | 44.461 | 1.00 | 47.03 |
| ATOM | 1662 | O1 | HOH | 639 | −1.573 | 21.818 | 9.438 | 1.00 | 68.93 |
| ATOM | 1663 | O1 | HOH | 640 | 6.258 | 22.557 | 73.322 | 1.00 | 58.13 |
| ATOM | 1664 | O1 | HOH | 641 | −13.066 | 10.161 | 44.534 | 1.00 | 63.83 |
| ATOM | 1665 | O1 | HOH | 642 | −9.747 | 28.802 | 92.952 | 1.00 | 80.11 |
| ATOM | 1666 | O1 | HOH | 643 | −5.127 | 4.127 | 4.706 | 1.00 | 79.47 |
| ATOM | 1667 | O1 | HOH | 644 | −13.918 | 12.183 | 81.003 | 1.00 | 58.64 |
| ATOM | 1668 | O1 | HOH | 645 | 19.938 | 9.499 | 13.944 | 1.00 | 87.18 |
| ATOM | 1669 | HG | MER | 701 | 9.336 | 0.982 | 15.636 | 0.20 | 58.21 |
| ATOM | 1670 | HG | MER | 702 | 9.841 | 5.199 | 14.508 | 0.20 | 67.41 |
| ATOM | 1671 | HG | MER | 703 | −7.127 | 10.772 | 57.593 | 0.20 | 79.35 |
| ATOM | 1672 | C1 | DMA | 801 | −13.170 | 18.614 | 25.453 | 1.00 | 77.87 |
| ATOM | 1674 | C1′ | DMA | 801 | −11.027 | 17.896 | 26.233 | 1.00 | 77.70 |
| ATOM | 1675 | C2′ | DMA | 801 | −9.669 | 18.447 | 26.518 | 1.00 | 75.99 |
| ATOM | 1676 | C3′ | DMA | 801 | −8.688 | 17.714 | 25.612 | 1.00 | 72.89 |
| ATOM | 1677 | C4′ | DMA | 801 | −7.247 | 18.219 | 25.835 | 1.00 | 69.58 |
| ATOM | 1678 | C5′ | DMA | 801 | −6.263 | 17.478 | 24.914 | 1.00 | 65.32 |
| ATOM | 1679 | C6′ | DMA | 801 | −6.218 | 16.101 | 25.451 | 1.00 | 61.15 |
| ATOM | 1680 | C7′ | DMA | 801 | −5.365 | 15.188 | 24.661 | 1.00 | 59.83 |
| ATOM | 1681 | C8′ | DMA | 801 | −5.575 | 13.886 | 25.325 | 1.00 | 64.73 |
| ATOM | 1682 | C9′ | DMA | 801 | −4.915 | 12.693 | 24.717 | 1.00 | 64.11 |
| ATOM | 1683 | C10 | DMA | 801 | −5.345 | 11.526 | 25.553 | 1.00 | 67.12 |
| ATOM | 1684 | O1 | DMA | 801 | −11.982 | 18.934 | 26.182 | 1.00 | 79.61 |
| ATOM | 1685 | O5 | DMA | 801 | −12.859 | 18.362 | 24.066 | 1.00 | 75.75 |

The following abbreviations are used in Table 2:

“Atom Type” refers to the element whose coordinates are provided. The first letter in the column defines the element. For example, that beginning with “O” indicates oxygen, “C” carbon, “N” nitrogen, and “S” sulfur. But “HG” indicates mercury.

“A.A.” refers to amino acid. In this column, HOH indicates water molecule, MER does mercury atom, and DMA does the atom of detergent.

In the column of “A.A. No.”, 1-185 indicate 185 of amino acids in RRF, 302-645 do water molecules, 701-703 do mercury atoms, and 801 does the detergent.

“X, Y and Z” provide the Cartesian coordinates of the element.

“B” is a thermal factor that measures movement of the atom around its atom center.

“OCC” refers to occupancy and represents the degree that the atom occupies the particular coordinate. For example, 1.0 indicates that one atom is located on the coordinate. 0.2 indicates that 0.2 of atom is located.

While the present invention has been described with respect to a certain preferred embodiment only, other modifications and variations may be made without departing from the spirit and scope of the present invention as set forth in the following claims.

### REFERENCES

Agrawal, R. K., Penczek, P., Grassucci, R. A., Li, Y., Leith, A., Nierhaus, K. H., and Frank, J. (1996). Direct visualization of A-, P-, and E-site transfer RNAs in the Escherichia coli ribosome. Science 271, 1000-1002.

Agrawal, R. K., Penczek, P., Grassucci, R. A., and Frank, J. (1998). Visualization of elongation factor G on the Escherichia coli 70S ribosome: the mechanism of translocation. Proc Natl Acad Sci USA 95, 6134-6138.

Agrawal, R. K., Heagle, A. B., Penczek, P., Grassucci, R. A., and Frank, J. (1999). EF-G dependent GTP hydrolysis induces translocation accompanied by large changes in the 70S ribosome. Nat. Struct. Biol. 6, 643-647.

Ban, N., Nissen, P., Hansen, J., Capel, M., Moore, P. B., and Steitz, T. A. (1999). Placement of protein and RNA structures into a 5 resolution map of the 50S ribosomal subunit. Nature 400, 841-847.

Blundell et al., 1976, Protein Crystallography, Academic Press.

Brock, S., Szkaradkiewicz, K., and Sprinzl, M. (1998). Initiation factors of protein biosynthesis in bacteria and their structural relationship to elongation and termination factors. Mol Microbiol. 29, 409-417.

Brunger, A. T., Adams, P. D., Clore, G. M., DeLano, W. L., Gros, P., Grosse-Kunstleve, R. W., Jiang, J. S., Kuszewski, J., Nilges, M., Pannu, N. S., Read, R. J., Rice, L. M., Simonson, T., and Warren, G. L. (1998). Crystallography & NMR system: A new software suite for macromolecular structure determination. Acta Crystallogr. D 54, 905-921.

Bullough, P. A., Hughson, F. M., Skehel, J. J., and Wiley, D.C. (1994). Structure of influenza haemagglutinin at the pH of membrane fusion. Nature 371, 37-43.

Collaborative Computational Project Number 4 (1994). The CCP4 suite: programs for protein crystallography. Acta Crystallogr. D 50, 760-763.

Colovos, C., and Yeates, T. O. (1993). Verification of protein structures: patterns of nonbonded atomic interactions. Protein Sci. 2, 1511-1519.

Fortelle, E. d. L., and Bricogne, G. (1997). Maximum likelihood heavy atom refinement for multiple isomorphous replacement and multianomalous diffraction methods. Methods Enzymol. 276, 472-494.

Freistroffer, D. V., Pavlov, M. Y., MacDougall, J., Buckingham, R. H., and Ehrenberg, M. (1997). Release factor RF3 in E.coli accelerates the dissociation of release factors RF1 and RF2 from the ribosome in a GTP-dependent manner. EMBO J. 16, 4126-4133.

Grentzmann, G., Kelly, P. J., Laalami, S., Shuda, M., Firpo, M. A., Cenatiempo, Y., and Kaji, A. (1998). Release factor RF-3 GTPase activity acts in disassembly of the ribosome termination complex. RNA 4, 973-983.

Heurgue-Hamard, V., Karimi, R., Mora, L., MacDougall, J., Lebocuf, C., Grentzmann, G., Ehrenberg, M., and Buckingham, R. H. (1998). Ribosome release factor RF4 and termination factor RF3 are involved in dissociation of peptidyl-tRNA from the ribosome. EMBO J. 17, 808-816.

Janosi, L., Shimizu, I., and Kaji, A. (1994). Ribosome recycling factor (ribosome releasing factor) is essential for bacterial growth. Proc Natl Acad Sci USA 91, 4249-4253.

Janosi, L., Ricker, R., and Kaji, A. (1996). Dual functions of ribosome recycling factor in protein biosynthesis: disassembling the termination complex and preventing translational errors. Biochimie 78, 959-969.

Janosi, L., Mottagui-Tabar, S., Isaksson, L. A., Sekine, Y., Ohtsubo, E., Zhang, S., Goon, S., Nelken, S., Shuda, M., and Kaji, A. (1998). Evidence for in vivo ribosome recycling, the fourth step in protein biosynthesis. EMBO J. 17, 1141-1151.

Jones, T. A., Zou, J.-Y., Cowan, S. W., and Kjeldgaard, M. (1991). Improved methods for binding protein models in electron density maps and the location of errors in these models. Acta Crystallogr. A 47, 110-119.

Kaji, A., Teyssier, E., and Hirokawa, G. (1998). Disassembly of the post-termination complex and reduction of translational error by ribosome recycling factor (RRF)-A possible new target for antibacterial agents. Biochem Biophys Res Commun. 250, 1-4.

Kanai, T., Takeshita, S., Atomi, H., Umemura, K., Ueda, M., and Tanaka, A. (1998). A regulatory factor, Fil1p, involved in derepression of the isocitrate lyase gene in Saccharomyces cerevisiae—a possible mitochondrial protein necessary for protein synthesis in mitochondria. Eur J. Biochem. 256, 212-220.

Karimi, R., Pavlov, M. Y., Buckingham, R. H., and Ehrenberg, M. (1999). Novel roles for classical factors at the interface between translation termination and initiation. Mol Cell 3, 601-609.

Kissinger, C. R., and Gehlhaar, D. K. (1998). EPMR, A program for crystallographic molecular replacement by evolutionary search, versions 2.1 (Agouron Pharmaceticals, Inc.).

Kraulis, P. J. (1991). MOLSCRIPT: a program to produce both detailed and schematic plots of protein. J. Appl. Crystallogr. 24, 946-950.

Laskowski, R. A., Macarthur, M. W., Moss, D. S., and Thornton, J. M. (1993). PROCHECK: a program to check the stereochemical quality of protein structures. J. Appl. Crystallogr. 26, 283-291.

Liljas, A., and Garber, M. (1995). Ribosomal proteins and elongation factor. Current Opinion in Str. Biol. 5, 721-727.

Lupas, A. (1996). Coiled coils: new structures and new functions. Trends Biochem. Sci. 21, 375-382.

Martemyanov, K. A., and Gudkov, A. T. (1999). Domain IV of elongation factor G from Thermus thermophilus is strictly required for translocation. FEBS Lett 452, 155-159.

Nakamura, Y., Ito, K., and Isaksson, L. A. (1996). Emerging understanding of translation termination. Cell 87, 147-150.

Nevskaya, N., Tishchenko, S., Nikulin, A., al-Karadaghi, S., Liljas, A., Ehresmann, B., Ehresmann, C., Garber, M., and Nikonov, S. (1998). Crystal structure of ribosomal protein S8 from Thermus thermophilus reveals a high degree of structural conservation of a specific RNA binding site. J. Mol. Biol. 279, 233-244.

Kim, K. K., Min, K., and Suh, S. W. (2000). Crystal structure of the ribosome recycling factor from Escherichia coli. EMBO J. 19(10), 2363-2370.

Nicholls, A., Sharp, K. A., and Honig, B. (1991). Protein folding and association: insights from the interfacial and thermodynamic properties of hydrocarbons. Prot. Struct. Funct. Genet. 11, 281-296.

Nissen, P., Kjeldgaard, M., Thirup, S., Polekhina, G., Reshetnikova, L., Clark, B. F., and Nyborg, J. (1995). Crystal structure of the ternary complex of Phe-tRNAPhe, EF-Tu, and a GTP analog. Science 270, 1464-1472.

Otwinowski, Z., and Minor, W. (1997). Processing of X-ray diffraction data collected in oscillation model. Methods Enzymol. 276, 307-326.

Pavlov, M. Y., Freistroffer, D. V., MacDougall, J., Buckingham, R. H., and Ehrenberg, M. (1997a). Fast recycling of Escherichia coli ribosomes requires both ribosome recycling factor (RRF) and release factor RF3. EMBO J. 16, 4134-4141.

Pavlov, M. Y., Freistroffer, D. V., Heurgue-Hamard, V., Buckingham, R. H., and Ehrenberg, M. (1997b). Release factor RF3 abolishes competition between release factor RF1 and ribosome recycling factor (RRF) for a ribosome binding site. J. Mol. Biol. 273, 389-401.

Rolland, N., Janosi, L., Block, M. A., Shuda, M., Teyssier, E., Miege, C., Chenicnlet, C., Carde, J. P., Kaji, A., and Joyard, J. (1999). Plant ribosome recycling factor homologue is a chloroplastic protein and is bactericidal in Escherichia coli carrying temperature-sensitive ribosome recycling factor. Proc Natl Acad Sci USA 96, 5464-5469.

Ryoji, M., Berland, R., and Kaji, A.(1981). Reinitiation of translation from the triplet next to the amber termination codon in the absence of ribosome-releasing factor. Proc Natl Acad Sci USA 78, 5973-5977.

Wang, R., Liu, L., and Lai, L. (1998). SCORE: A New Empirical Method for Estimating the Bindind Affinity of a Protein-Ligand Complex. J. Mol. Model 4, 379-394.

Yun, J., Kim, W., Ha, S. S., Eom, S.-H., Suh, S. W., and Kim, K. K. (2000). Crystallization and preliminary crystallographic studies of ribosome recycling factor from Excherichia coli. Acta Crystallogr D 56, 84-85.

What is claimed is:

1. A method of developing an inhibitor of a ribosome recycling factor (RRF), comprising:
providing data of a three-dimensional structure of RRF, the three-dimensional structure comprising a coiled-coil domain;
identifying one or more amino acids of the coiled-coil domain that play a critical role in binding with ribosome A-site;
obtaining a local three-dimensional structure in the vicinity of the one or more amino acids from the data of the three-dimensional structure of RRF; and
designing or searching for substances comprising a three-dimensional structure, which is either substantially complimentary or substantially similar to the local three-dimensional structure in the vicinity of the one or more amino acids.

2. The method of claim 1, wherein the three-dimensional structure of RRF comprises a hydrophobic cleft, in which a compound is inserted.

3. The method of claim 2, wherein at least one amino acids participates in hydrophobic binding with the compound, and wherein the at least one participating amino acids are selected from the group consisting of Thr-106, Arg-31, Pro-103, Leu-36, Leu-37, Ile-40, Leu-87, Leu-89 and Leu-102.

4. The method of claim 2, wherein the compound comprises 3-12 carbon straight-chain alkyl group.

5. The method of claim 4, wherein the compound further comprises a non-hydrophobic substituent group.

6. The method of claim 2, wherein the compound is decyl-β-D-maltopyranoside.

7. The method of claim 1, wherein the coiled-coil domain comprises three helices represented by SEQ ID NOS. 5-7 for Escherichia coli, SEQ ID NOS. 8-10 for Haemophilus influenzae, SEQ ID NOS. 11-13 for Psuedomonas aeuruginosa, and SEQ ID NOS. 14-16 for Bacillus subtillis.

8. The method of claim 1, wherein the three-dimensional structure of RRF is obtained by X-ray crystallography of RRF.

9. The method of claim 8, wherein the provision of the three-dimensional structure of RRF comprises:
providing an RRF;
crystallizing the RRF in the presence of a detergent comprising a hydrophobic portion; and
conducting X-ray diffraction analysis of the crystallized RRF combined with the detergent.

10. The method of claim 9, wherein the hydrophobic portion of the detergent is inserted into a cleft of the RRF.

11. The method of claim 1, wherein the one or more amino acids of the coiled-coil domain that play a critical role are selected from the group consisting of amino acid Nos. 1, 3, 4, 6, 7, 8, 10, 11, 12, 14, 15, 17, 18, 19, 107, 108, 109, 111, 112, 114, 115, 116, 118, 119, 120, 122, 123, 125, 126, 127, 129, 130, 132, 133, 134, 136, 137, 138, 140, 141, 143, 144, 145, 146, 147, 148, 150, 151, 152, 153, 155, 156, 158, 159, 161, 162, 164, 165, 166, 167, 169, 170, 172, 173, 174, 176, 177, 179, 180 and 181 in SEQ ID NOS 1-4.

12. The method of claim 1, wherein the identifying comprises:
providing a microorganism comprising RRF;
mutating the RRF of the microorganism by replacing one or more amino acids of the coiled-coil domain;
observing growth of the microorganism with the mutated RRF; and
determining whether the one or more amino acids replaced play a critical role in binding with a ribosome A-site from the observation of the growth.

13. The method of claim 1, wherein the designing or searching comprises conducting computerized modeling with the data of the three-dimensional structure of RRF.

14. The method of claim 1, further comprising selecting a substance comprising a three-dimensional structure substantially complimentary or substantially similar to the local three-dimensional structure.

15. The method of claim 14, wherein the designing or searching further comprises modifying the selected substance.

16. The method of claim 14, further comprising testing RRF inhibition activity of the selected substance.

17. The method of claim 16, wherein the testing is conducted in vitro or in vivo.

18. The method of claim 14, further comprising synthesizing the selected substance.

19. The method of claim 18, wherein the synthesis is biologically carried out.

20. The method of claim 18, wherein the synthesis is chemically carried out.

21. The method of claim 1, wherein the substance is selected from the group consisting of amino acids, peptides, polypeptides and compounds comprising peptides.


SEQUENCE LISTING (16 sequences)

SEQ ID NO: 1 - protein, 185 aa, E. coli [sequence omitted]

SEQ ID NO: 2 - protein, 185 aa, Haemophilus influenzae
MLNQIKKDAQDRMEKSLEALKGHISKIRTGRAQPSLLDAIQVEYYGAATPLRQLANVVAEDARTLAVTVFDRSLISAVEKAILTSDLGLNPSSAGTTIRVPLPPLTEEERRDLIKIVKGEGEQGKVAVRNVRRDANDKIKALLKDKEISENEQHKAEEEIQKITDIYIKKVDEVLADKEKELMDF

SEQ ID NO: 3 - protein, 185 aa, Pseudomonas aeruginosa
MINEIKKEAQERMGKTLEALGHAFAKIRTGRAHPSILDSVMVSYYGADTPLRQVANVTVEDSRTLALAVFDKSMIQAVEKAIMTSDLGLNPATAGTTIRVPMPALTEETRKGYTKQARAEAEQARVSVRNIRRDALAQLKDLQKEKEISEDEERRAGDDVQKLTDKFIGEIEKALEAKEADLMAV

SEQ ID NO: 4 - protein, 185 aa, Bacillus subtilis
MSKEVLTQTKEKMEKAIAAYQRELATVRAGRANPSLLDKVTVEYYGAQTPLNQLSSINVPEARMLVITPYDKTAIGDIEKAILKADLGLTPTSDGNMIRIAIPALTEERRKELVKVVKKYAEEAKVAVRNVRRDANDDLKKLEKNGDITEDELRASTEDVQKLTDEYVSKIDSVTKDKEKEIMEV

SEQ ID NO: 5 - protein, 25 aa, E. coli
MISDIRKDAEVRMDKCVEAFKTQIS

SEQ ID NO: 6 - protein, 40 aa, E. coli
TEERRKDLTKIVRGEAEQARVAVRNVRRDANDKVKALLKD

SEQ ID NO: 7 - protein, 37 aa, E. coli
SEDDDRRSQDDVQKLTDAAIKKIEAALADKEAELMQF

SEQ ID NO: 8 - protein, 25 aa, Haemophilus influenzae
MLNQIKKDAQDRMEKSLEALKGHIS

SEQ ID NO: 9 - protein, 40 aa, Haemophilus influenzae
TEERRRDLIKIVKGEGEQGKVAVRNVRRDANDKIKALLKD

SEQ ID NO: 10 - protein, 37 aa, Haemophilus influenzae
SENEQHKAEEEIQKITDIYIKKVDEVLADKEKELMDF

SEQ ID NO: 11 - protein, 25 aa, Pseudomonas aeruginosa
MINEIKKEAQERMGKTLEALGHAFA

SEQ ID NO: 12 - protein, 40 aa, Pseudomonas aeruginosa
TEETRKGYTKQARAEAEQARVSVRNIRRDALAQLKDLQKE

SEQ ID NO: 13 - protein, 37 aa, Pseudomonas aeruginosa
SEDEERRAGDDVQKLTDKFIGEIEKALEAKEADLMAV

SEQ ID NO: 14 - protein, 25 aa, Bacillus subtilis
MSKEVLTQTKEKMEKAIAAYQRELA

SEQ ID NO: 15 - protein, 40 aa, Bacillus subtilis
TEERRKELVKVVKKYAEEAKVAVRNVRRDANDDLKKLEKN

SEQ ID NO: 16 - protein, 37 aa, Bacillus subtilis
TEDELRASTEDVQKLTDEYVSKIDSVTKDKEKEIMEV